(19) United States
(12) Patent Application Publication
(10) Pub. No.: US 2025/0059594 A1
(43) Pub. Date: Feb. 20, 2025

(54) Compositions and Methods for Detecting Toxigenic Clostridium Difficile

(71) Applicant: Gen-Probe Incorporated, San Diego, CA (US)

(72) Inventor: Patrick Lynn Peterson, San Marcos, CA (US)

(73) Assignee: Gen-Probe Incorporated, San Diego, CA (US)

(21) Appl. No.: 18/825,695

(22) Filed: Sep. 5, 2024

Related U.S. Application Data
(62) Division of application No. 16/769,229, filed on Jun. 2, 2020, now Pat. No. 12,110,540.
(60) Provisional application No. 62/599,494, filed on Dec. 15, 2017.

(51) Int. Cl. C12Q 1/6827; C12Q 1/689

(52) U.S. Cl. CPC C12Q 1/6827; C12Q 1/689; C12Q 2537/143; C12Q 2561/109; C12Q 2565/101; C12Q 2600/156; C12Q 2600/166

(57) ABSTRACT

Provided herein are compositions, kits, and methods for detecting at least one of a C. difficile tcdA, tcdB, tcdC, cdtA, or cdtB nucleic acid in a sample. In some embodiments, one or more alleles of tcdC such as 117del tcdC or 184T tcdC are detected.

### CROSS-REFERENCE TO RELATED APPLICATIONS

This application is a divisional application of U.S. application Ser. No. 16/769,229, having a 35 U.S.C. § 371(c) date of Jun. 2, 2020, and which is a § 371 Application of PCT/US2018/065470, filed on Dec. 13, 2018, which claims the benefit under 35 U.S.C. § 119(e) of U.S. Provisional Application No. 62/599,494, filed Dec. 15, 2017, the contents of each of which are hereby incorporated by reference herein.

### SEQUENCE LISTING

The present application is filed with a Sequence Listing in electronic XML format. The Sequence Listing is provided as a file entitled “01159-0015-01US-Revised.xml” created on Nov. 2, 2024, and which is 628,343 bytes in size. The information in the electronic XML format of the sequence listing is incorporated herein by reference in its entirety.

### INTRODUCTION AND SUMMARY

The embodiments herein are directed to the field of detecting infectious agents, more specifically by using compositions and methods to detect toxigenic Clostridium difficile nucleic acids. Clostridium difficile as used herein is synonymous with Clostridioides difficile.

C. difficile is a gram-positive bacterium responsible for gastrointestinal infections, diarrhea including antibiotic-associated diarrhea, and pseudomembranous colitis. C. difficile can also colonize individuals asymptomatically. It can undergo sporulation to form spores that are resistant to many conditions that would kill vegetative bacteria, including heat, antibiotic exposure, and more. As such, spores in a patient's gastrointestinal tract (which may or may not have been associated with symptomatic infection as opposed to asymptomatic colonization) can survive antibiotic treatment and then germinate to cause a subsequent illness. The difficulty of inactivating C. difficile spores, which are excreted in feces and also naturally occurring in soil, means that some decontamination procedures such as antiseptic solutions and ultraviolet treatment may not be effective, and thus contamination following fecal or soil exposure can persist and serve to spread infections.

C. difficile strains can produce various toxins that contribute to diarrhea and other symptoms. These include toxin A and toxin B, encoded by the tcdA and tcdB genes, respectively, and found in the Pathogenic Locus, and the binary toxin, which has two subunits encoded by the cdtA and cdtB genes in the Cdt locus. Not all toxin genes occur in all strains. Non-toxinogenic strains do not express the toxins and have been suggested based on animal data to confer a protective effect, perhaps by competing with pathogenic strains for colonization in the gastrointestinal tract. Toxins A and B were characterized earlier than the binary toxin and were historically considered necessary for pathogenicity, although pathogenicity has recently been reported in a strain designated RA09-70, which is negative for Toxin B. See Monot et al., Sci Rep. 2015; 5: 15023, available in PubMed Central as PMCID: PMC4597214.

Furthermore, certain mutations in the tcdC gene, which encodes a regulator of tcdA and tcdB expression, can result in increased levels of toxins A and B and a hypervirulent phenotype. Some relatively recent reports have indicated that the binary toxin can in some cases suffice to cause colitis in the absence of toxins A and B. Presence of both the Cdt locus and the Pathogenic Locus has also been linked to more serious disease.

Rapid and reliable identification of toxigenic C. difficile, including hypervirulent C. difficile in particular, is important for facilitating appropriate responses to both treat infected subjects and contain and limit spread of the bacterial infection, including antibiotic therapy and other infection control measures such as rigorous disinfection and hygienic precautions. Thus, compositions and methods for rapid and reliable nucleic acid-based detection of toxigenic C. difficile are desirable.

Nucleic acid-based detection of C. difficile is, however, complicated by several factors. As noted above, non-toxinogenic strains are not thought to cause disease and may be protective, so nucleic-acid based approaches logically focus on the toxin-coding genes and tcdC, the genotype of which can contribute to hypervirulence. Because other Clostridium species (e.g., C. soldelli) can carry related toxin genes, cross-reactivity with sequences from other Clostridium species can be an issue. Furthermore, a number of different alleles of the genes present in the PaLoc and CdtLoc exist in different C. difficile strains, which can further complicate their detection. For example, sequence variants of tcdB can result in false negatives if they fail to hybridize sufficiently with amplification or detection oligomers. As a further example, at least four tcdC variants at position 117 have been described, only one of which results in hypervirulence. Efforts have been made to specifically detect alleles of tcdC associated with hypervirulence, but these may be subject to erroneous results. For example, 117T tcdC alleles do not promote hypervirulence but in some assays may give rise to a false positive signal indicating hypervirulence. Additionally, some assays do not detect the two tcdC alleles that are associated with hypervirulence, 117del and 184T, making the assays prone to false negative results.

Accordingly, there is a need for compositions and methods that can provide rapid and sensitive amplification of various alleles of the C. difficile toxin and toxin-related target sequences, which can be used for accurate identification of toxigenic C. difficile despite its intraspecific heterogeneity, and which can discriminate alleles that are associated with hypervirulence from those that are not, thereby assisting in the identification of hypervirulent and non-hypervirulent strains. In various embodiments, this disclosure aims to meet these needs, provide other benefits, or at least provide the public with a useful choice.

Provided herein are the following embodiments. Embodiment 1 is a composition or kit comprising forward and reverse tcdC amplification oligomers and further comprising one or both of a first and a second tcdC detection oligomer, wherein: the first tcdC detection oligomer, if present, is configured to specifically hybridize to a first tcdC sequence at a site comprising position 117 thereof, but exhibits distinguishably different hybridization to a second tcdC sequence, wherein the first tcdC sequence is the sequence of SEQ ID NO: 3 and the second tcdC sequence is the sequence of SEQ ID NO: 4; the second tcdC detection oligomer, if present, is configured to specifically hybridize to the sequence of a third tcdC sequence at a site comprising position 184 thereof, but exhibits distinguishably different hybridization to the sequence of a fourth tcdC sequence, wherein the third tcdC sequence is the sequence of SEQ ID NO: 5 and the fourth tcdC sequence is the sequence of SEQ ID NO: 2; and the forward and reverse tcdC amplification oligomers are configured to produce a tcdC amplicon, wherein the tcdC amplicon comprises position 117 of SEQ ID NO: 3 or SEQ ID NO: 4 if the first tcdC detection oligomer is present and/or position 184 of SEQ ID NO: 5 if the second tcdC detection oligomer is present.

Embodiment 2 is a composition comprising forward and reverse tcdC amplification oligomers, one or both of a first tcdC detection oligomer and a second tcdC detection oligomer, forward and reverse tcdA amplification oligomers, and at least one tcdA detection oligomer, wherein: the first tcdC detection oligomer, if present, is configured to specifically hybridize to a first tcdC sequence, which is the sequence of SEQ ID NO: 3, at a site comprising position 117 thereof; the second tcdC detection oligomer, if present, is configured to specifically hybridize to a third tcdC sequence, which is the sequence of SEQ ID NO: 5, at a site comprising position 184 thereof; the forward and reverse tcdC amplification oligomers are configured to produce a tcdC amplicon, wherein the tcdC amplicon comprises position 117 of SEQ ID NO: 3 if the first tcdC detection oligomer is present and/or position 184 of SEQ ID NO: 5 if the second tcdC detection oligomer is present; the forward and reverse tcdA amplification oligomers are configured to produce a tcdA amplicon having a size of from 80 to 400 nucleotides; and the tcdA detection oligomer is configured to specifically hybridize to the tcdA amplicon.

Embodiment 3 is a method of detecting a C. difficile tcdC allele, the C. difficile tcdC allele comprising a 117del mutation or a 184T mutation, the method comprising: preparing a composition according to embodiment 1 or 2 which further comprises a sample comprising or suspected of comprising C. difficile nucleic acid; subjecting the composition to amplification conditions; and detecting the presence of the C. difficile 117del tcdC allele or 184T tcdC allele in the sample by determining whether at least one of the first tcdC detection oligomer or the second tcdC detection oligomer hybridized to a tcdC amplicon.

Embodiment 4 is a composition or kit comprising either or both of: (i) a first tcdC detection oligomer and a first tcdC primary probe oligomer; and (ii) a second tcdC detection oligomer and a second tcdC primary probe oligomer, wherein: the at least one first tcdC detection oligomer, if present, is an invader oligomer configured to specifically hybridize to a first tcdC sequence at a site comprising position 117 thereof, but exhibits distinguishably different hybridization to a second tcdC sequence, wherein the first tcdC sequence is the sequence of SEQ ID NO: 3 and the second tcdC sequence is the sequence of SEQ ID NO: 4; the first tcdC primary probe oligomer, if present, is configured to form an invasive cleavage substrate with the first tcdC detection oligomer in the presence of the first tcdC sequence; the second tcdC detection oligomer, if present, is an invader oligomer configured to specifically hybridize to the sequence of a third tcdC sequence at a site comprising position 184 thereof, but exhibits distinguishably different hybridization to the sequence of a fourth tcdC sequence, wherein the third tcdC sequence is the sequence of SEQ ID NO: 5 and the fourth tcdC sequence is the sequence of SEQ ID NO: 2; and the second tcdC primary probe oligomer, if present, is configured to form an invasive cleavage substrate with the second tcdC detection oligomer in the presence of the third tcdC sequence.

Embodiment 5 is a method of detecting a C. difficile tcdC allele, the C. difficile tcdC allele comprising a 117del mutation or a 184T mutation, the method comprising: preparing a composition according to embodiment 4 which further comprises a sample comprising or suspected of comprising C. difficile nucleic acid; subjecting the composition to invasive cleavage reaction conditions; and detecting the presence of the mutant C. difficile tcdC sequence in the sample by determining whether at least one of the first tcdC primary probe or the second tcdC primary probe underwent cleavage.

Embodiment 6 is the method of any one of embodiments 3 or 5, wherein the C. difficile tcdC allele comprises at least one of a 117del mutation and a 184T mutation.

Embodiment 7 is the method of any one of embodiments 3, 5, or 6, wherein the method is configured to generate a positive signal in the presence of 117del mutant tcdC.

Embodiment 8 is the method of any one of embodiments 3 or 5-7, wherein the method is configured not to generate a positive signal in the presence of 117T tcdC.

Embodiment 9 is the method of any one of embodiments 3 or 5-8, wherein the method is configured not to generate a positive signal in the presence of 117D tcdC.

Embodiment 10 is the composition, kit, or method of any one of the preceding embodiments, wherein the first tcdC detection oligomer is present.

Embodiment 11 is the composition, kit, or method of embodiment 10, wherein the first tcdC detection oligomer competes for hybridization to a tcdC nucleic acid under stringent conditions with an oligomer having a sequence consisting of SEQ ID NO: 86-92, 183, 192, or 193.

Embodiment 12 is the composition, kit, or method of embodiment 10 or 11, wherein the first tcdC detection oligomer comprises the sequence of any one of SEQ ID NOs: 87-92, with up to two mismatches.

Embodiment 13 is the composition, kit, or method of embodiment 12, wherein the first tcdC detection oligomer comprises the sequence of SEQ ID NO: 88, with up to two mismatches.

Embodiment 14 is the composition, kit, or method of embodiment 10 or 11, wherein the first tcdC detection oligomer comprises the sequence of SEQ ID NO: 192 or 193, with up to two mismatches.

Embodiment 15 is the composition, kit, or method of embodiment 10 or 11, wherein the first tcdC detection oligomer comprises the sequence of SEQ ID NO: 86 with up to two mismatches.

Embodiment 16 is the composition, kit, or method of embodiment 10 or 11, wherein the first tcdC detection oligomer comprises the sequence of SEQ ID NO: 183 with up to two mismatches.

Embodiment 17 is the composition, kit, or method of any one embodiments 10-16, wherein the first tcdC detection oligomer has no mismatches to the 1, 2, 3, 4, 5, 6, 7, 8, 9, or 10 3′-terminal nucleotides of SEQ ID NO: 86-92, 183, 192, or 193.

Embodiment 18 is the composition, kit, or method of embodiment 17, wherein the first tcdC detection oligomer comprises the sequence of of SEQ ID NO: 86 or 183.

Embodiment 19 is the composition, kit, or method of embodiment 11, wherein the first tcdC detection oligomer comprises the sequence of any one of SEQ ID NOs: 87-92, 192, or 193.

Embodiment 20 is the composition, kit, or method of embodiment 19, wherein: (i) the first tcdC detection oligomer comprises the sequence any one of SEQ ID NOs: 89-92; and (ii) the composition or kit further comprises an additional first tcdC detection oligomer that comprises the sequence any one of SEQ ID NOs: 89-92, which is different from the first tcdC detection oligomer.

Embodiment 21 is the composition, kit, or method of any one of the preceding embodiments, wherein the composition or kit further comprises at least one first tcdC primary probe oligomer that is configured to form an invasive cleavage structure in the presence of the first tcdC detection oligomer and a polynucleotide comprising the sequence of SEQ ID NO: 3.

Embodiment 22 is the composition, kit, or method of embodiment 21, wherein the first tcdC primary probe oligomer comprises the sequence of any one of SEQ ID NOs: 93-137 or 184-187, with up to two mismatches.

Embodiment 23 is the composition, kit, or method of embodiment 21 or 22, wherein the first tcdC primary probe oligomer competes for hybridization to a tcdC nucleic acid under stringent conditions with an oligomer having a sequence consisting of SEQ ID NO: 138-182 or 188-191.

Embodiment 24 is the composition, kit, or method of any one of embodiments 21-23, wherein the first tcdC primary probe oligomer comprises the sequence of SEQ ID NO: 115, with up to two mismatches.

Embodiment 25 is the composition, kit, or method of any one of embodiments 21-23, wherein the first tcdC primary probe oligomer comprises the sequence of SEQ ID NO: 122, with up to two mismatches.

Embodiment 26 is the composition, kit, or method of any one of embodiments 21-23, wherein the first tcdC primary probe oligomer comprises the sequence of SEQ ID NO: 185, with up to two mismatches.

Embodiment 27 is the composition, kit, or method of any one of embodiments 21-26, wherein the first tcdC primary probe oligomer has no mismatches to the 1, 2, 3, 4, 5, 6, 7, 8, 9, or 10 5′-terminal nucleotides of SEQ ID NO: 93-137 or 184-187.

Embodiment 28 is the composition, kit, or method of embodiment 27, wherein the first tcdC primary probe oligomer comprises the sequence of any one of SEQ ID NOs: 93-137 or 184-187.

Embodiment 29 is the composition, kit, or method of any one of embodiments 21-28, wherein the first tcdC primary probe oligomer comprises the sequence of any one of SEQ ID NOs: 138-182 or 188-191, with up to two mismatches.

Embodiment 30 is the composition, kit, or method of embodiment 29, wherein the first tcdC primary probe oligomer comprises the sequence of any one of SEQ ID NOs: 138-182 or 188-191.

Embodiment 31 is the method of any one of embodiments 3 or 5-30, wherein the method is configured to generate a positive signal in the presence of 184T mutant tcdC.

Embodiment 32 is the composition, kit, or method of any one of the preceding embodiments, wherein the second tcdC detection oligomer is present and is configured to specifically hybridize to the sequence of a third tcdC sequence at a site comprising position 184 thereof, but exhibits distinguishably different hybridization to the sequence of a fourth tcdC sequence, wherein the third tcdC sequence is the sequence of SEQ ID NO: 5 and the fourth tcdC sequence is the sequence of SEQ ID NO: 2.

Embodiment 33 is the composition, kit, or method of embodiment 32, wherein the second tcdC detection oligomer comprises the sequence of any one of SEQ ID NOs: 194-196 or 205, with up to two mismatches.

Embodiment 34 is the composition, kit, or method of embodiment 32, wherein the second tcdC detection oligomer comprises the sequence of SEQ ID NO: 196, with up to two mismatches.

Embodiment 35 is the composition, kit, or method of embodiment 32, wherein the second tcdC detection oligomer comprises the sequence of SEQ ID NO: 205, with up to two mismatches.

Embodiment 36 is the composition, kit, or method of any one of embodiments 32-35, wherein the second tcdC detection oligomer has no mismatches to the 1, 2, 3, 4, 5, 6, 7, 8, 9, or 10 3′-terminal nucleotides of SEQ ID NO: 194-196 or 205.

Embodiment 37 is the composition, kit, or method of embodiment 36, wherein the second tcdC detection oligomer comprises the sequence of any one of SEQ ID NOs: 194-196 or 205.

Embodiment 38 is the composition, kit, or method of any one of embodiments 32-37, wherein the composition or kit further comprises at least one second tcdC primary probe oligomer that is configured to form an invasive cleavage structure in the presence of the second tcdC detection oligomer and a polynucleotide comprising the sequence of SEQ ID NO: 5.

Embodiment 39 is the composition, kit, or method of embodiment 38, wherein the second tcdC primary probe oligomer comprises the sequence of any one of SEQ ID NOs: 197-200, 206-212, or 233-237, with up to two mismatches.

Embodiment 40 is the composition, kit, or method of embodiment 38 or 39, wherein the second tcdC primary probe oligomer competes for hybridization to a tcdC nucleic acid under stringent conditions with an oligomer having a sequence consisting of SEQ ID NO: 201-204, 213-219, or 238-242.

Embodiment 41 is the composition, kit, or method of embodiment 39 or 40, wherein the second tcdC primary probe oligomer comprises the sequence of SEQ ID NO: 200, with up to two mismatches.

Embodiment 42 is the composition, kit, or method of embodiment 39 or 40, wherein the second tcdC primary probe oligomer comprises the sequence of SEQ ID NO: 209, with up to two mismatches.

Embodiment 43 is the composition, kit, or method of any one of embodiments 38-42, wherein the second tcdC primary probe oligomer has no mismatches to the 1, 2, 3, 4, 5, 6, 7, 8, 9, or 10 5′-terminal nucleotides of SEQ ID NO: 197-200, 206-212, or 233-237.

Embodiment 44 is the composition, kit, or method of embodiment 43, wherein the second tcdC primary probe oligomer comprises the sequence of any one of SEQ ID NOs: 197-200, 206-212, or 233-237.

Embodiment 45 is the composition, kit, or method of any one of embodiments 38-44, wherein the second tcdC primary probe oligomer comprises the sequence of any one of SEQ ID NOs: 201-204, 213-219, or 238-242 with up to two mismatches.

Embodiment 46 is the composition, kit, or method of embodiment 45, wherein the second tcdC primary probe oligomer comprises the sequence of any one of SEQ ID NOs: 201-204, 213-219, or 238-242.

Embodiment 47 is the method of embodiment 3 or 5, wherein the method is configured not to generate a positive signal in the presence of 117T 184C tcdC.

Embodiment 48 is the method of embodiment 47, wherein the method is configured not to generate a positive signal in the presence of 117D 184C tcdC.

Embodiment 49 is the composition, kit, or method of any one of embodiments 4-48, wherein the composition or kit further comprises at least forward and reverse tcdC amplification oligomers, wherein the forward and reverse tcdC amplification oligomers are configured to produce a tcdC amplicon comprising position 117 of SEQ ID NO: 3 or SEQ ID NO: 4 if the first tcdC detection oligomer is present and/or position 184 of SEQ ID NO: 5 or SEQ ID NO: 2 if the second tcdC detection oligomer is present.

Embodiment 50 is the composition, kit, or method of any one of embodiments 4-48, wherein the first detection oligomer is an iPrimer and the composition or kit further comprises a first additional amplification oligomer; wherein the iPrimer is a forward amplification oligomer and the first additional amplification oligomer is a reverse amplification oligomer, or the iPrimer is a reverse amplification oligomer and the first additional amplification oligomer is a forward amplification oligomer; and wherein the first additional amplification oligomer and first detection oligomer are configured to produce a tcdC amplicon comprising position 117 of SEQ ID NO: 3 or SEQ ID NO: 4.

Embodiment 51 is the composition, kit, or method of any one of embodiments 4-48, wherein the second detection oligomer is an iPrimer and the composition or kit further comprises a second additional amplification oligomer; wherein the iPrimer is a forward amplification oligomer and the second additional amplification oligomer is a reverse amplification oligomer, or the iPrimer is a reverse amplification oligomer and the second additional amplification oligomer is a forward amplification oligomer; and wherein the second additional amplification oligomer and second detection oligomer are configured to produce a tcdC amplicon comprising position 184 of SEQ ID NO: 5 or SEQ ID NO: 2.

Embodiment 52 is the composition, kit, or method of any one of embodiments 1-3 or 49-51, wherein the forward tcdC amplification oligomer comprises the sequence of any one of SEQ ID NOs: 243-248, with up to two mismatches.

Embodiment 53 is the composition, kit, or method of any one of embodiments 1-3 or 49-52, wherein the forward tcdC amplification oligomer competes for hybridization to a tcdC nucleic acid under stringent conditions with an oligomer having a sequence consisting of SEQ ID NO: 243-248.

Embodiment 54 is the composition, kit, or method of embodiment 52 or 53, wherein the forward tcdC amplification oligomer comprises the sequence of SEQ ID NO: 245, with up to two mismatches.

Embodiment 55 is the composition, kit, or method of embodiment 52 or 53, wherein the forward tcdC amplification oligomer comprises the sequence of SEQ ID NO: 247, with up to two mismatches.

Embodiment 56 is the composition, kit, or method of any one of embodiments 1-3 or 49-55, wherein the forward tcdC amplification oligomer has no mismatches to the 1, 2, 3, 4, 5, 6, 7, 8, 9, or 10 3′-terminal nucleotides of any one of SEQ ID NO: 243-248.

Embodiment 57 is the composition, kit, or method of embodiment 56, wherein the forward tcdC amplification oligomer comprises the sequence of any one of SEQ ID NOs: 243-248.

Embodiment 58 is the composition, kit, or method of any one of embodiments 1-3 or 49-57, wherein the reverse tcdC amplification oligomer comprising the sequence of any one of SEQ ID NOs: 249-259, with up to two mismatches.

Embodiment 59 is the composition, kit, or method of any one of embodiments 1-3 or 49-58, wherein the reverse tcdC amplification oligomer competes for hybridization to a tcdC nucleic acid under stringent conditions with an oligomer having a sequence consisting of SEQ ID NO: 249-259.

Embodiment 60 is the composition, kit, or method of embodiment 58 or 59, wherein the reverse tcdC amplification oligomer comprises the sequence of SEQ ID NO: 255, with up to two mismatches.

Embodiment 61 is the composition, kit, or method of embodiment 58 or 59, wherein the reverse tcdC amplification oligomer comprises the sequence of SEQ ID NO: 258, with up to two mismatches.

Embodiment 62 is the composition, kit, or method of any one of embodiments 1-3 or 49-61, wherein the reverse tcdC amplification oligomer has no mismatches to the 1, 2, 3, 4, 5, 6, 7, 8, 9, or 10 3′-terminal nucleotides of any one of SEQ ID NO: 249-259.

Embodiment 63 is the composition, kit, or method of embodiment 62, wherein the reverse tcdC amplification oligomer comprises the sequence of any one of SEQ ID NOs: 249-259.

Embodiment 64 is the composition, kit, or method of any one of embodiments 1 or 3-63, wherein the composition or kit further comprises at least forward and reverse tcdA amplification oligomers, and at least one tcdA detection oligomer, wherein:

the forward and reverse tcdA amplification oligomers are configured to produce a tcdA amplicon having a size of from 80 to 400 nucleotides; and

the tcdA detection oligomer is configured to specifically hybridize to the tcdA amplicon.

Embodiment 65 is the method of embodiment 64, further comprising detecting the presence of a C. difficile pathogenic locus based at least in part on whether the tcdA detection oligomer hybridized to the tcdA amplicon.

Embodiment 66 is the composition, kit, or method of any one of embodiments 2-65, wherein the forward or reverse tcdA amplification oligomer is an iPrimer and the tcdA detection oligomer is a primary probe.

Embodiment 67 is the composition, kit, or method of any one of embodiments 2-65, wherein the tcdA detection oligomer is a primary probe and the composition or kit further comprises an invader oligomer.

Embodiment 68 is the composition, kit, or method of any one of embodiments 2-67, wherein the forward tcdA amplification oligomer comprises the sequence of any one of SEQ ID NOs: 59-60, 64, or 65, with up to two mismatches.

Embodiment 69 is the composition, kit, or method of any one of embodiments 2-68, wherein the forward tcdA amplification oligomer competes for hybridization to a tcdA nucleic acid under stringent conditions with an oligomer having a sequence consisting of SEQ ID NO: 59-60, 64, or 65.

Embodiment 70 is the composition, kit, or method of embodiment 68 or 69, wherein the forward tcdA amplification oligomer comprises the sequence of SEQ ID NO: 64, with up to two mismatches.

Embodiment 71 is the composition, kit, or method of embodiment 68 or 69, wherein the forward tcdA amplification oligomer comprises the sequence of SEQ ID NO: 65, with up to two mismatches.

Embodiment 72 is the composition, kit, or method of any one of embodiments 2-71, wherein the forward tcdA amplification oligomer has no mismatches to the 1, 2, 3, 4, 5, 6, 7, 8, 9, or 10 3′-terminal nucleotides of any one of SEQ ID NO: 59-60, 64, or 65.

Embodiment 73 is the composition, kit, or method of embodiment 72, wherein the forward tcdA amplification oligomer comprises the sequence of any one of SEQ ID NOs: 59-60, 64, or 65.

Embodiment 74 is the composition, kit, or method of any one of embodiments 2-73, wherein the reverse tcdA amplification oligomer comprises the sequence of any one of SEQ ID NOs: 70-73, with up to two mismatches.

Embodiment 75 is the composition, kit, or method of any one of embodiments 2-74, wherein the reverse tcdA amplification oligomer competes for hybridization to a tcdA nucleic acid under stringent conditions with an oligomer having a sequence consisting of SEQ ID NO: 70-73.

Embodiment 76 is the composition, kit, or method of embodiment 74 or 75, wherein the reverse tcdA amplification oligomer comprises the sequence of SEQ ID NO: 70, with up to two mismatches.

Embodiment 77 is the composition, kit, or method of embodiment 74 or 75, wherein the reverse tcdA amplification oligomer comprises the sequence of SEQ ID NO: 72, with up to two mismatches.

Embodiment 78 is the composition, kit, or method of any one of embodiments 2-77, wherein the reverse tcdA amplification oligomer has no mismatches to the 1, 2, 3, 4, 5, 6, 7, 8, 9, or 10 3′-terminal nucleotides of any one of SEQ ID NO: 70-73.

Embodiment 79 is the composition, kit, or method of embodiment 78, wherein the reverse tcdA amplification oligomer comprises the sequence of any one of SEQ ID NOs: 70-73.

Embodiment 80 is the composition, kit, or method of any one of embodiments 2-93, wherein the tcdA detection oligomer comprises the sequence of any one of SEQ ID NOs: 57, 61, 66, or 67, with up to two mismatches.

Embodiment 81 is the composition, kit, or method of any one of embodiments 2-80, wherein the tcdA detection oligomer competes for hybridization to a tcdA nucleic acid under stringent conditions with an oligomer having a sequence consisting of SEQ ID NO: 58, 62, 68, or 69.

Embodiment 82 is the composition, kit, or method of embodiment 80 or 81, wherein the tcdA detection oligomer comprises the sequence of SEQ ID NO: 66, with up to two mismatches.

Embodiment 83 is the composition, kit, or method of embodiment 80 or 81, wherein the tcdA detection oligomer comprises the sequence of SEQ ID NO: 67, with up to two mismatches.

Embodiment 84 is the composition, kit, or method of any one of embodiments 2-83, wherein the tcdA detection oligomer has no mismatches to the 1, 2, 3, 4, 5, 6, 7, 8, 9, or 10 5′-terminal nucleotides of any one of SEQ ID NO: 57, 61, 66, or 67.

Embodiment 85 is the composition, kit, or method of embodiment 84, wherein the tcdA detection oligomer comprises the sequence of any one of SEQ ID NOs: 57, 61, 66, or 67.

Embodiment 86 is the composition, kit, or method of embodiment 84, wherein the tcdA detection oligomer comprises the sequence of any one of SEQ ID NOs: 58, 62, 68, or 69.

Embodiment 87 is the composition, kit, or method of any one of embodiments 2-86, wherein the composition or kit further comprises a tcdA invader oligomer, wherein the tcdA invader oligomer competes for hybridization to a tcdA nucleic acid under stringent conditions with an oligomer having a sequence consisting of SEQ ID NO: 63, and/or the tcdA invader oligomer comprises the sequence of SEQ ID NO: 63, with up to two mismatches.

Embodiment 88 is the composition, kit, or method of embodiment 87, wherein the tcdA invader oligomer has no mismatches to the 1, 2, 3, 4, 5, 6, 7, 8, 9, or 10 3′-terminal nucleotides of any one of SEQ ID NO: 63.

Embodiment 89 is the composition, kit, or method of any one of the preceding embodiments, wherein the composition or kit further comprises forward and reverse tcdB amplification oligomers, wherein: the forward and reverse tcdB amplification oligomers are configured to produce a tcdB amplicon having a size of from 80 to 400 nucleotides; and the tcdB detection oligomer is configured to specifically hybridize to the tcdB amplicon.

Embodiment 90 is a composition or kit comprising a forward tcdB amplification oligomer, an additional forward tcdB amplification oligomer, and a reverse amplification oligomer, wherein the forward tcdB amplification oligomer competes for hybridization to a tcdB nucleic acid under stringent conditions with an oligomer having a sequence consisting of SEQ ID NO: 260-279 or 339 and/or the forward tcdB amplification oligomer comprises the sequence of any one of SEQ ID NOs: 260-279 or 339 with up to two mismatches; wherein the additional forward tcdB amplification oligomer competes for hybridization to a tcdB nucleic acid under stringent conditions with an oligomer having a sequence consisting of SEQ ID NO: 260-279 or 339 and/or the additional forward tcdB amplification oligomer comprises the sequence of any one of SEQ ID NOs: 260-279 or 339 with up to two mismatches; wherein the additional forward tcdB amplification oligomer is different from the forward tcdB amplification oligomer; and wherein the forward tcdB amplification oligomer, additional forward tcdB amplification oligomer, and reverse amplification oligomer are collectively configured to produce a tcdB amplicon having a size of from 80 to 400 nucleotides.

Embodiment 91 is the composition, kit, or method of embodiment 89, wherein the forward and reverse tcdB amplification oligomers are as recited in embodiment 90 and the composition or kit further comprises the additional forward tcdB amplification oligomer as recited in embodiment 90.

Embodiment 92 is a method of detecting tcdB, comprising preparing a composition according to any one of embodiments 89-91, which further comprises a sample comprising or suspected of comprising C. difficile nucleic acid; subjecting the composition to amplification conditions; and detecting the presence of at least one tcdB amplicon.

Embodiment 93 is the composition, kit, or method of any one of embodiments 89-92, wherein the composition or kit further comprises a tcdB detection oligomer configured to specifically hybridize to at least one tcdB amplicon.

Embodiment 94 is the method of embodiment 93, further comprising detecting the presence of a C. difficile pathogenic locus based at least in part on whether the tcdB detection oligomer hybridized to the tcdB amplicon.

Embodiment 95 is the composition, kit, or method of any one of embodiments 89-94, wherein the forward or reverse tcdB amplification oligomer is an iPrimer and the tcdB detection oligomer is a primary probe.

Embodiment 96 is the composition, kit, or method of any one of embodiments 89-95, wherein the tcdB detection oligomer is a primary probe and the composition or kit further comprises an invader oligomer.

Embodiment 97 is the composition, kit, or method of any one of embodiments 89-96, wherein the forward tcdB amplification oligomer comprises the sequence of any one of SEQ ID NOs: 260-279 or 339, with up to two mismatches.

Embodiment 98 is the composition, kit, or method of any one of embodiments 89-97, wherein the forward tcdB amplification oligomer competes for hybridization to a tcdB nucleic acid under stringent conditions with an oligomer having a sequence consisting of SEQ ID NO: 260-279 or 339.

Embodiment 99 is the composition, kit, or method of embodiment 97 or 98, wherein the forward tcdB amplification oligomer comprises the sequence of SEQ ID NO: 274, with up to two mismatches.

Embodiment 100 is the composition, kit, or method of embodiment 97 or 98, wherein the forward tcdB amplification oligomer comprises the sequence of SEQ ID NO: 277, with up to two mismatches.

Embodiment 101 is the composition, kit, or method of embodiment 97 or 98, wherein the forward tcdB amplification oligomer comprises the sequence of SEQ ID NO: 279, with up to two mismatches.

Embodiment 102 is the composition, kit, or method of embodiment 97 or 98, wherein the forward tcdB amplification oligomer comprises the sequence of SEQ ID NO: 339, with up to two mismatches.

Embodiment 103 is the composition, kit, or method of any one of embodiments 89-102, wherein the forward tcdB amplification oligomer has no mismatches to the 1, 2, 3, 4, 5, 6, 7, 8, 9, or 10 3′-terminal nucleotides of any one of SEQ ID NO: 260-279 or 339.

Embodiment 104 is the composition, kit, or method of embodiment 103, wherein the forward tcdB amplification oligomer comprises the sequence of any one of SEQ ID NOs: 260-279 or 339.

Embodiment 105 is the composition, kit, or method of any one of embodiments 90-104, wherein the additional forward tcdB amplification oligomer is present and comprises the sequence of any one of SEQ ID NOs: 274, 277, 279, or 339.

Embodiment 106 is the composition, kit, or method of any one of embodiments 89-105, wherein the reverse tcdB amplification oligomer comprises the sequence of any one of SEQ ID NOs: 317-338, with up to two mismatches.

Embodiment 107 is the composition, kit, or method of any one of embodiments 89-106, wherein the reverse tcdB amplification oligomer competes for hybridization to a tcdB nucleic acid under stringent conditions with an oligomer having a sequence consisting of SEQ ID NO: 317-338.

Embodiment 108 is the composition, kit, or method of embodiment 106 or 107, wherein the reverse tcdB amplification oligomer comprises the sequence of SEQ ID NO: 317, with up to two mismatches.

Embodiment 109 is the composition, kit, or method of embodiment 106 or 107, wherein the reverse tcdB amplification oligomer comprises the sequence of SEQ ID NO: 324 or 325, with up to two mismatches, optionally wherein the composition or kit comprises at least two reverse tcdB amplification oligomers comprising the sequences of SEQ ID NO: 324 with up to two mismatches and of SEQ ID NO: 325 with up to two mismatches, respectively.

Embodiment 110 is the composition, kit, or method of embodiment 106 or 107, wherein the reverse tcdB amplification oligomer comprises the sequence of SEQ ID NO: 336, with up to two mismatches.

Embodiment 111 is the composition, kit, or method of embodiment 106 or 107, wherein the reverse tcdB amplification oligomer comprises the sequence of SEQ ID NO: 338, with up to two mismatches.

Embodiment 112 is the composition, kit, or method of any one of embodiments 89-111, wherein at least one or at least two reverse tcdB amplification oligomers have no mismatches to the 1, 2, 3, 4, 5, 6, 7, 8, 9, or 10 3′-terminal nucleotides of any one of SEQ ID NO: 317-338.

Embodiment 113 is the composition, kit, or method of embodiment 112, wherein at least one or at least two reverse tcdB amplification oligomers comprise the sequence of any one of SEQ ID NOs: 317-338.

Embodiment 114 is the composition, kit, or method of any one of embodiments 93-113, wherein the tcdB detection oligomer comprises the sequence of any one of SEQ ID NOs: 281-298, with up to two mismatches.

Embodiment 115 is the composition, kit, or method of any one of embodiments 93-114, wherein the tcdB detection oligomer competes for hybridization to a tcdB nucleic acid under stringent conditions with an oligomer having a sequence consisting of SEQ ID NO: 299-316.

Embodiment 116 is the composition, kit, or method of embodiment 114 or 115, wherein the tcdB detection oligomer comprises the sequence of SEQ ID NO: 285, with up to two mismatches.

Embodiment 117 is the composition, kit, or method of embodiment 114 or 115, wherein the tcdB detection oligomer comprises the sequence of SEQ ID NO: 287, with up to two mismatches.

Embodiment 118 is the composition, kit, or method of embodiment 114 or 115, wherein the tcdB detection oligomer comprises the sequence of SEQ ID NO: 288, with up to two mismatches.

Embodiment 119 is the composition, kit, or method of any one of embodiments 93-118, wherein the tcdB detection oligomer has no mismatches to the 1, 2, 3, 4, 5, 6, 7, 8, 9, or 10 5′-terminal nucleotides of any one of SEQ ID NO: 281-298.

Embodiment 120 is the composition, kit, or method of embodiment 119, wherein the tcdB detection oligomer comprises the sequence of any one of SEQ ID NOs: 281-298.

Embodiment 121 is the composition, kit, or method of embodiment 119, wherein the tcdB detection oligomer comprises the sequence of any one of SEQ ID NOs: 299-316.

Embodiment 122 is the composition, kit, or method of any one of embodiments 93-121, wherein the composition or kit further comprises a tcdB invader oligomer, wherein the tcdB invader oligomer competes for hybridization to a tcdB nucleic acid under stringent conditions with an oligomer having a sequence consisting of SEQ ID NO: 280 and/or the tcdB invader oligomer comprises the sequence of SEQ ID NO: 280, with up to two mismatches.

Embodiment 123 is the composition, kit, or method of embodiment 122, wherein the tcdB invader oligomer has no mismatches to the 1, 2, 3, 4, 5, 6, 7, 8, 9, or 10 3′-terminal nucleotides of any one of SEQ ID NO: 280.

Embodiment 124 is the composition, kit, or method of any one of the preceding embodiments, wherein the composition or kit further comprises forward and reverse cdtB amplification oligomers, and at least one cdtB detection oligomer, wherein: the forward and reverse cdtB amplification oligomers are configured to produce a cdtB amplicon having a size of from 80 to 400 nucleotides; and the cdtB detection oligomer is configured to specifically hybridize to the cdtB amplicon.

Embodiment 125 is the method of embodiment 124, further comprising detecting the presence of a C. difficile Cdt locus based at least in part on whether the cdtB detection oligomer hybridized to the cdtB amplicon.

Embodiment 126 is the composition, kit, or method of embodiment 124 or 125, wherein the forward or reverse cdtB amplification oligomer is an iPrimer and the cdtB detection oligomer is a primary probe.

Embodiment 127 is the composition, kit, or method of embodiment 124 or 125, wherein the cdtB detection oligomer is a primary probe and the composition or kit further comprises an invader oligomer.

Embodiment 128 is the composition, kit, or method of any one of embodiments 124-127, wherein the composition or kit comprises a forward cdtB amplification oligomer comprising the sequence of any one of SEQ ID NOs: 30-33 or 55, with up to two mismatches.

Embodiment 129 is the composition, kit, or method of any one of embodiments 124-128, wherein the forward cdtB amplification oligomer competes for hybridization to a cdtB nucleic acid under stringent conditions with an oligomer having a sequence consisting of SEQ ID NO: 30-33 or 55.

Embodiment 130 is the composition, kit, or method of embodiment 128 or 129, wherein the forward cdtB amplification oligomer comprises the sequence of SEQ ID NO: 33, with up to two mismatches.

Embodiment 131 is the composition, kit, or method of embodiment 128 or 129, wherein the forward cd/B amplification oligomer comprises the sequence of SEQ ID NO: 31, with up to two mismatches.

Embodiment 132 is the composition, kit, or method of any one of embodiments 124-131, wherein the forward cd/B amplification oligomer has no mismatches to the 1, 2, 3, 4, 5, 6, 7, 8, 9, or 10 3′-terminal nucleotides of any one of SEQ ID NO: 30-33 or 55.

Embodiment 133 is the composition, kit, or method of embodiment 132, wherein the forward cdtB amplification oligomer comprises the sequence of any one of SEQ ID NOs: 30-33 or 55.

Embodiment 134 is the composition, kit, or method of any one of embodiments 124-133, wherein the composition or kit comprises a reverse cd/B amplification oligomer comprising the sequence of any one of SEQ ID NOs: 40-41, 49-50, or 56, with up to two mismatches.

Embodiment 135 is the composition, kit, or method of any one of embodiments 124-134, wherein the reverse cdtB amplification oligomer competes for hybridization to a cd/B nucleic acid under stringent conditions with an oligomer having a sequence consisting of SEQ ID NO: 40-41, 49-50, or 56.

Embodiment 136 is the composition, kit, or method of embodiment 134 or 135, wherein the reverse cdtB amplification oligomer comprises the sequence of SEQ ID NO: 40, with up to two mismatches.

Embodiment 137 is the composition, kit, or method of embodiment 134 or 135, wherein the reverse cdtB amplification oligomer comprises the sequence of SEQ ID NO: 41, with up to two mismatches.

Embodiment 138 is the composition, kit, or method of embodiment 134 or 135, wherein the reverse cdtB amplification oligomer comprises the sequence of SEQ ID NO: 56, with up to two mismatches.

Embodiment 139 is the composition, kit, or method of any one of embodiments 124-138, wherein at least one or at least two reverse cd/B amplification oligomers have no mismatches to the 1, 2, 3, 4, 5, 6, 7, 8, 9, or 10 3′-terminal nucleotides of any one of SEQ ID NO: 40-41, 49, 50, or 56.

Embodiment 140 is the composition, kit, or method of embodiment 139, wherein the composition or kit comprises a reverse cd/B amplification oligomer comprising the sequence of any one of SEQ ID NOs: 40-41, 49, 50, or 56.

Embodiment 141 is the composition, kit, or method of any one of embodiments 124-140, wherein the cdtB detection oligomer comprises the sequence of any one of SEQ ID NOs: 34-36, 43-45, 51, or 52, with up to two mismatches.

Embodiment 142 is the composition, kit, or method of any one of embodiments 124-141, wherein the cd/B detection oligomer competes for hybridization to a cdtB nucleic acid under stringent conditions with an oligomer having a sequence consisting of SEQ ID NO: 37-39, 46-48, 53, or 54.

Embodiment 143 is the composition, kit, or method of embodiment 141 or 142, wherein the cd/B detection oligomer comprises the sequence of SEQ ID NO: 34, with up to two mismatches.

Embodiment 144 is the composition, kit, or method of embodiment 141 or 142, wherein the cdtB detection oligomer comprises the sequence of SEQ ID NO: 35, with up to two mismatches.

Embodiment 145 is the composition, kit, or method of embodiment 141 or 142, wherein the cdtB detection oligomer comprises the sequence of SEQ ID NO: 51, with up to two mismatches.

Embodiment 146 is the composition, kit, or method of any one of embodiments 124-145, wherein the cd/B detection oligomer has no mismatches to the 1, 2, 3, 4, 5, 6, 7, 8, 9, or 10 5′-terminal nucleotides of any one of SEQ ID NO: 34-36, 43-45, 51, or 52.

Embodiment 147 is the composition, kit, or method of embodiment 146, wherein the cd/B detection oligomer comprises the sequence of any one of SEQ ID NOs: 34-36, 43-45, 51, or 52.

Embodiment 148 is the composition, kit, or method of embodiment 146, wherein the cd/B detection oligomer comprises the sequence of any one of SEQ ID NOs: 37-39, 46-48, 53, or 54.

Embodiment 149 is the composition, kit, or method of any one of embodiments 124-148, wherein the composition or kit further comprises a cd/B invader oligomer, wherein the cdtB invader oligomer competes for hybridization to a cd/B nucleic acid under stringent conditions with an oligomer having a sequence consisting of SEQ ID NO: 42, and/or the cdtB invader oligomer comprises the sequence of SEQ ID NO: 42 with up to two mismatches.

Embodiment 150 is the composition, kit, or method of embodiment 149, wherein the cd/B invader oligomer has no mismatches to the 1, 2, 3, 4, 5, 6, 7, 8, 9, or 10 3′-terminal nucleotides of SEQ ID NO: 42.

Embodiment 151 is the composition, kit, or method of any one of the preceding embodiments, wherein the composition or kit further comprises forward and reverse cdtA amplification oligomers, and at least one cdtA detection oligomer, wherein:

the forward and reverse cdtA amplification oligomers are configured to produce a cdtA amplicon having a size of from 80 to 400 nucleotides; and

the cdtA detection oligomer is configured to specifically hybridize to the cdtA amplicon.

Embodiment 152 is the method of embodiment 151, further comprising detecting the presence of a C. difficile Cdt locus based at least in part on whether the cdtA detection oligomer hybridized to the cdtA amplicon.

Embodiment 153 is the composition, kit, or method of embodiment 151 or 152, wherein the forward or reverse cdtA amplification oligomer is an iPrimer and the cdtA detection oligomer is a primary probe.

Embodiment 154 is the composition, kit, or method of embodiment 151 or 152, wherein the cdtA detection oligomer is a primary probe and the composition or kit further comprises an invader oligomer.

Embodiment 155 is the composition, kit, or method of any one of embodiments 151-154, wherein the composition or kit comprises a forward cdtA amplification oligomer comprising the sequence of SEQ ID NO: 13, with up to two mismatches.

Embodiment 156 is the composition, kit, or method of any one of embodiments 151-155, wherein the forward cdtA amplification oligomer competes for hybridization to a cdtA nucleic acid under stringent conditions with an oligomer having a sequence consisting of SEQ ID NO: 13.

Embodiment 157 is the composition, kit, or method of any one of embodiments 151-156, wherein the forward cdtA amplification oligomer has no mismatches to the 1, 2, 3, 4, 5, 6, 7, 8, 9, or 10 3′-terminal nucleotides of any one of SEQ ID NO: 13, or comprises the sequence of SEQ ID NO: 13.

Embodiment 158 is the composition, kit, or method of any one of embodiments 151-157, wherein the composition or kit comprises a reverse cdtA amplification oligomer comprising the sequence of any one of SEQ ID NOs: 21-29, with up to two mismatches.

Embodiment 159 is the composition, kit, or method of any one of embodiments 151-158, wherein the reverse cdtA amplification oligomer competes for hybridization to a cdtA nucleic acid under stringent conditions with an oligomer having a sequence consisting of SEQ ID NO: 21-29.

Embodiment 160 is the composition, kit, or method of any one of embodiments 151-159, wherein at least one or at least two reverse cdtA amplification oligomers have no mismatches to the 1, 2, 3, 4, 5, 6, 7, 8, 9, or 10 3′-terminal nucleotides of any one of SEQ ID NO: 21-29.

Embodiment 161 is the composition, kit, or method of any one of embodiments 151-160, wherein the composition or kit comprises a cdtA detection oligomer comprising the sequence of any one of SEQ ID NOs: 16 or 17, with up to two mismatches.

Embodiment 162 is the composition, kit, or method of any one of embodiments 151-161, wherein the cdtA detection oligomer competes for hybridization to a cdtA nucleic acid under stringent conditions with an oligomer having a sequence consisting of SEQ ID NO: 18-20.

Embodiment 163 is the composition, kit, or method of embodiment 161 or 162, wherein the cdtA detection oligomer comprises the sequence of SEQ ID NO: 16, with up to two mismatches.

Embodiment 164 is the composition, kit, or method of embodiment 161 or 162, wherein the cdtA detection oligomer comprises the sequence of SEQ ID NO: 17, with up to two mismatches.

Embodiment 165 is the composition, kit, or method of any one of embodiments 151-164, wherein the cdtA detection oligomer has no mismatches to the 1, 2, 3, 4, 5, 6, 7, 8, 9, or 10 5′-terminal nucleotides of any one of SEQ ID NO: 16 or 17.

Embodiment 166 is the composition, kit, or method of embodiment 165, wherein the cdtA detection oligomer comprises the sequence of any one of SEQ ID NOs: 16 or 17.

Embodiment 167 is the composition, kit, or method of embodiment 165, wherein the cdtA detection oligomer comprises the sequence of any one of SEQ ID NOs: 18-20.

Embodiment 168 is the composition, kit, or method of any one of embodiments 151-167, wherein the composition or kit further comprises a cdtA invader oligomer, wherein the cdtA invader oligomer competes for hybridization to a cdtA nucleic acid under stringent conditions with an oligomer having a sequence consisting of SEQ ID NO: 14 or 15, and/or the cdtA invader oligomer comprises the sequence of SEQ ID NO: 14 or 15 with up to two mismatches.

Embodiment 169 is the composition, kit, or method of embodiment 168, wherein the cdtA invader oligomer has no mismatches to the 1, 2, 3, 4, 5, 6, 7, 8, 9, or 10 3′-terminal nucleotides of any one of SEQ ID NO: 14 or 15.

Embodiment 170 is a detection oligomer comprising the sequence set forth in positions 7-18 of any one of SEQ ID NOs: 18-20, 37-39, 46-48, 53, 54, 58, 62, 68, 69, 138-182, 188-191, 201-204, 213-219, 238-242, or 299-316, wherein the detection oligomer further comprises sufficient additional sequence to specifically hybridize to a C. difficile tcdA, tcdB, tcdC, cdtA, or cdtB nucleic acid.

Embodiment 171 is the detection oligomer of embodiment 170, which is configured to specifically hybridize to the reverse complement of the sequence set forth in any one of SEQ ID NOs: 16, 17, 34-36, 43-45, 51, 52, 57, 61, 66, 67, 93-137, 184-187, 197-200, 206-212, 233-237, or 281-298.

Embodiment 172 is the detection oligomer of embodiment 170 or 171, comprising the sequence set forth in any one of SEQ ID NOs: 16, 17, 34-36, 43-45, 51, 52, 57, 61, 66, 67, 93-137, 184-187, 197-200, 206-212, 233-237, or 281-298 with up to two mismatches.

Embodiment 173 is the detection oligomer of embodiment 172, comprising the sequence set forth in any one of SEQ ID NOs: 16, 17, 34-36, 43-45, 51, 52, 57, 61, 66, 67, 93-137, 184-187, 197-200, 206-212, 233-237, or 281-298.

Embodiment 174 is the detection oligomer of any one of embodiments 170-173, comprising the sequence set forth in any one of SEQ ID NOs: 18-20, 37-39, 46-48, 53, 54, 58, 62, 68, 69, 138-182, 188-191, 201-204, 213-219, 238-242, or 299-316 with up to two mismatches.

Embodiment 175 is the detection oligomer of embodiment 174, comprising the sequence set forth in any one of SEQ ID NOs: 18-20, 37-39, 46-48, 53, 54, 58, 62, 68, 69, 138-182,188-191, 201-204, 213-219, 238-242, or 299-316.

Embodiment 176 is the composition, kit, method, or detection oligomer of any one of embodiments 1-89 or 93-175, wherein at least one detection oligomer is non-extendable.

Embodiment 177 is the composition, kit, method, or detection oligomer of any one of embodiments 1-89 or 93-176, wherein at least one detection oligomer comprises a label.

Embodiment 178 is the composition, kit, method, or detection oligomer of any one of embodiments 1-89 or 93-177, wherein at least one detection oligomer has a length of 15 to 55 nucleotides.

Embodiment 179 is a composition or kit comprising at least one detection oligomer of any one of embodiments 170-178 and at least one secondary detection oligomer, wherein the secondary detection oligomer comprises at least one label and is configured to interact with a fragment of the detection oligomer.

Embodiment 180 is the composition, kit, or method of any one of embodiments 1-89 or 93-169, wherein the composition or kit further comprises at least one secondary detection oligomer that comprises a label and is configured to interact with a fragment of a detection oligomer.

Embodiment 181 is the composition, kit, or method of embodiment 179 or 180, wherein the secondary detection oligomer comprises at least two labels.

Embodiment 182 is the composition, kit, or method of embodiment 181, wherein the at least two labels include a FRET pair.

Embodiment 183 is the composition, kit, or method of embodiment 181 or 182, wherein the at least two labels include a quencher.

Embodiment 184 is the composition, kit, or method of any one of embodiments 179-183, wherein the fragment of the detection oligomer is a 5′-terminal flap of at least six nucleotides.

Embodiment 185 is the composition, kit, or method of embodiment 184, wherein the one or more secondary detection oligomers are FRET cassettes.

Embodiment 186 is the composition, kit, or method of any one of embodiments 180-184, wherein the kit or composition comprises at least a tcdB primary detection oligomer as recited in any one of embodiments 89, 93-96, or 114-123, and a tcdA primary detection oligomer as recited in any one of embodiments 2 or 80-86, and the one or more secondary detection oligomers include a secondary detection oligomer configured to generate a positive signal in the presence of a tcdB nucleic acid and in the presence of a tcdA nucleic acid.

Embodiment 187 is the composition, kit, or method of any one of embodiments 180-186, wherein the kit or composition comprises at least a first tcdC primary probe oligomer as recited in any one of embodiments 21-30 and a second tcdC primary probe oligomer as recited in any one of embodiments 38-46, and the one or more secondary detection oligomers include a secondary detection oligomer configured to generate a positive signal in the presence of a tcdC allele comprising a 117del mutation and in the presence of a tcdC allele comprising a 184T mutation.

Embodiment 188 is the composition, kit, or method of any one of embodiments 180-187, wherein the one or more secondary detection oligomers include a secondary detection oligomer comprising the sequence of SEQ ID NO: 10.

Embodiment 189 is the composition, kit, or method of any one of embodiments 180-188, wherein the one or more secondary detection oligomers include a secondary detection oligomer comprising the sequence of SEQ ID NO: 11.

Embodiment 190 is the composition, kit, or method of any one of embodiments 180-189, wherein the one or more secondary detection oligomers include a secondary detection oligomer comprising the sequence of SEQ ID NO: 12.

Embodiment 191 is the composition, kit, or method of any one of embodiments 1-169 or 176-190, wherein the composition or kit comprises a nuclease with structure-specific activity toward a three-strand structure formed by 3′-end invasion.

Embodiment 192 is the composition, kit, or method of any one of embodiments 1-169 or 176-191, wherein the composition or kit comprises a cleavase or 5′-nuclease.

Embodiment 193 is the composition, kit, or method of any one of embodiments 1-169 or 176-192, wherein the composition or kit comprises a FEN1 nuclease.

Embodiment 194 is the composition, kit, or method of any one of embodiments 1-169 or 176-193, wherein the composition or kit comprises a polymerase.

Embodiment 195 is the composition, kit, or method of any one of embodiments 1-169 or 176-194, wherein the composition or kit comprises a DNA polymerase.

Embodiment 196 is the composition, kit, or method of any one of embodiments 1-169 or 176-195, wherein the composition or kit comprises a thermostable DNA polymerase.

Embodiment 197 is the composition, kit, or method of embodiment 196, wherein the thermostable DNA polymerase is a hot-start DNA polymerase.

Embodiment 198 is the composition, kit, or method of any one of embodiments 1-169 or 176-197, wherein the composition or kit comprises NTPs.

Embodiment 199 is the composition, kit, or method of any one of embodiments 1-169 or 176-198, wherein composition or kit comprises deoxyribonucleotide triphosphates.

Embodiment 200 is a method of detecting at least one C. difficile nucleic acid comprising:

preparing a composition according to any one of embodiments 176-199, or comprising at least one detection oligomer of any one of embodiments 170-175, which further comprises a sample comprising or suspected of comprising C. difficile nucleic acid or at least one C. difficile amplicon;

detecting the presence of the C. difficile nucleic acid or the C. difficile amplicon by performing a hybridization assay; and determining whether the detection oligomer hybridized to the C. difficile nucleic acid or the C. difficile amplicon.

Embodiment 201 is the method of embodiment 200, wherein the composition comprises at least one secondary detection oligomer as recited in any one of embodiments 179-190, and the method comprises determining whether the detection oligomer hybridized to the C. difficile nucleic acid or the C. difficile amplicon at least in part by exposing the detection oligomer to a structure-specific nuclease and determining whether a fragment of the detection oligomer produced by the structure-specific nuclease interacts with the secondary detection oligomer.

Embodiment 202 is the method of embodiment 201, wherein the fragment of the detection oligomer is a 5′-terminal flap.

Embodiment 203 is the method of any one of embodiments 200-202, wherein the composition further comprises at least one invasive oligomer that hybridizes to a site in the C. difficile nucleic acid or the C. difficile amplicon that overlaps the hybridization site of the detection oligomer and, in the presence of the detection oligomer and the C. difficile nucleic acid or the C. difficile amplicon, forms a structure recognized for cleavage by the structure-specific nuclease.

Embodiment 204 is the method of embodiment 203, wherein the invasive oligomer competes for hybridization to the C. difficile nucleic acid or the C. difficile amplicon under stringent conditions with an oligomer having a sequence consisting of the sequence of any one of SEQ ID NOs: 14, 15, 40-42, 63-65, 74-76, 86-92, 183, 192-196, 205, 220, 221, 223-225, 260-280, or 317.

Embodiment 205 is the method of embodiment 217 or 218, wherein the invasive oligomer has a sequence comprising the sequence of any one of SEQ ID NOs: 14, 15, 40-42, 63-65, 74-76, 86-92, 183, 192-196, 205, 220, 221, 223-225, 260-280, or 317 with up to two mismatches.

Embodiment 206 is the composition, kit, detection oligomer, or method of any one of the preceding embodiments, wherein at least one oligomer comprises at least one methylated cytosine.

Embodiment 207 is the composition, kit, detection oligomer, or method of any one of the preceding embodiments, wherein the sequences of SEQ ID NOs include adenine methylation as indicated in the Table of Sequences.

Embodiment 208 is the composition, kit, detection oligomer, or method of any one of the preceding embodiments, wherein the sequences of SEQ ID NOs include cytosine methylation as indicated in the Table of Sequences.

Embodiment 209 is a composition of any one of embodiments 1-2, 4, 10-30, 32-46, 49-64, 66-91, 93, 95-124, 126-151, 153-169, 179-199, or 206-208, or comprising a detection oligomer of any one of embodiments 170-175, which is aqueous, frozen, or lyophilized, or wherein at least one oligomer is bound to a solid substrate.

Embodiment 210 is the kit of any one of embodiments 1-2, 4, 10-30, 32-46, 49-64, 66-91, 93, 95-124, 126-151, 153-169, 179-199, or 206-209, further comprising instructions for detecting at least one of a C. difficile tcdA, tcdB, tcdC, cdtA, or cdtB nucleic acid in a sample.

Embodiment 211 is the use of a composition or kit of any one of embodiments 1-2, 4, 10-30, 32-46, 49-64, 66-91, 93, 95-124, 126-151, 153-169, 179-199, or 206-210, or of a detection oligomer of any one of embodiments 170-175 for detecting at least one of a C. difficile tcdA, tcdB, tcdC, cdtA, or cdtB nucleic acid in a sample.

Section headings are provided for the convenience of the reader and are not to be interpreted to limit the scope of the disclosure.

### DETAILED DESCRIPTION OF CERTAIN EMBODIMENTS

### Overview

Oligomers for amplifying and/or detecting tcdA, tcdB, tcdC, cdtA, and cdtB target sequences of C. difficile are provided herein. In some embodiments, oligomers for detecting two or more of these genes can be used in a multiplex format so that a single reaction provides information about multiple genes. Certain oligomers can be used to specifically detect tcdC alleles associated with hypervirulence. In some embodiments, additional amplification oligomers or probes are used, e.g., to increase sensitivity or mitigate false negatives that may result from certain sequence polymorphisms.

In some embodiments, the oligomers are for use in performing invasive cleavage assays such as INVADER or INVADER PLUS assays. Such approaches can use a set of detection oligomers, comprising invasive and primary probe oligomers, to form a structure in which the 3′ end of an invasive oligomer competes with the 5′ flap of another oligomer for hybridization to the target sequence, resulting in a complex comprising a partially single-stranded primary probe. A cleavage agent is used that recognizes this structure and cleaves the primary probe, thus liberating the 5′ flap for subsequent detection; this is referred to as an invasive cleavage reaction. In some embodiments, detection occurs by a second invasive cleavage reaction wherein the 5′ flap hybridizes with a double-labeled oligomer (e.g., FRET cassette) and forms a substrate for the cleavage agent. Cleavage of the double-labeled oligomer can separate the two labels and thereby provide a detectable change in label behavior, such as a loss of quenching of a fluorophore. It has been found that this approach is suitable for discrimination of hypervirulent from non-hypervirulent tcdC alleles and also for multiplexing, facilitating a rapid and streamlined approach for assessing whether toxin-producing C. difficile nucleic acid is in a sample, whether the sample contains a tcdC allele associated with hypervirulence, and/or whether it has one, the other, or both of the Pathogenic locus and the Cdt locus.

### Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references, and expressions such as “one or more items” include singular references unless the context clearly dictates otherwise. Thus, for example, reference to “an oligomer” includes a plurality of oligomers and the like; in a further example, a statement that “one or more secondary detection oligomers are FRET cassettes” includes a situation in which there is exactly one secondary detection oligomer and it is a FRET cassette. The conjunction “or” is to be interpreted in the inclusive sense, i.e., as equivalent to “and/or,” unless the inclusive sense would be unreasonable in the context. When “at least one” member of a class (e.g., oligomer) is present, reference to “the” member (e.g., oligomer) refers to the present member (if only one) or at least one of the members (e.g., oligomers) present (if more than one).

Measured and measureable values are understood to be approximate, taking into account significant digits and the error associated with the measurement. All ranges are to be interpreted as encompassing the endpoints in the absence of express exclusions such as “not including the endpoints”; thus, for example, “within 10-15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. “Consisting essentially of” means that additional component(s), composition(s) or method step(s) that do not materially change the basic and novel characteristics of the compositions and methods described herein can be included in those compositions or methods. Such characteristics include the ability to detect a toxigenic C. difficile nucleic acid sequence present in a sample with specificity that distinguishes the toxigenic C. difficile nucleic acid from other Clostridium species and other known pathogens. In some embodiments, the characteristics include the ability to detect a C. difficile nucleic acid sequence at a sensitivity sufficient to detect as little as about 10 CFU (Colony Forming Units)/mL of toxigenic C. difficile. In some embodiments, the characteristics include the ability to detect a C. difficile nucleic acid sequence within about 60 minutes and/or within about 50 cycles from the beginning of an amplification reaction when a cycled amplification reaction is used.

Where a claim to a composition or kit recites an oligomer with a first given feature (e.g., competing for hybridization under stringent conditions for binding to a nucleic acid with an oligomer having a sequence consisting of a given sequence) and claims dependent thereon recite an oligomer with additional given features (e.g., comprising the sequence of at least one of a given list of SEQ ID NOs), then the composition or kit can comprise a plurality of oligomers that collectively have the features or an individual oligomer with the first and additional features (including but not necessarily limited to when the features are related; e.g., competing for hybridization under stringent conditions for binding to a nucleic acid with an oligomer having a sequence consisting of a given sequence would be related to comprising the sequence of a SEQ ID NO that overlaps with the given sequence).

A “sample” refers to material that may contain nucleic acid from C. difficile, including but not limited to biological, clinical, environmental, and food samples. Environmental samples include environmental material such as surface matter, soil, water and industrial samples, as well as samples obtained from food and dairy processing instruments, apparatus, equipment, utensils, disposable and non-disposable items. “Biological” or “clinical” samples refer to a tissue or material derived from a living or dead human or animal which may contain a C. difficile target nucleic acid, including, for example, body fluids, fecal samples and swabs thereof, and colon or rectal biopsies or washes. A sample can be treated to physically, chemically, or mechanically disrupt tissue or cell structure to release intracellular nucleic acids into a solution which may contain enzymes, buffers, salts, detergents and the like, to prepare the sample for analysis. These examples are not to be construed as limiting the sample types applicable to the present disclosure.

“Nucleic acid” and “polynucleotide” refer to a multimeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., inosine or others; see The Biochemistry of the Nucleic Acids 5-36, Adams et al., ed., 11^{th} ed., 1992), derivatives of purines or pyrimidines (e.g., N⁴-methyl guanine, N⁶-methyladenine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Embodiments of oligomers that can affect stability of a hybridization complex include PNA oligomers, oligomers that include 2′-methoxy or 2′-fluoro substituted RNA, or oligomers that affect the overall charge, charge density, or steric associations of a hybridization complex, including oligomers that contain charged linkages (e.g., phosphorothioates) or neutral groups (e.g., methylphosphonates). Methylated cytosines such as 5-methylcytosines can be used in conjunction with any of the foregoing backbones/sugars/linkages including RNA or DNA backbones (or mixtures thereof) unless otherwise indicated. RNA and DNA equivalents have different sugar moieties (i.e., ribose versus deoxyribose) and can differ by the presence of uracil in RNA and thymine in DNA. The differences between RNA and DNA equivalents do not contribute to differences in homology because the equivalents have the same degree of complementarity to a particular sequence. It is understood that when referring to ranges for the length of an oligonucleotide, amplicon, or other nucleic acid, that the range is inclusive of all whole numbers (e.g., 19-25 contiguous nucleotides in length includes 19, 20, 21, 22, 23, 24, and 25).

“C residues” include methylated and unmethylated cytosines unless the context indicates otherwise. In some embodiments, methylated cytosines comprise or consist of 5-methylcytosines.

“A residues” include methylated and unmethylated adenines unless the context indicates otherwise. In some embodiments, methylated adenines comprise or consist of 2′-O-methyladenosine.

“Nucleic acid amplification” or simply “amplification” refers to any in vitro procedure that produces multiple copies of a target nucleic acid sequence, or its complementary sequence, or fragments thereof (i.e., an amplified sequence containing less than the complete target nucleic acid). Amplification methods include, for example, replicase-mediated amplification, polymerase chain reaction (PCR), ligase chain reaction (LCR), strand-displacement amplification (SDA), helicase-dependent amplification, and transcription-mediated amplification (TMA), also known as transcription-associated amplification. Replicase-mediated amplification uses self-replicating RNA molecules, and a replicase such as QB-replicase (see. e.g., U.S. Pat. No. 4,786,600, incorporated by reference herein). PCR amplification uses a DNA polymerase, pairs of primers, and thermal cycling to synthesize multiple copies of two complementary strands of dsDNA or from a cDNA (see. e.g., U.S. Pat. Nos. 4,683,195; 4,683,202; and 4,800,159; each incorporated by reference herein). LCR amplification uses four or more different oligonucleotides to amplify a target and its complementary strand by using multiple cycles of hybridization, ligation, and denaturation (see. e.g., U.S. Pat. Nos. 5,427,930 and 5,516,663, each incorporated by reference herein). SDA uses a primer that contains a recognition site for a restriction endonuclease and an endonuclease that nicks one strand of a hemimodified DNA duplex that includes the target sequence, whereby amplification occurs in a series of primer extension and strand displacement steps (see, e.g., U.S. Pat. Nos. 5,422,252; 5,547,861; and 5,648,211; each incorporated by reference herein). Helicase-dependent amplification uses a helicase to separate the two strands of a DNA duplex generating single-stranded templates, followed by hybridization of sequence-specific primers hybridize to the templates and extension by DNA polymerase to amplify the target sequence (see, e.g., U.S. Pat. No. 7,282,328, incorporated by reference herein). Amplification may be linear or exponential.

Transcription associated amplification uses a DNA polymerase, an RNA polymerase, deoxyribonucleoside triphosphates, ribonucleoside triphosphates, a promoter-containing oligonucleotide, and optionally can include other oligonucleotides, to ultimately produce multiple RNA transcripts from a nucleic acid template (described in detail in U.S. Pat. Nos. 5,399,491 and 5,554,516, Kacian et al., U.S. Pat. No. 5,437,990, Burg et al., PCT Nos. WO 88/01302 and WO 88/10315, Gingeras et al., U.S. Pat. No. 5,130,238, Malek et al., U.S. Pat. Nos. 4,868,105 and 5,124,246, Urdea et al., PCT No. WO 94/03472, McDonough et al., PCT No. WO 95/03430, and Ryder et al.). Methods that use TMA are described in detail previously (U.S. Pat. Nos. 5,399,491 and 5,554,516).

In cyclic amplification methods that detect amplicons in real-time, the term “Threshold cycle” (Ct) is a measure of the emergence time of a signal associated with amplification of target, and may, for example, be approximately 10× standard deviation of the normalized reporter signal. Once an amplification reaches the “threshold cycle,” generally there is considered to be a positive amplification product of a sequence to which the probe binds. Binding of the probe generally provides substantial information the identity of the product (e.g., that it is a tcdB or tcdA amplicon, as the case may be, or a member of a certain class of alleles of a gene in the case of one or more allele-specific probe(s)). The amplification product can additionally be further characterized through methods known to one of skill in the art, such as gel electrophoresis, nucleic acid sequencing, and other such analytical procedures.

An “oligomer” or “oligonucleotide” refers to a nucleic acid of generally less than 1,000 nucleotides (nt), including those in a size range having a lower limit of about 2 to 5 nt and an upper limit of about 500 to 900 nt. Some particular embodiments are oligomers in a size range with a lower limit of about 5 to 15, 16, 17, 18, 19, or 20 nt and an upper limit of about 50 to 600 nt, and other particular embodiments are in a size range with a lower limit of about 10 to 20 nt and an upper limit of about 22 to 100 nt. Oligomers can be purified from naturally occurring sources, but can be synthesized by using any well known enzymatic or chemical method. Oligomers can be referred to by a functional name (e.g., capture probe, primer or promoter primer) but those skilled in the art will understand that such terms refer to oligomers. Oligomers can form secondary and tertiary structures by self-hybridizing or by hybridizing to other polynucleotides. Such structures can include, but are not limited to, duplexes, hairpins, cruciforms, bends, and triplexes. Oligomers may be generated in any manner, including chemical synthesis, DNA replication, reverse transcription, PCR, or a combination thereof. In some embodiments, oligomers that form invasive cleavage structures are generated in a reaction (e.g., by extension of a primer in an enzymatic extension reaction).

By “amplicon” or “amplification product” is meant a nucleic acid molecule generated in a nucleic acid amplification reaction and which is derived from a target nucleic acid. An amplicon or amplification product contains a target nucleic acid sequence that can be of the same or opposite sense as the target nucleic acid. In some embodiments, an amplicon has a length of about 100-2000 nucleotides, about 100-1500 nucleotides, about 100-1000 nucleotides, about 100-800 nucleotides, about 100-700 nucleotides, about 100-600 nucleotides, or about 100-500 nucleotides.

An “amplification oligonucleotide” or “amplification oligomer” refers to an oligonucleotide that hybridizes to a target nucleic acid, or its complement, and participates in a nucleic acid amplification reaction, e.g., serving as a primer and/or promoter-primer. Particular amplification oligomers contain at least 10 contiguous bases, and optionally at least 11, 12, 13, 14, 15, 16, 17, 18, 19, or 20 contiguous bases, that are complementary to a region of the target nucleic acid sequence or its complementary strand. The contiguous bases can be at least 80%, at least 90%, or completely complementary to the target sequence to which the amplification oligomer binds. In some embodiments, an amplification oligomer comprises an intervening linker or non-complementary sequence between two segments of complementary sequence, e.g., wherein the two complementary segments of the oligomer collectively comprise at least 10 complementary bases, and optionally at least 11, 12, 13, 14, 15, 16, 17, 18, 19, or 20 complementary bases. One skilled in the art will understand that the recited ranges include all whole and rational numbers within the range (e.g., 92% or 98.377%). Particular amplification oligomers are 10 to 60 bases long and optionally can include modified nucleotides.

A “primer” refers to an oligomer that hybridizes to a template nucleic acid and has a 3′ end that is extended by polymerization. A primer can be optionally modified, e.g., by including a 5′ region that is non-complementary to the target sequence. Such modification can include functional additions, such as tags, promoters, or other sequences used or useful for manipulating or amplifying the primer or target oligonucleotide. A primer modified with a 5′ promoter sequence can be referred to as a “promoter-primer.” A person of ordinary skill in the art of molecular biology or biochemistry will understand that an oligomer that can function as a primer can be modified to include a 5′ promoter sequence and then function as a promoter-primer, and, similarly, any promoter-primer can serve as a primer with or without its 5′ promoter sequence.

“Detection oligomer” or “probe” refers to an oligomer that interacts with a target nucleic acid to form a detectable complex. Examples include invasive probes (also referred to as invader oligomers), iPrimers, and primary probes, discussed below. A probe's target sequence generally refers to the specific sequence within a larger sequence (e.g., gene, amplicon, locus, etc.) to which the probe hybridizes specifically. A detection oligomer can include target-specific sequences and a non-target-complementary sequence. Such non-target-complementary sequences can include sequences which will confer a desired secondary or tertiary structure, such as a flap or hairpin structure, which can be used to facilitate detection and/or amplification (e.g., U.S. Pat. Nos. 5,118,801, 5,312,728, 6,835,542, 6,849,412, 5,846,717, 5,985,557, 5,994,069, 6,001,567, 6,913,881, 6,090,543, and 7,482,127; WO 97/27214; WO 98/42873; Lyamichev et al., Nat. Biotech., 17:292 (1999); and Hall et al., PNAS, USA, 97:8272 (2000)). Probes of a defined sequence can be produced by techniques known to those of ordinary skill in the art, such as by chemical synthesis, and by in vitro or in vivo expression from recombinant nucleic acid molecules.

By “probe system” is meant a plurality of detection oligomers or probes for detecting a target sequence. In some embodiments, a probe system comprises at least primary and secondary probes, at least invasive and primary probes, or invasive, primary, and secondary probes. An “invasive probe” or “invader oligomer” refers to an oligonucleotide that hybridizes to a target nucleic acid at a location near the region of hybridization between a primary probe and the target nucleic acid, wherein the invasive probe oligonucleotide comprises a portion (e.g., a chemical moiety, or nucleotide, whether complementary to that target or not) that overlaps with the region of hybridization between the primary probe oligonucleotide and the target nucleic acid. An “iPrimer” is an invasive probe that is also an amplification oligomer and undergoes extension by a DNA polymerase—that is, the iPrimer can function as an invasive probe in the absence of extension, and it can also undergo extension during amplification, functioning as a primer. The “primary probe” for an invasive cleavage assay includes a target-specific region that hybridizes to the target nucleic acid, and further includes a “5′ flap” region that is not complementary to the target nucleic acid. In general, detection can either be direct (i.e., probe hybridized directly to the target) or indirect (i.e., involving an intermediate structure that links a detectable label or detectably labeled molecule (e.g., a FRET cassette) to the target). In some embodiments, a primary probe comprises a target-hybridizing sequence and a non-target-complementary sequence. In some embodiments, a primary probe undergoes nucleolysis (e.g., cleavage, such as 5′-cleavage or endonucleolysis) upon hybridization to a target sequence in the presence of an appropriate cleavage agent, which can be a nuclease, such as structure-specific nuclease, e.g., a cleavase or 5′-nuclease. In some embodiments, such nucleolysis results in liberation of a “flap” or cleavage fragment from the primary probe that interacts with the secondary probe. In some embodiments, the secondary probe comprises at least one label. In some embodiments, the secondary probe comprises at least a pair of labels, such as an interacting pair of labels, e.g., a FRET pair or a fluorophore and quencher. In some embodiments, interaction of the secondary probe with a liberated flap of the primary probe results in a detectable change in the emission properties of the second probe, e.g., as discussed below with respect to INVADER® assays, FRET, and/or quenching. In some embodiments, a probe system comprises a primary probe and a secondary probe configured to interact with a liberated flap of the primary probe, e.g., the primary probe can be cleaved to give a liberated flap sufficiently complementary to the secondary probe or a segment thereof to form a complex.

By “hybridization” or “hybridize” is meant the ability of two completely or partially complementary nucleic acid strands to come together under specified hybridization assay conditions in a parallel or antiparallel orientation to form a stable structure having a double-stranded region. The two constituent strands of this double-stranded structure, sometimes called a hybrid, are held together by hydrogen bonds. Although these hydrogen bonds most commonly form between nucleotides containing the bases adenine and thymine or uracil (A and T or U) or cytosine and guanine (C and G) on single nucleic acid strands, base pairing can also form between bases which are not members of these “canonical” pairs. Non-canonical base pairing is well-known in the art. (See, e.g., R. L. P. Adams et al., The Biochemistry of the Nucleic Acids (11th ed. 1992).)

As used herein, “specific” means pertaining to only one (or to only a particularly indicated group), such as having a particular effect on only one (or on only a particularly indicated group), or affecting only one (or only a particularly indicated group) in a particular way. For example, a cleaved 5′ flap specific for a FRET cassette will be able to hybridize to that FRET cassette, form an invasive cleavage structure, and promote a cleavage reaction, but will not be able to hybridize to a different FRET cassette (e.g., a FRET cassette having a different 5′ flap-hybridizing sequence) to promote a cleavage reaction. In addition, specific may be used in relation to a combination of oligonucleotides, such as a set of amplification and detection oligonucleotides (e.g., a amplification oligonucleotides may amplify multiple target sequences non-specifically but the detection oligonucleotides will only detect a specific amplified sequence, thus making the combination specific).

As used herein, the term “specifically hybridizes” means that under given hybridization conditions a probe or primer detectably hybridizes substantially only to its target sequence(s) in a sample comprising the target sequence(s) (i.e., there is little or no detectable hybridization to non-targeted sequences). Notably, for example in the case of various polymorphic C. difficile toxin and toxin-related gene sequences, an oligomer or combination thereof can be configured to specifically hybridize to any one of a set of targets. Thus, an oligomer described as specifically hybridizing to a first tcdC allele can also (but does not necessarily) specifically hybridize to a second (or a second and third, etc.) tcdC allele. Amplification and detection oligomers that specifically hybridize to a target nucleic acid are useful to amplify and detect target nucleic acids, but not non-targeted nucleic acids, especially non-targeted nucleic acids of phylogenetically closely related organisms such as Clostridia other than C. difficile or in some cases alleles that give rise to a phenotype different from the phenotype associated with a targeted allele. Thus, the oligomer hybridizes to target nucleic acid to a sufficiently greater extent than to non-target nucleic acid to enable one having ordinary skill in the art to accurately amplify and/or detect the presence (or absence) of nucleic acid derived from the specified target (e.g., toxigenic C. difficile) as appropriate. In general, reducing the degree of complementarity between an oligonucleotide sequence and its target sequence will decrease the degree or rate of hybridization of the oligonucleotide to its target region. However, the inclusion of one or more non-complementary nucleosides or nucleobases may facilitate the ability of an oligonucleotide to discriminate against non-targeted nucleic acid sequences.

Specific hybridization can be measured using techniques known in the art and described herein, such as in the examples provided below. In some embodiments, there is at least a 10-fold difference between target and non-target hybridization signals in a test sample, at least a 100-fold difference, or at least a 1,000-fold difference. In some embodiments, non-target hybridization signals in a test sample are no more than the background signal level.

By “stringent hybridization conditions,” or “stringent conditions” is meant conditions permitting an oligomer to preferentially hybridize to a target nucleic acid (e.g., C. difficile nucleic acid) and not to nucleic acid derived from a closely related non-targeted organisms. While the definition of stringent hybridization conditions does not vary, the actual reaction environment that can be used for stringent hybridization may vary depending upon factors including the GC content and length of the oligomer, the degree of similarity between the oligomer sequence and sequences of targeted and non-targeted nucleic acids that may be present in the test sample. Hybridization conditions include the temperature and the composition of the hybridization reagents or solutions. Exemplary hybridization assay conditions for amplifying and/or detecting target nucleic acids derived from one or more strains of C. difficile with the oligomers of the present disclosure correspond to a temperature of 63° C. to 72° C. or 65° C. to 69° C. when the salt concentration, such as a divalent salt, e.g., MgCl₂, is in the range of 5-21 mM. Additional details of hybridization conditions are set forth in the Examples section. Other acceptable stringent hybridization conditions could be easily ascertained by those having ordinary skill in the art.

“Label” or “detectable label” refers to a moiety or compound joined directly or indirectly to a probe that is detected or leads to a detectable signal. Direct joining can use covalent bonds or non-covalent interactions (e.g., hydrogen bonding, hydrophobic or ionic interactions, and chelate or coordination complex formation) whereas indirect joining can use a bridging moiety or linker (e.g., via an antibody or additional oligonucleotide(s), which amplify a detectable signal. Any detectable moiety can be used, e.g., radionuclide, ligand such as biotin or avidin, enzyme, enzyme substrate, reactive group, chromophore such as a dye or particle (e.g., latex or metal bead) that imparts a detectable color, luminescent compound (e.g. bioluminescent, phosphorescent, or chemiluminescent compound), and fluorescent compound (i.e., fluorophore). Embodiments of fluorophores include those that absorb light (e.g., have a peak absorption wavelength) in the range of 495 to 690 nm and emit light (e.g., have a peak emission wavelength) in the range of 520 to 710 nm, which include those known as FAM™, TET™, HEX, CAL FLUOR™ (Orange or Red), CY, and QUASAR™ compounds. Fluorophores can be used in combination with a quencher molecule that absorbs light when in close proximity to the fluorophore to diminish background fluorescence. Such quenchers are well known in the art and include, e.g., BLACK HOLE QUENCHER™ (or BHQ™), Blackberry Quencher® (or BBQ650®), Eclipse®, or TAMRA™ compounds. Particular embodiments include a “homogeneous detectable label” that is detectable in a homogeneous system in which bound labeled probe in a mixture exhibits a detectable change compared to unbound labeled probe, which allows the label to be detected without physically removing hybridized from unhybridized labeled probe (e.g., U.S. Pat. Nos. 5,283,174, 5,656,207, and 5,658,737). Exemplary homogeneous detectable labels include chemiluminescent compounds, including acridinium ester (“AE”) compounds, such as standard AE or AE derivatives which are well known (U.S. Pat. Nos. 5,656,207, 5,658,737, and 5,639,604). Methods of synthesizing labels, attaching labels to nucleic acid, and detecting signals from labels are known (e.g., Sambrook et al., Molecular Cloning, A Laboratory Manual, 2nd ed. (Cold Spring Harbor Laboratory Press, Cold Spring Harbor, NY, 1989) at Chapt. 10, and U.S. Pat. Nos. 5,658,737, 5,656,207, 5,547,842, 5,283,174, 5,585,481, 5,639,604, and 4,581,333, and EP Pat. App. 0 747 706). Other detectably labeled probes include FRET cassettes, TaqMan™ probes, molecular torches, and molecular beacons. FRET cassettes are discussed in detail below. TaqMan™ probes include a donor and acceptor label wherein fluorescence is detected upon enzymatically degrading the probe during amplification in order to release the fluorophore from the presence of the quencher. Molecular torches and beacons exist in open and closed configurations wherein the closed configuration quenches the fluorophore and the open position separates the fluorophore from the quencher to allow a change in detectable fluorescent signal. Hybridization to target opens the otherwise closed probes.

Sequences are “sufficiently complementary” if they allow stable hybridization of two nucleic acid sequences, e.g., stable hybrids of probe and target sequences, although the sequences need not be completely complementary. That is, a “sufficiently complementary” sequence that hybridizes to another sequence by hydrogen bonding between a subset series of complementary nucleotides by using standard base pairing (e.g., G:C, A:T, or A:U), although the two sequences can contain one or more residues (including abasic positions) that are not complementary so long as the entire sequences in appropriate hybridization conditions to form a stable hybridization complex. Sufficiently complementary sequences can be at least 80%, at least 90%, or completely complementary in the sequences that hybridize together. Appropriate hybridization conditions are well known to those skilled in the art, can be predicted based on sequence composition, or can be determined empirically by using routine testing (e.g., Sambrook et al., Molecular Cloning, A Laboratory Manual, 2^{nd} ed. at §§ 1.90-1.91, 7.37-7.57, 9.47-9.51 and 11.47-11.57, particularly §§ 9.50-9.51, 11.12-11.13, 11.45-11.47 and 11.55-11.57).

A “non-extendable” oligomer includes a blocking moiety at or near its 3′-terminus to prevent extension. A blocking group near the 3′ end is in some embodiments within five residues of the 3′ end and is sufficiently large to limit binding of a polymerase to the oligomer, and other embodiments contain a blocking group covalently attached to the 3′ terminus. Many different chemical groups can be used to block the 3′ end, e.g., alkyl groups, non-nucleotide linkers, alkane-diol dideoxynucleotide residues (e.g., 3′-hexanediol residues), and cordycepin. Further examples of blocking moieties include a 3′-deoxy nucleotide (e.g., a 2′,3′-dideoxy nucleotide); a 3′-phosphorylated nucleotide; a fluorophore, quencher, or other label that interferes with extension; an inverted nucleotide (e.g., linked to the preceding nucleotide through a 3′-to-3′ phosphodiester, optionally with an exposed 5′-OH or phosphate); or a protein or peptide joined to the oligonucleotide so as to prevent further extension of a nascent nucleic acid chain by a polymerase. A non-extendable oligonucleotide of the present disclosure can be at least 10 bases in length, and can be up to 15, 20, 25, 30, 35, 40, 50 or more nucleotides in length. Non-extendable oligonucleotides that comprise a detectable label can be used as probes.

References, particularly in the claims, to “the sequence of SEQ ID NO: X” refer to the base sequence of the corresponding sequence listing entry and do not require identity of the backbone (e.g., RNA, 2′-O-Me RNA, or DNA) or base modifications (e.g., methylation of cytosine residues) unless otherwise indicated. Furthermore, T residues are understood to be interchangeable with U residues, and vice versa, unless otherwise indicated.

Unless otherwise indicated, “forward,” “sense,” “positive-sense,” or “positive-strand” nucleic acid generally refers to the coding strand of an ORF (open reading frame) or non-coding nucleic acid on the same strand as the coding strand of the transcript, operon, mRNA, etc. of which it is a part, or otherwise of the closest ORF, and “reverse,” “antisense,” “negative-sense,” “negative-strand” nucleic acid refers to the complement of a “sense,” “positive-sense,” or “positive-strand” nucleic acid. Exemplary sense-strand sequences are provided in SEQ ID NOs: 1-7 in the Sequence Table below. Unless otherwise indicated, “hybridizing to a nucleic acid” and the like includes hybridizing to either a sense or antisense strand thereof, e.g., either strand of a dsDNA sequence. Similarly, expressions such as “hybridization to a site comprising position X of SEQ ID NO: Y” and “competing for hybridization to SEQ ID NO: Y” can generally include hybridizing to either a sense or antisense strand of SEQ ID NO: Y; where a hybridized oligomer is configured to produce an amplicon, the proper orientation will be immediately apparent to one skilled in the art.

As used herein, the term “invasive cleavage structure” (or simply “cleavage structure”) refers to a structure comprising: (1) a target nucleic acid, (2) an upstream nucleic acid (e.g., an invasive probe oligonucleotide), and (3) a downstream nucleic acid (e.g., a primary probe oligonucleotide), where the upstream and downstream nucleic acids anneal to contiguous regions of the target nucleic acid, and where an overlap forms between the a 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether or not the overlapping base(s) of the upstream nucleic acid are complementary with the target nucleic acid, and whether or not those bases are natural bases or non-natural bases. In some embodiments, the 3′ portion of the upstream nucleic acid that overlaps with the downstream duplex is a non-base chemical moiety such as an aromatic ring structure, as disclosed, for example, in U.S. Pat. No. 6,090,543. In some embodiments, one or more of the nucleic acids may be attached to each other, for example through a covalent linkage such as nucleic acid stem-loop, or through a non-nucleic acid chemical linkage (e.g., a multi-carbon chain). An invasive cleavage structure also is created when a cleaved 5′ flap hybridizes to a FRET cassette (i.e., when the “target nucleic acid” and the “downstream nucleic acid” are covalently linked in a stem-loop configuration). The “target nucleic acid” sequence of a FRET cassette that hybridizes to a cleaved 5′ flap can be referred to as a “5′ flap-hybridizing sequence.”

As used herein, an “INVADER assay” or “invasive cleavage assay” refers to an assay for detecting target nucleic acid sequences in which an invasive cleavage structure is formed and cleaved in the presence of the target sequence. In some embodiments, reagents for an invasive cleavage assay include: a cleavage agent; and oligonucleotides (e.g., an “invasive probe,” a “primary probe,” and a “FRET cassette”). In some embodiments the invasive probe is an amplification oligomer or extension product thereof. The invasive cleavage assay can combine two invasive signal amplification reactions (i.e., a “primary reaction” and a “secondary reaction”) in series in a single reaction mixture. In some embodiments, detecting the presence of an invasive cleavage structure is achieved using a cleavage agent. The primary probe can be part of a probe system. In some embodiments, an additional portion of the primary probe comprises or consists of a 3′ terminal nucleotide which is not complementary to the target nucleic acid and/or which is non-extendable. In some embodiments, an additional portion of the primary probe is configured to interact with a FRET cassette, e.g., comprises a FRET cassette interacting-sequence, e.g., which is not complementary to the target nucleic acid. In some embodiments, the reagents for an INVADER assay further comprise a nuclease, e.g., a cleavase, e.g., a FEN enzyme (e.g., Afu, Ave, RAD2 or XPG proteins) or other enzyme (e.g., a DNA polymerase with 5′ nuclease activity, optionally with inactivated or reduced synthetic activity) wherein the nuclease has activity specific for a structure formed when both the invasive and primary probes are hybridized to a target sequence (e.g., a structure that can result when a duplex of the primary probe and the target undergoes 3′-end invasion by the invasive probe, wherein at least the 3′ end and/or an intermediate portion of the invasive probe is hybridized, the 5′ end of the primary probe is free, and an intermediate and/or 3′-terminal portion of the primary probe is hybridized). In some embodiments, the reagents for an INVADER assay further comprise a buffer solution. In some embodiments, the buffer solution comprises a source of divalent cations (e.g., Mn2+ and/or Mg2+ ions, such as a magnesium salt or manganese salt, e.g., MgCl₂, MnCl₂, magnesium acetate, manganese acetate, etc.). In some embodiments, the reagents for an INVADER assay further comprise at least one third oligomer, such as at least one amplification oligomer that together with the first oligomer is configured to produce an amplicon, e.g., via PCR. In such embodiments the primary probe can comprise a target-hybridizing sequence configured to specifically hybridize to the amplicon. In some embodiments, the reagents for an INVADER assay further comprise amplification reagents, such as PCR reagents. Embodiments of an INVADER assay in which the target sequence is amplified can be referred to as INVADER PLUS assays. Including amplification in the assay can provide a lower limit of detection. INVADER assays, cleavases, other nucleases, other possible INVADER/INVADER PLUS® reagents, etc., are discussed, for example, in U.S. Pat. Nos. 5,846,717, 5,985,557, 5,994,069, 6,001,567, 6,913,881, 6,090,543, 7,482,127, and 9,096,893; WO 97/27214; WO 98/42873; Lyamichev et al., Nat. Biotech., 17:292 (1999); Hall et al., PNAS, USA, 97:8272 (2000); and WO 2016/179093.

As used herein, the term “flap endonuclease” or “FEN” (e.g., “FEN enzyme”) refers to a class of nucleolytic enzymes that act as structure-specific endonucleases on DNA structures with a duplex containing a single-stranded 5′ overhang, or flap, on one of the strands that is displaced by another strand of nucleic acid, such that there are overlapping nucleotides at the junction between the single and double-stranded DNA. FEN enzymes catalyze hydrolytic cleavage of the phosphodiester bond 3′ adjacent to the junction of single and double stranded DNA, releasing the overhang, or “flap” (see Trends Biochem. Sci. 23:331-336 (1998) and Ann. Rev. Biochem. 73: 589-615 (2004)). FEN enzymes may be individual enzymes, multi-subunit enzymes, or may exist as an activity of another enzyme or protein complex, such as a DNA polymerase. A flap endonuclease may be thermostable. Examples of FEN enzymes useful in the methods disclosed herein are described in U.S. Pat. Nos. 5,614,402; 5,795,763; 6,090,606; and in published PCT applications identified by WO 98/23774; WO 02/070755; WO 01/90337; and WO 03/073067, each of which is incorporated by reference in its entirety. Particular examples of commercially available FEN enzymes include the Cleavase® enzymes (Hologic, Inc.).

“Cassette,” when used in reference to an INVADER assay and/or invasive cleavage assay or reaction, as used herein refers to an oligomer or combination of oligomers configured to generate a detectable signal in response to cleavage of a detection oligomer in an INVADER assay. In some embodiments, the cassette hybridizes to an cleavage product (e.g., a “flap”) from cleavage of the detection oligomer (e.g., primary probe). In some embodiments, such hybridization results in a detectable change in fluorescence. In some embodiments, such hybridization forms a second invasive cleavage structure, such that the cassette can then be cleaved. In some embodiments, a cassette comprises an interacting pair of labels, e.g., a FRET pair (in which case the cassette is a “FRET cassette”). In some embodiments, a FRET cassette undergoes a detectable change in fluorescence properties upon hybridization to an cleavage product from cleavage of the detection oligomer. For example, a FRET cassette can increase fluorescence emission at a first wavelength and/or decrease fluorescence emission at a second wavelength based on a change in the average distance between labels upon hybridization to a cleavage product from cleavage of the detection oligomer. This can result from a decrease in energy transfer from a donor fluorophore (e.g., a decrease in quenching of a fluorophore or a decrease in energy transfer from a donor fluorophore to an acceptor fluorophore). In some embodiments, a FRET cassette adopts a hairpin conformation, wherein the interaction of the pair of labels substantially suppresses (e.g., quenches) a detectable energy emission (e.g., a fluorescent emission). In some embodiments, a FRET cassette comprises a portion that hybridizes to a complementary cleaved 5′ flap of a primary probe to form an invasive cleavage structure that is a substrate for a cleavage agent (e.g., FEN enzyme). In some embodiments, cleavage of the FRET cassette by a cleavage agent separates the donor and acceptor moieties with the result of relieving the suppression and permitting generation of a signal.

A “117del tcdC allele” is an allele of C. difficile tcdC in which there is a deletion corresponding to position 117 of the wild-type tcdC open reading frame. Thus, for example, in the exemplary 117del sequence SEQ ID NO: 3, positions 112-121 are ATTTTGGCGT, while in the exemplary wild-type sequence fragment SEQ ID NO: 2, positions 112-122 are ATTTTAGGCGT. Position 117 has been observed to be polymorphic, with G and T known to occur there as well (see SEQ ID NOs: 1 and 4, respectively). Thus, a “117D tcdC allele” is one in which an A, G, or T is present at position 117 (D is the IUPAC ambiguity code for A, G, or T/U), and the individual non-deletion polymorphs are 117A, 117G, and 117T tcdC alleles. One skilled in the art can identify corresponding positions between sequence variants using known alignment methods, such as the Smith-Waterman or Needleman-Wunsch algorithms using standard parameters. Highly similar sequences such as SEQ ID NOs: 1-5 can alternatively be manually aligned.

A “184T tcdC allele” is an allele of C. difficile tcdC in which there is a T at the position corresponding to position 184 of the wild-type tcdC open reading frame. Thus, for example, in the exemplary 184T sequence SEQ ID NO: 5, positions 179-189 are CTAATTAAACA, while in the exemplary wild-type sequence fragment SEQ ID NO: 2, positions 179-189 are CTAACCAAACA. Position 183 is also polymorphic, and 184T and 184C alleles each can be either 183C or 183T (for example, SEQ ID NO: 2 is 183C 184C, SEQ ID NOs: 1 and 4 are 183T 184C, and SEQ ID NO: 5 is 183T 184T). Identification of corresponding positions is as discussed above.

Unless defined otherwise, all scientific and technical terms used herein have the same meaning as commonly understood by those skilled in the relevant art. General definitions can be found in technical books relevant to the art of molecular biology, e.g., DICTIONARY OF MICROBIOLOGY AND MOLECULAR BIOLOGY, 2nd ed. (Singleton et al., 1994, John Wiley & Sons, New York, NY) or THE HARPER COLLINS DICTIONARY OF BIOLOGY (Hale & Marham, 1991, Harper Perennial, New York, NY).

### Exemplary Compositions, Kits, Methods, and Uses

The present disclosure provides oligomers, compositions, and kits, useful for amplifying and detecting C. difficile nucleic acids from a sample.

In some embodiments, oligomers are provided, e.g., in a kit or composition. Oligomers generally comprise a target-hybridizing region, e.g., configured to hybridize specifically to a C. difficile nucleic acid, such as a C. difficile toxin or toxin-related gene, for example, tcdA, tcdB, tcdC, cdtA, or cdtB. While oligomers of different lengths and base composition can be used for amplifying C. difficile nucleic acids, in some embodiments oligomers in this disclosure have target-hybridizing regions from 10-60 bases in length, 12-50 bases in length, 12-40 bases in length, 12-35 bases in length, or 12-30 bases in length. In some embodiments, an oligomer comprises a second region of sequence in addition to the target-hybridizing region, which can be located 5′ of the target-hybridizing region. In some embodiments, an oligomer does not comprise a second region of sequence. In some embodiments, the second region of sequence is a promoter. In some embodiments, the second region of sequence is configured to interact with a FRET cassette.

In some embodiments, a pair of oligomers is provided wherein one oligomer is configured to hybridize to a sense strand of a C. difficile nucleic acid and the other is configured to hybridize to an anti-sense strand of a C. difficile nucleic acid. Such oligomers include primer pairs for PCR or other forms of amplification.

In some embodiments, one or more oligomers, such as a primer set (defined as at least two primers configured to generate an amplicon from a target sequence) or a primer set and an additional oligomer (e.g., detection oligomer) which is optionally non-extendible and/or labeled (e.g., for use as a primary probe or part of a probe system, such as together with a FRET cassette), are configured to hybridize to at least one, two, three, or four C. difficile toxin or toxin-related genes, for example, tcdA, tcdB, tcdC, cdtA, or cdtB. When present, the additional oligomer (e.g., detection oligomer) can be configured to specifically hybridize to an amplicon produced by the primer set.

In some embodiments, a plurality of oligomers are provided which collectively hybridize to tcdA and tcdB; tcdC and tcdA; tcdB and tcdC; tcdC and cdtB; tcdB and cdtB; tcdA and cdtB; tcdA, tcdB, and tcdC; tcdA, tcdB, and cdtB; tcdA, cdtB, and tcdC; cdtB, tcdB, and tcdC; cdtB, tcdA, tcdB, and tcdC; tcdC and cdtA; tcdB and cdtA; tcdA and cdtA; tcdA, tcdB, and tcdC; tcdA, tcdB, and cdtA; tcdA, cdtA, and tcdC; cdtA, tcdB, and tcdC; cdtA, tcdA, tcdB, and tcdC; or cdtA, cdtB, tcdA, tcdB, and tcdC, it being understood that the foregoing gene names refer to C. difficile genes. In some embodiments, a plurality of primer sets is provided which collectively hybridize to any one of the foregoing combinations of genes. In some embodiments, a plurality of primer sets and additional oligomers (e.g., detection oligomers) which are optionally non-extendible and/or labeled (e.g., for use as a primary probe, optionally as part of a probe system, such as together with a FRET cassette) is provided which collectively hybridize to any one of the foregoing combinations of genes.

Exemplary cdtA, cdtB, tcdA, tcdB, and tcdC sequences are provided in the Sequence Table below. Additional exemplary sequences are provided, for example, in GenBank and can be accessed by one skilled in the art.

In some embodiments, one or more oligomers in a set, kit, composition, or reaction mixture comprise a methylated cytosine (e.g., 5-methylcytosine). In some embodiments, at least half of the cytosines in an oligomer are methylated. In some embodiments, all or substantially all (e.g., all but one or two) of the cytosines in an oligomer are methylated. In some embodiments, all or substantially all cytosines are methylated except for one or more cytosines at the 3′ end or within 2, 3, 4, or 5 bases of the 3′ end are unmethylated. Cytosine methylation can be used to modulate the affinity of an oligomer for a target and generally can result in an increased melting temperature for the hybridized complex.

Exemplary oligomer sets (primer pairs and detection oligomers, e.g., to be labeled or used as primary detection oligomers) and probe systems (primary and secondary detection oligomers) are set forth in the following tables. It should be understood that a detection oligomer in Table A, when used as a primary detection oligomer (e.g., with a structure-specific nuclease, such as in an invasive cleavage assay, e.g., an INVADER or INVADER PLUS® assay), can be combined with any secondary detection oligomer associated with that primary detection oligomer in Table B.

TABLE A
Exemplary oligomer sets. Oligomers are referred to by their SEQ ID
NO (see the Sequence Table below). The allele(s) targeted by the exemplary detection
oligomers for tcdC in Table A are indicated in parentheses. The oligomers can include
methylation, labeling, and other non-sequence features noted in the Sequence Table.

| | | |
|---|---|---|
| Oligomer 1 (e.g., | Oligomer 2 (e.g., | Detection Oligomer (optionally |
| forward primer) | reverse primer) | labeled and/or non-extendable, e.g., |
| SEQ ID NO(s) | SEQ ID NO(s) | probe) SEQ ID NO(s) |

For targeting tcdC

| | | |
|---|---|---|
| 243 | one or more of 87-90 | 117del probes: one or more of 138, 141- |
| | or 192-193 (reverse | 171, 175-182 |
| | iPrimers) | |
| 244 | same as above | same as above |
| 245 | same as above | same as above |
| 246 | same as above | same as above |
| 247 | same as above | same as above |
| 248 | same as above | same as above |
| 76 | one or more of 251- | one or more of 188-191 |
| (forward iPrimer) | 259 (one or more of | |
| | 251-255 if detecting | |
| | 184T polymorphism | |
| | in addition to 117del) | |
| 243 | one or more of 251- | 117del probes: one or more of 138, 141- |
| | 259 (one or more of | 171, 175-182 with 86* and/or |
| | 251-255 if detecting | 184T probes: one or more of 201-204 |
| | 184T polymorphism | with one or more of 194*, |
| | in addition to 117del) | 195*, or 196*; or one or more of |
| | | 213, 215, 217, or 218 with 205*; |
| | | or one or more of 202, 204, |
| | | 239, 240, 242 with 223* |
| 244 | same as above | same as above |
| 245 | same as above | same as above |
| 246 | same as above | same as above |
| 247 | same as above | same as above |
| 248 | same as above | same as above |
| 243 | 192 (reverse iPrimer) | 117del probes: one or more of 138, 141- |
| | | 171, 175-182 with 86* |
| 244 | same as above | same as above |
| 245 | same as above | same as above |
| 246 | same as above | same as above |
| 247 | same as above | same as above |
| 248 | same as above | same as above |
| one or more of | 225 (reverse iPrimer) | 184T probes: one or more of 213, 215, |
| 243, 244, 245, 246, | | 217, or 218 |
| 247, or 248 | | |
| same as above | 221 (reverse iPrimer) | 184T probes: one or more of 214, 216, |
| | | or 219 |

For targeting tcdA

| | | |
|---|---|---|
| 59 | one or more of 70, | 62 with 63* |
| | 72, or 73 | |
| 60 | same as above | same as above |
| 64 | same as above | same as above |
| (forward iPrimer) | | |
| 65 | same as above | same as above |
| (forward iPrimer) | | |
| 64 | One or more of 70-73 | one or more of 58, 68, or 69 |
| (forward iPrimer) | | |
| 65 | same as above | same as above |
| (forward iPrimer) | | |

For targeting tcdB

| | | |
|---|---|---|
| 260 | one or more of 318- | one or more of 299-316 (e.g., 305 and at |
| (forward iPrimer) | 220, 322 or 328-338, | least one of 302, 304, or 306) |
| | e.g., 328-334 | |
| 261 | same as above | same as above |
| (forward iPrimer) | | |
| 260 and 261 | same as above | same as above |
| (forward iPrimers) | | |
| 321 | same as above | same as above |
| 260 and 321 | same as above | same as above |
| (forward iPrimers) | | |
| 262 | same as above | same as above |
| (forward iPrimer) | | |
| 263 | same as above | same as above |
| (forward iPrimer) | | |
| 262 and 263 | same as above | same as above |
| (forward iPrimers) | | |
| 264 | one or more of 322 | one or more of 299, 312, or 316 |
| | or 332-338, e.g., 337 | with 280* |
| | and 338 | |
| 265 | same as above | same as above |
| 266 | same as above | same as above |
| 268 | same as above | same as above |
| 269 | same as above | same as above |
| 270 | same as above | same as above |
| 271 | same as above | same as above |
| 272 | same as above | same as above |
| one or more of | 317 (reverse iPrimer) | one or more of 299, 300, |
| 264-279 or 339, | | 308-312, or 316 |
| e.g., a pair listed above | | |
| same as above | 323 (reverse iPrimer) | one or more of 300 or 307 |
| same as above | 324 (reverse iPrimer) | one or more of 299, 300, |
| | | 308-312, or 316 |
| same as above | 325 (reverse iPrimer) | same as above |
| same as above | 324 and 325 (reverse | same as above |
| | iPrimers) | |
| same as above | 324 and 326 (reverse | same as above |
| | iPrimers) | |
| same as above | 326 (reverse iPrimer) | same as above |
| same as above | 336 (reverse iPrimer) | 300-306 or 308-311 |

For targeting cdtA

| | | |
|---|---|---|
| 13 | one or more of 21-29 | one or more of 18-20 with 14* and/or 15* |

For targeting cdtB

| | | |
|---|---|---|
| 30 | one or more of 40 | one or more of 37-39 or 47 |
| (forward iPrimer | and 41 (reverse | |
| if 47 used as probe) | iPrimers if one or | |
| | more of 37-39 used as | |
| | probe) | |
| 31 | same as above | one or more of 37-39 or 46 |
| (forward iPrimer | | |
| if 46 used as probe) | | |
| 32 | same as above | same as above |
| (forward iPrimer | | |
| if 46 used as probe) | | |
| 33 | same as above | same as above |
| (forward iPrimer | | |
| if 46 used as probe) | | |
| 30 | one or more of 49, | one or more of 38 with 42*; or 47; or 48 |
| (forward iPrimer | 50, and 56 | with 42* |
| if 47 used as probe) | | |
| 31 | same as above | one or more of 38 with 42*; or 46, 53, |
| | | or 54; or 48 with 42* |
| 32 | same as above | same as above |
| 33 | same as above | same as above |

*used as an invasive oligomer to form a substrate for a structure-specific nuclease together with indicated detection oligomer

The sets shown above are exemplary and not exclusive. For example, amplification oligomers should be oppositely oriented with convergent 3′ ends, and detection oligomers should hybridize to an amplicon produced by the amplification oligomers (i.e., hybridization should occur in the region between amplification oligomer hybridization sites or in some cases overlapping the 3′ end of an amplification oligomer hybridization site). In some embodiments, at least two forward primers targeting one of the genes referred to in Table A are used in combination. In some embodiments, at least two forward oligomers targeting tcdB are used in combination. Combinations of oligomers with different sequences can provide a benefit to assay sensitivity given the intraspecific heterogeneity of C. difficile sequences, including tcdB. See Example 2 below.

The detection oligomer SEQ ID NOs referred to in Table A (other than invasive oligomers) generally include 5′ flap sequences, which are essentially arbitrary sequences that can interact with a FRET cassette after cleavage by a cleavage agent (see Table B below for exemplary secondary detection oligomers and 5′ flap sequences that interact with them). Target-hybridizing sequences for the detection oligomers, which omit the 5′ flap sequences, are also provided in the Sequence Table, and those skilled in the art will understand that the target hybridizing sequence of one detection oligomer can be paired with the 5′ flap sequence of another detection oligomer or another 5′ flap sequence that provides similar hybridization properties. Additionally, in some embodiments, the 5′-flap sequence may be omitted, such as in assay formats such as TaqMan where a 5′-flap is unnecessary in a detection oligomer. In some embodiments, a detection oligomer, such as a detection oligomer in a combination shown in Table A, is provided with a partially self-complementary sequence in place of the 5′-flap sequence, such as in the case of a molecular torch probe, discussed below.

Regarding tcdC, detection oligomers listed above are configured to detect (generate signal in the presence of) 117del or 184T alleles of tcdC, which are associated with hypervirulence. Thus, methods provided herein can be configured not to generate a positive signal in the presence of 117T 184C tcdC, or 117D 184C tcdC—that is, to the extent any signal is generated at all from the relevant detection system with such a tcdC allele, it is sufficiently low to be considered negative. The 117del primary probe and invasive oligomer (invader or iPrimer) can be configured so that the primary probe target-hybridizing sequence (e.g., SEQ ID NO: 93-137 or 184-187) hybridizes to a site on the opposite side of the deletion from the invasive oligomer, such that the 3′ end of the invasive oligomer is close enough to the 5′-end of the target-hybridizing sequence to form an invasive cleavage structure when both are in a complex with a 117del allele, but not a 117D allele. SEQ ID NOs: 106 (primary probe target hybridizing sequence) and 88 (iPrimer) (in part) are shown below as examples, respectively, aligned with an excerpt of SEQ ID NO: 3, a 117del allele (position indicated with an asterisk), in which the deletion site is indicated with a dash. (As discussed elsewhere herein, the primary probe can have a nonextendable 3′-end to prevent extension.) The 5′-end of the primary probe target-hybridizing sequence and the 3′-end of the invasive oligomer (here, iPrimer) both occupy position 118, and there is a one-nucleotide offset between positions 112 and position 118 when aligning the probe (AAA--) and target (TTTT-) resulting in a bulge forming in the target strand between positions 112 and 118, thereby allowing for formation of an invasive cleavage structure and subsequent detection in an INVADER or INVADER PLUS assay. There would be a two-nucleotide offset if a 117D tcdC allele were present instead, which would destabilize the tertiary structure needed thereby reducing or eliminating the formation of an invasive cleavage structure and subsequent detection in an INVADER or INVADER PLUS assay, thus resulting in allele-specific detection. Thus, in some embodiments, an invasive oligomer and primary probe for detecting a 117del tcdC allele are provided that hybridize to sites that induce a bulge in the target sequence under the primary probe in a complex with a 117del allele, and substantially destabilize the invasive cleavage structure in a complex with a 117D allele. Notably, this approach should render the identity of the nucleotide at position 117 of a 117D allele essentially irrelevant because its effect on spacing rather than its base-pairing properties is what feeds into the assay outcome, thus reducing or eliminating possible errors that the various known non-hypervirulent polymorphisms at this position might otherwise cause.

SEQ ID NO:

| | |
|---|---|
| 106 | 3′-AACGAGATGACCGTAAAUAAA--C-5′ |
| 88 | 3′-CCACACAAAAAACCGTTA...-5′ |
| 3 | 5′-...agggtattgctctactggcatttatttt-ggcgt |
| | * |
| | gttttttggcaat...-3′ |

Shown above are positions 1-22 of SEQ ID NO: 106, positions 11-29 of SEQ ID NO: 88, and positions 89-134 of SEQ ID NO: 3.

The 184T primary probe and invasive oligomer (invader or iPrimer) can be configured so that the primary probe target-hybridizing sequence (e.g., SEQ ID NO: 197-200 or 206-212) and the invasive oligomer hybridize to overlapping sites including position 184, e.g., such that the 5′-nucleotide of the primary probe (whose position corresponds to the 3′-terminal nucleotide of the invasive oligomer) can form a Watson-Crick base pair with the base at position 184 in a complex with a 184T allele, but not in a complex with a 184C allele. The absence of a Watson-Crick base pair at this position when the allele is 184C is intended to reduce or eliminate formation of a recognizable invasive cleavage structure that is formed when a 184T allele is present, and thus provide allele-specific detection. Shown below as examples are SEQ ID NOs: 198 (primary probe target-hybridizing sequence) and 194 (invader) (in part), along with an excerpt of the reverse complement of SEQ ID NO: 5, with an asterisk to mark position 184. Thus, in some embodiments, an invasive oligomer and primary probe for detecting a 184T tcdC allele are provided that hybridize to sites that overlap, wherein the nucleotide of the primary probe at the position corresponding to the 3′-terminal nucleotide of the invasive oligomer forms a Watson-Crick base pair in a complex with a 184T allele but not a 184C allele. In some embodiments, an invasive oligomer and primary probe for detecting a 184T tcdC allele are provided that hybridize to sites that overlap, wherein the 3′-penultimate nucleotide of the invasive oligomer forms a Watson-Crick base pair in a complex with a 183T allele but not a 183C allele.

| SEQ ID NO: | |
|---|---|
| 198 | 5′-TAAACATCAGTTATAGATTCTC-3′ |
| 194 | 5′-...AGGAGGTCATTTCTAATa-3′ |
| 5(rc) | 3′-...TCCTCCAGTAAAGATTAATTT |
| | * |
| | GTAGTCAATATCTAAGAGTTTTTTG...-5′ |

Shown above are positions 1-22 of SEQ ID NO: 198, positions 12-30 of SEQ ID NO: 194, and the reverse complement of positions 167-212 of SEQ ID NO: 5.

Table B. Exemplary secondary detection oligomer pairings with primary detection oligomer 5′-flap sequences. Secondary detection oligomers are referred to by their SEQ ID NO (see the Sequence Table below). In some embodiments, a combination of a secondary detection oligomer in Table B with a compatible primary detection oligomer (primary probe) further comprises an invasive oligomer associated with the compatible primary detection oligomer in Table A.

| Primary Detection Oligomer | Secondary Detection Oligomer |
|---|---|
| 5′ flap sequence | (e.g., FRET cassette) |
| aggccacggacg (e.g., 5′-flap sequence from | 10 |
| SEQ ID NO: 37-39 or 53-54) | |
| cgcgccgagg (e.g., 5′-flap sequence from | 11 |
| SEQ ID NO: 138-167, 201-204, 213-216, | |
| 230-232, or 299) | |
| acggacgcggag (e.g., 5′-flap sequence from | 12 |
| SEQ ID NO: 69, 188, 190, 218-219, | |
| 240-242, or 300-316) | |

In some embodiments, an oligomer is provided that comprises a label and/or is non-extendable. Such an oligomer can be used as a probe or as part of a probe system (e.g., as a FRET cassette in combination with a target-binding detection oligomer). In some embodiments, the labeled oligomer comprises a sequence corresponding to a SEQ ID NO listed in the Detection Oligomer column of Table A, or a target-hybridizing sequence thereof. In some embodiments, the label is a non-nucleotide label. Suitable labels include compounds that emit a detectable light signal, e.g., fluorophores or luminescent (e.g., chemiluminescent) compounds that can be detected in a homogeneous mixture. More than one label, and more than one type of label, can be present on a particular probe, or detection can rely on using a mixture of probes in which each probe is labeled with a compound that produces a detectable signal (see. e.g., U.S. Pat. Nos. 6,180,340 and 6,350,579). Labels can be attached to a probe by various means including covalent linkages, chelation, and ionic interactions. In some embodiments the label is covalently attached. For example, in some embodiments, a detection probe has an attached chemiluminescent label such as, e.g., an acridinium ester (AE) compound (see. e.g., U.S. Pat. Nos. 5,185,439; 5,639,604; 5,585,481; and 5,656,744). A label, such as a fluorescent or chemiluminescent label, can be attached to the probe by a non-nucleotide linker (see. e.g., U.S. Pat. Nos. 5,585,481; 5,656,744; and 5,639,604). In some embodiments, an oligomer is provided that is non-extendible and hybridizes to a site in a C. difficile nucleic acid that overlaps the hybridization site of an additional oligomer in a kit or composition, such as an amplification oligomer (e.g., iPrimer) or an invasive oligomer that is not used as a primer. Hybridization of such oligomers can form a substrate for a structure-specific nuclease, e.g., as part of the detection mechanism in an INVADER or INVADER PLUS assay.

In some embodiments, a labeled oligomer (e.g., comprising a fluorescent label) further comprises a second label that interacts with the first label. For example, the second label can be a quencher. Such probes can be used (e.g., in TaqMan™ assays) where hybridization of the probe to a target or amplicon followed by nucleolysis by a polymerase comprising 5′-3′ exonuclease activity results in liberation of the fluorescent label and thereby increased fluorescence, or fluorescence independent of the interaction with the second label. Such probes can also be used (e.g., in INVADER or INVADER PLUS assays (e.g., as FRET cassettes)). In some embodiments, the labeled oligomer has a SEQ ID NO listed in the Secondary Detection Oligomer column of Table B.

In some applications, one or more probes exhibiting at least some degree of self-complementarity are used to facilitate detection of probe:target duplexes in a test sample without first requiring the removal of unhybridized probe prior to detection. Specific embodiments of such detection probes include, for example, probes that form conformations held by intramolecular hybridization, such as conformations generally referred to as hairpins. Suitable hairpin probes include a “molecular torch” (see. e.g., U.S. Pat. Nos. 6,849,412; 6,835,542; 6,534,274; and 6,361,945) and a “molecular beacon” (see. e.g., U.S. Pat. Nos. 5,118,801 and 5,312,728). Molecular torches include distinct regions of self-complementarity (coined “the target binding domain” and “the target closing domain”) which are connected by a joining region (e.g., a —(CH₂CH₂O)₃-linker) and which hybridize to one another under predetermined hybridization assay conditions. When exposed to an appropriate target or denaturing conditions, the two complementary regions (which can be fully or partially complementary) of the molecular torch melt, leaving the target binding domain available for hybridization to a target sequence when the predetermined hybridization assay conditions are restored. Molecular torches are designed so that the target binding domain favors hybridization to the target sequence over the target closing domain. The target binding domain and the target closing domain of a molecular torch include interacting labels (e.g., fluorescent/quencher) positioned so that a different signal is produced when the molecular torch is self-hybridized as opposed to when the molecular torch is hybridized to a target nucleic acid, thereby permitting detection of probe:target duplexes in a test sample in the presence of unhybridized probe having a viable label associated therewith.

Examples of interacting donor/acceptor label pairs that can be used in connection with the disclosure include fluorescein/tetramethylrhodamine, IAEDANS/fluororescein, EDANS/DABCYL, coumarin/DABCYL, fluorescein/fluorescein, BODIPY® FL/BODIPY® FL, fluorescein/DABCYL, lucifer yellow/DABCYL, BODIPY®/DABCYL, eosine/DABCYL, erythrosine/DABCYL, tetramethylrhodamine/DABCYL, Texas Red/DABCYL, CY5/BHQ1®, CY5/BHQ2®, CY5/BHQ3®, CY5.5/BHQ3®, Quasar 670®/BHQ3®, Quasar 705®/BHQ3®, CY3/BHQ1®, CY3/BHQ2® and fluorescein/QSY7® dye. Those having an ordinary level of skill in the art will understand that when donor and acceptor dyes are different, energy transfer can be detected by the appearance of sensitized fluorescence of the acceptor or by quenching of donor fluorescence. Non-fluorescent acceptors such as DABCYL and the QSY7® dyes advantageously eliminate the potential problem of background fluorescence resulting from direct (i.e., non-sensitized) acceptor excitation. Exemplary fluorophore moieties that can be used as one member of a donor-acceptor pair include fluorescein, HEX, ROX, and the CY dyes (such as CY5). Exemplary quencher moieties that can be used as another member of a donor-acceptor pair include DABCYL, BLACKBERRY QUENCHER® which are available from Berry and Associates (Dexter, MI), and the BLACK HOLE QUENCHER® moieties which are available from Biosearch Technologies, Inc., (Novato, Calif.). One of ordinary skill in the art will be able to use appropriate pairings of donor and acceptor labels for use in various detection formats (e.g., FRET, TaqMan™, INVADER, etc).

In some embodiments, a detection oligomer (e.g., probe, primary probe, or labeled probe) is non-extendable. For example, the labeled oligomer can be rendered non-extendable by a 3′-adduct (e.g., 3′-phosphorylation or 3′-alkanediol), having a 3′-terminal 3′-deoxynucleotide (e.g., a terminal 2′,3′-dideoxynucleotide), having a 3′-terminal inverted nucleotide (e.g., in which the last nucleotide is inverted such that it is joined to the penultimate nucleotide by a 3′ to 3′ phosphodiester linkage or analog thereof, such as a phosphorothioate), or having an attached fluorophore, quencher, or other label that interferes with extension (possibly but not necessarily attached via the 3′ position of the terminal nucleotide). In some embodiments, the 3′-terminal nucleotide is not methylated. In some embodiments, a detection oligomer comprises a 3′-terminal adduct such as a 3′-alkanediol (e.g., hexanediol).

In some embodiments, an oligomer such as a detection oligomer is configured to specifically hybridize to an amplicon generated from C. difficile tcdA, tcdB, tcdC, cdtA, or cdtB (e.g., the oligomer comprises or consists of a target-hybridizing sequence sufficiently complementary to the amplicon for specific hybridization). The target-hybridizing sequence can include additional nucleotides beyond the sequence of any SEQ ID NO or variant thereof present in the oligomer.

Also provided by the disclosure is a reaction mixture for determining the presence or absence of a C. difficile target nucleic acid in a sample. A reaction mixture in accordance with the present disclosure comprises at least one or more of the following: an oligomer combination as described herein for amplification of at least one of C. difficile tcdA, tcdB, tcdC, cdtA, or cdtB or any of the combinations thereof noted above; and at least one detection probe oligomer as described herein for determining the presence or absence of at least one of C. difficile tcdA, tcdB, tcdC, cdtA, or cdtB or any of the combinations thereof noted above. The reaction mixture can further include a number of optional components such as, for example, capture probes (e.g., poly-(k) capture probes as described in US 2013/0209992, which is incorporated herein by reference). For an amplification reaction mixture, the reaction mixture will typically include other reagents suitable for performing in vitro amplification such as, buffers, salt solutions, appropriate nucleotide triphosphates (e.g., dATP, dCTP, dGTP, and one or both of dTTP or dUTP; and/or ATP, CTP, GTP and UTP), and/or enzymes (e.g., a thermostable DNA polymerase, and/or reverse transcriptase and/or RNA polymerase and/or FEN enzyme), and will typically include test sample components, in which a C. difficile nucleic acid may or may not be present. A reaction mixture can include amplification oligomers for at least one of C. difficile tcdA, tcdB, tcdC, cdtA, or cdtB or any of the combinations thereof noted above. In addition, for a reaction mixture that includes a detection probe together with an amplification oligomer combination, selection of amplification oligomers and detection probe oligomers for a reaction mixture are linked by a common target region (i.e., the reaction mixture will include a probe that binds to a sequence amplifiable by an amplification oligomer combination of the reaction mixture).

Also provided by the subject disclosure are kits for practicing the methods as described herein. A kit in accordance with the present disclosure comprises at least one or more of the following: an amplification oligomer combination as described herein for amplification of at least one of C. difficile tcdA, tcdB, tcdC, cdtA, or cdtB or any of the combinations thereof noted above; and at least one detection probe oligomer as described herein for determining the presence or absence of at least one of C. difficile tcdA, tcdB, tcdC, cdtA, or cdtB or any of the combinations thereof noted above. In some embodiments, any oligomer combination described herein is present in the kit. The kits can further include a number of optional components such as, for example, capture probes (e.g., poly-(k) capture probes as described in US 2013/0209992).

Other reagents that can be present in the kits include reagents suitable for performing in vitro amplification such as, e.g., buffers, salt solutions, appropriate nucleotide triphosphates (e.g., dATP, dCTP, dGTP, and one or both of dTTP or dUTP; and/or ATP, CTP, GTP and UTP), and/or enzymes (e.g., a thermostable DNA polymerase, and/or reverse transcriptase and/or RNA polymerase and/or FEN enzyme), and will typically include test sample components, in which a C. difficile nucleic acid may or may not be present. In addition, for a kit that includes a detection probe together with an amplification oligomer combination, selection of amplification oligomers and detection probe oligomers for a reaction mixture are linked by a common target region (i.e., the reaction mixture will include a probe that binds to a sequence amplifiable by an amplification oligomer combination of the reaction mixture). In certain embodiments, the kit further includes a set of instructions for practicing methods in accordance with the present disclosure, where the instructions can be associated with a package insert and/or the packaging of the kit or the components thereof.

Any method disclosed herein is also to be understood as a disclosure of corresponding uses of materials involved in the method directed to the purpose of the method. Any of the oligomers comprising C. difficile nucleic acid sequence and any combinations (e.g., kits and compositions, including but not limited to reaction mixtures) comprising such an oligomer are to be understood as also disclosed for use in detecting or quantifying toxigenic C. difficile, and for use in the preparation of a composition for detecting toxigenic C. difficile.

Broadly speaking, methods can comprise one or more of the following components: target capture, in which C. difficile nucleic acid (e.g., from a sample, such as a clinical sample) is annealed to a capture oligomer (e.g., a specific or nonspecific capture oligomer); isolation (e.g., washing, to remove material not associated with a capture oligomer); amplification; and amplicon detection, which for example can be performed in real time with amplification. Certain embodiments involve each of the foregoing steps. Certain embodiments involve exponential amplification, optionally with a preceding linear amplification step. Certain embodiments involve exponential amplification and amplicon detection. Certain embodiments involve any two of the components listed above. Certain embodiments involve any two components listed adjacently above (e.g., washing and amplification, or amplification and detection).

In some embodiments, amplification comprises (1) contacting the sample with at least two oligomers for amplifying at least one of C. difficile tcdA, tcdB, tcdC, cdtA, or cdtB or any of the combinations thereof noted above, where the oligomers include at least two amplification oligomers as described above (e.g., one or more oriented in the sense direction and one or more oriented in the antisense direction for exponential amplification); (2) performing an in vitro nucleic acid amplification reaction, where any of at least one of C. difficile tcdA, tcdB, tcdC, cdtA, or cdtB or any of the combinations thereof noted above present in the sample is used as a template for generating an amplification product; and (3) detecting the presence or absence of the amplification product, thereby determining the presence or absence of at least one of C. difficile tcdA, tcdB, tcdC, cdtA, or cdtB or any of the combinations thereof noted above in the sample.

A detection method in accordance with the present disclosure can further include the step of obtaining the sample to be subjected to subsequent steps of the method. In certain embodiments, “obtaining” a sample to be used includes, for example, receiving the sample at a testing facility or other location where one or more steps of the method are performed, receiving the sample from a subject or person providing or assisting in treatment of a subject, and/or retrieving the sample from a location (e.g., from storage or other depository) within a facility where one or more steps of the method are performed.

In certain embodiments, the method includes purifying C. difficile nucleic acid from other components (e.g., non-nucleic acid components) in a sample before an amplification (e.g., a capture step). Such purification can include methods of separating and/or concentrating organisms contained in a sample from other sample components, or removing or degrading non-nucleic acid sample components (e.g., protein, carbohydrate, salt, lipid, etc). In some embodiments, a sample such as a crude sample is contacted with a wash liquid (e.g., water, buffer, detergent solution, etc.), for example, by associating the sample with a swab and transferring the swab into the wash liquid, wherein at least some impurities are released into the wash liquid. Then at least partially purified sample can be released from the swab into a carrier liquid (e.g., water, buffer, a lysis solution, etc.). In some embodiments, RNA in the sample is degraded (e.g., with RNase and/or heat), and optionally the RNase is removed or inactivated and/or degraded RNA is removed.

In particular embodiments, purifying the nucleic acid includes capturing the nucleic acid to specifically or non-specifically separate the nucleic acid from other sample components. Non-specific target capture methods can involve selective precipitation of nucleic acids from a substantially aqueous mixture, adherence of nucleic acids to a support that is washed to remove other non-nucleic acid sample components, or other approaches for physically separating nucleic acids from a mixture that contains or is suspected of containing C. difficile nucleic acid and other sample components.

Target capture can occur in a solution phase mixture that contains one or more capture probe oligomers that hybridize to the C. difficile target sequence under hybridizing conditions. For embodiments wherein the capture probe oligomers comprise a capture probe tail, the C. difficile-target:capture-probe complex is captured by applying hybridization conditions so that the capture probe tail hybridizes to an immobilized probe, e.g., associated with a support. Certain embodiments use a particulate solid support, such as paramagnetic beads.

Isolation can follow capture, wherein the complex on the solid support is separated from other sample components. Isolation can be accomplished by any appropriate technique (e.g., washing a support associated with the C. difficile-sequence one or more times (e.g., 2 or 3 times) to remove other sample components and/or unbound oligomer). In embodiments using a particulate solid support, such as paramagnetic beads, particles associated with the C. difficile-target can be suspended in a washing solution and retrieved from the washing solution, in some embodiments by using magnetic attraction. To limit the number of handling steps, the C. difficile target nucleic acid can be amplified by simply mixing the C. difficile target sequence in the complex on the support with amplification oligomers and proceeding with amplification steps.

Exponentially amplifying a C. difficile sequence utilizes an in vitro amplification reaction using at least two amplification oligomers that flank a target region to be amplified. In some embodiments, at least two amplification oligomers as described above are provided. In some embodiments, a plurality of pairs of amplification oligomers is provided, wherein the plurality comprises oligomer pairs configured to hybridize to at least 2, 3, or 4 of tcdA, tcdB, tcdC, cdtA, and cdtB, or at least 2, 3, or 4 of tcdA, tcdB, tcdC, and cdtB. The amplification reaction can be cycled or isothermal. Suitable amplification methods include, for example, replicase-mediated amplification, polymerase chain reaction (PCR), ligase chain reaction (LCR), strand-displacement amplification (SDA), and transcription-mediated amplification (TMA).

A detection step can be performed using any of a variety of known techniques to detect a signal specifically associated with the amplified target sequence, such as by hybridizing the amplification product with a labeled detection probe and detecting a signal resulting from the labeled probe (including from label released from the probe following hybridization in some embodiments). In some embodiments, the labeled probe comprises a second moiety, such as a quencher or other moiety that interacts with the first label, as discussed above. The detection step can also provide additional information on the amplified sequence, such as all or a portion of its nucleic acid sequence. Detection can be performed after the amplification reaction is completed, or can be performed simultaneously with amplifying the target region (e.g., in real time). In one embodiment, the detection step allows homogeneous detection (e.g., detection of the hybridized probe without removal of unhybridized probe from the mixture (see. e.g., U.S. Pat. Nos. 5,639,604 and 5,283,174)). In some embodiments, the nucleic acids are associated with a surface that results in a physical change, such as a detectable electrical change. Amplified nucleic acids can be detected by concentrating them in or on a matrix and detecting the nucleic acids or dyes associated with them (e.g., an intercalating agent such as ethidium bromide or cyber green), or detecting an increase in dye associated with nucleic acid in solution phase. Other methods of detection can use nucleic acid detection probes that are configured to specifically hybridize to a sequence in the amplified product and detecting the presence of the probe:product complex, or by using a complex of probes that can amplify the detectable signal associated with the amplified products (e.g., U.S. Pat. Nos. 5,424,413; 5,451,503; and 5,849,481; each incorporated by reference herein). Directly or indirectly labeled probes that specifically associate with the amplified product provide a detectable signal that indicates the presence of the target nucleic acid in the sample. In particular, the amplified product will contain a target sequence in or complementary to a sequence in the C. difficile chromosome, and a probe will bind directly or indirectly to a sequence contained in the amplified product to indicate the presence of C. difficile nucleic acid in the tested sample.

In some embodiments, amplified product is detected through an invasive cleavage assay that provides means for forming an invasive cleavage structure that requires the presence of a target nucleic acid. The assay further involves cleaving the invasive cleavage structure to release distinctive cleavage products. A cleavage agent such as a FEN enzyme, for example, is used to cleave the target-dependent invasive cleavage structure, thereby resulting in cleavage products that indicate the presence of specific target nucleic acid sequences in the sample. When two oligonucleotides hybridize to a target nucleic acid strand such that they form an overlapping invasive cleavage structure, as defined above, invasive cleavage can occur. Through the interaction of a cleavage agent (e.g., FEN enzyme) and the upstream oligonucleotide (i.e., the invasive probe), the cleavage agent can be made to cleave the downstream oligonucleotide (i.e., the primary probe) at an internal site such that a distinctive fragment is produced. The fragment, sometimes referred to as a “liberated flap” or “cleaved 5′ flap” or simply a “flap” can then itself interact with a secondary probe such as a FRET cassette (e.g., by participating as an invasive probe in a subsequent reaction that generates a detectable signal (e.g., a fluorescent signal)). Such embodiments are described in U.S. Pat. Nos. 5,846,717, 5,985,557, 5,994,069, 6,001,567, and 6,090,543, WO 97/27214, WO 98/42873, Nat. Biotech., 17:292 (1999), PNAS, 97:8272 (2000), and WO 2016/179093. More specifically, a plurality of INVADER reactions, e.g., combined in a single reaction mixture, can be used for the multiplex applications disclosed herein, including detection of two or more of tcdA, tcdB, tcdC, cdtA, and cdtB, or two or more of of tcdA, tcdB, tcdC, and cdtB. Additionally, an internal control can also be included in the multiplex amplification and detection procedure.

Invasive cleavage assays can be used for detecting specific target sequences in unamplified, as well as amplified DNA (e.g., PCR product(s)), including genomic DNA, RNA, or an amplicon thereof. The primary probe and the invasive probe hybridize in tandem to the target nucleic acid to form an overlapping structure. An unpaired “flap” is included on the 5′ end of the primary probe. A cleavage agent (e.g. a FEN enzyme, such as the Cleavase® enzymes available from Hologic, Inc.) recognizes the overlap and cleaves off the unpaired 5′ flap. In some embodiments, in a secondary reaction, this cleaved product serves as an invasive probe on a FRET cassette to again create a structure recognized by the structure-specific enzyme. When the two labels on a single FRET cassette are separated by cleavage, a detectable fluorescent signal above background fluorescence is produced. Consequently, cleavage of this second invasive cleavage structure results in an increase in fluorescence, indicating the presence of the target sequence.

Alternatively, in embodiments that detect the amplified product near or at the end of the amplification step, a linear detection probe can be used to provide a signal to indicate hybridization of the probe to the amplified product. One example of such detection uses a luminescentally labeled probe that hybridizes to target nucleic acid. The luminescent label is then hydrolyzed from non-hybridized probe. Detection is performed by chemiluminescence using a luminometer (see, e.g., International Patent Application Pub. No. WO 89/002476). In other embodiments that use real-time detection, the detection probe can be a hairpin probe such as a molecular beacon, molecular torch, or hybridization switch probe that is labeled with a reporter moiety that is detected when the probe binds to amplified product. Such probes can comprise target-hybridizing sequences and non-target-hybridizing sequences. Various forms of such probes are described, e.g., in U.S. Pat. Nos. 5,118,801; 5,312,728; 5,925,517; 6,150,097; 6,849,412; 6,835,542; 6,534,274; and 6,361,945; and US Patent Application Pub. Nos. 2006/0068417A1 and 2006/0194240A1).

In some embodiments, one or more of an internal amplification control polynucleotide (“internal control”; e.g., a plasmid, plasmid fragment, or other polynucleotide, generally with a sequence unrelated to C. difficile sequences, e.g., to tcdA, tcdB, tcdC, cdtA, and cdtB), and oligomers for amplifying and detecting the internal control are provided as components of a composition or kit disclosed herein and/or are used in a method disclosed herein. Detection of an amplicon from the internal control can serve to avoid false negatives due to instrument or reagent failures when no target sequences are detected.

Below is a table illustrating interpretation of various possible results obtained from methods according to this disclosure. In some embodiments, results are interpreted according to this table. U indicates that detection of an amplicon is expected but unnecessary to the call. In embodiments where reagents for amplifying and/or detecting one or more targets shown below are not used, such as cdtA or tcdA, the table can be applied as if the relevant column referred to the particular target being detected (e.g., cdtB or tcdB).

TABLE C
Interpretation of Results

| Amplicon Detection Results | | | | Toxigenic | Hyper- |
|---|---|---|---|---|---|
| tcdA or | tcdC 117 del | | Internal | C. dfficile | virulence |
| tcdB | or tcdC 184T | cdtA or cdtB | control | Call | Call |
| + | − | − | U | Positive | Negative |
| + | − | + | U | Positive | Negative |
| + | + | + | U | Positive | Positive |
| − | − | + | U | Positive | Negative |
| − | + | − | U | Negative | Negative |
| − | + | + | U | Positive | Negative |
| − | − | − | + | Negative | Negative |
| − | − | − | − | Invalid | Invalid |

Abbreviations are as defined above.

### EXAMPLES

The following examples are provided to illustrate certain disclosed embodiments and are not to be construed as limiting the scope of this disclosure in any way.

All references to SEQ ID NOs in the Examples section include the cytosine methylation, labels, and other features indicated in the Table of Sequences below.

General Reagents and Methods. Unless otherwise indicated, non-specific target capture was used to isolate nucleic acid. Exemplary non-specific target capture reagents and procedures are disclosed in Becker et al., US2013/0209992A1. Target capture procedures were generally performed using a Panther or Panther Fusion™ instrument.

Unless otherwise indicated, amplifications were performed on a Panther Fusion™ instrument or an ABI 7500 Fast™ instrument. Unless otherwise indicated, amplifications were performed with combinations of oligomers disclosed above designed to detect sequences amplified from one or more of tcdA, tcdB, tcdC, cdtA, and cdtB. In some experiments, a set of primary probes for tcdC was used wherein a first primary probe targeted a site comprising position 117 of SEQ ID NO: 3 (to detect the 117del allele of tcdC) and a second primary probe targeted a site comprising position 184 of SEQ ID NO: 5 (to detect the 184T allele of tcdC). These sequences were detected with at least forward and reverse amplification oligomers and at least one primary detection oligomer configured to specifically hybridize to the target sequence(s) in INVADER PLUS assays as described above. In some reactions, one or more amplification oligomers or an extension product thereof served as an invasive probe, i.e., an iPrimer. In some reactions, a separate invasive probe was used. An internal control plasmid was also generally included in the reactions.

Cleavase used in these Examples was the Afu FEN-1 endonuclease described in U.S. Pat. No. 9,096,893.

Amplification reagents were dNTPs at 25 M each, a commercially available hot-start Taq polymerase, MgCl₂, Cleavase, MOPS and Tris buffers, non-acetylated BSA, and other components supplied by Promega® Master Mix. Primers were supplied at a final concentration of 0.2-0.75 μM unless otherwise indicated. Oligomer annealing temperatures were from 65° C. to 69° C. and a standard extension temperature of 72° C. was used for 10 cycles, followed by a series of cycles having a 65° C. INVADER reaction step. Unless otherwise indicated, data was collected for 35 INVADER reaction cycles. “No detection” indicates that Ct had not occurred by the 35^{th} cycle of collected data.

As noted above, detection of amplicons used INVADER PLUS chemistry. One or more FRET cassettes were included that collectively corresponded to the primary probes. Where two tcdC primary probes were used, one FRET cassette (e.g., SEQ ID NO: 11) was used that would interact with fragments generated from either tcdC primary probe. Where tcdA and/or tcdB primary probes were used, one FRET cassette (e.g., SEQ ID NO: 12) was used that would interact with fragments (liberated flaps) generated from either of the tcdA and tcdB primary probes. Where at least one cdtB primary probe was used, a FRET cassette (e.g., SEQ ID NO: 10) was used that would interact with fragments (liberated flaps) generated therefrom. The FRET cassettes were labeled with a interactive label pair in an energy transfer relationship, where fluorescence emission was quenched when both members of the label pair were attached to the FRET cassette. Thus, a positive signal for a given target was generally interpreted as indicating that a target sequence was present and amplified by a corresponding set of primers; that an invasive cleavage structure comprising a primer or detection oligomer (invader oligomer), the amplicon, and the corresponding primary probe had been formed and cleaved by Cleavase; that the flap thereby liberated from the primary probe formed an invasive cleavage structure with the corresponding FRET cassette, which was then cleaved by Cleavase, thereby allowing fluorescent detection of a labeled cleavage product of the FRET cassette.

### Example 1. Detection of tcdC Alleles

The tcdC amplification and detection oligomers shown in the Table of Sequences were designed. Combinations of oligomers were tested with samples containing 0 (negative control), 5, 10, or 100 copies of C. difficile tcdC in 25 μL reactions. Reactions were generally run in triplicate.

TABLE D
tcdC amplification oligomer sets

| Set | Oligomers (SEQ ID NOs) |
|---|---|
| 1 | 247 & 249 |
| 2 | 247 & 250 |
| 3 | 247 & 255 |
| 4 | 247 & 251 |
| 5 | 247 & 252 |
| 6 | 247 & 258 |

Set 1 gave signal that was near but not at the threshold at 100 copies, with no detection at lower values. Set 2 gave Ct values in the range of 31-35 at 100 copies no detection at 5 or 10 copies. Set 3 gave Ct values in the range of 29-32 at 100 copies and no detection at 5 or 10 copies. Set 4 gave Ct values in the range of 32-35 at 100 copies and no detection at 5 or 10 copies. Set 5 gave Ct values in the range of 31-32 at 100 copies (with detection in 2/3 triplicates) and no detection at 5 or 10 copies (with detection in 2/3 triplicates) and no detection at 5 or 10 copies. Set 6 gave Ct values in the range of 30-35 at 100 copies and no detection at 5 or 10 copies. Unless indicated otherwise, detection occurred in 3/3 triplicates.

The reactions were run in multiplex with tcdA, tcdB, and cdtB primers to confirm that the primer systems did not interfere with each other. Regardless of which tcdC oligomer set was used, detection of tcdA/B was consistent at a Ct in the range of 22-25 for the 100 copy reactions and detection of cdtB was consistent at a Ct in the range of 27-31 for the 100 copy reactions.

Invasive probe and primary detection oligomers targeting the 117del and 184T tcdC alleles (SEQ ID NOs: 86, 161, 165-167, 196, and 204) were tested in reactions with 10 copies of the relevant tcdC allele as follows (5 replicates each).

Cts in this paragraph are for detection of 117del tcdC. With SEQ ID NO: 86 and 161, Cts were about 35 with detection occurring in 2/5 replicates. With SEQ ID NO: 86 and 167, Cts were in the range of 30-35 with detection occurring in 5/5 replicates. With SEQ ID NO: 86 and 165, Cts were in the range of 32-35 with detection occurring in 4/5 replicates. With SEQ ID NO: 86 and 166, Cts were in the range of 30-34 with detection occurring in 4/5 replicates.

An exemplary pair of invasive probe and primary detection oligomers selected from those described above was also tested with 117T and 117A tcdC alleles. At 10, 100, and 1000 copies per reaction, the Ct was delayed by 12 cycles for 1000 copies per reaction with no detection for the 10 or 100 copy concentrations for the 117T allele relative to the 117del allele, indicating that the probe system could discriminate 117del tcdC (associated with hypervirulence) from 117T tcdC (not associated with hypervirulence). Additionally, the 117A tcdC allele (wild-type, not hypervirulent) did not generate positive signal at 10, 100, and 1000 copies per reaction, indicating that the system does not detect the 117A allele.

Detection of 184T tcdC was performed in duplicate unless otherwise noted. The template sequence used in these tests also contained a T at position 183, as in SEQ ID NO: 7, as the 184T polymorphism has been observed to coincide with a 183T polymorphism. Results are shown in the following table, given as ranges within which the observed Cts fell. “Copies” indicates copies per reaction.

TABLE E
Ct results for detection of 184T tcdC

| Invasive | | Ct (N/D = No Detection) | | |
|---|---|---|---|---|
| oligomer | Primary Probe | | | |
| SEQ ID NO | SEQ ID NO | 100 copies | 1000 copies | 10,000 copies |
| 195 | 202 | N/D | N/D | 33-35 |
| 195 | 203 | 26-27 | 23-24 | 20-21 |
| 195 | 204 | N/D | N/D | 34-35 |
| 196 | 202 | 26-27 | 23-24 | 20-21 |
| 196 | 203 | N/D | 34-35 (1 of 2) | 31-33 |
| 196 | 204 | 25.5-26.5 | 22-23 | 19-20 |
| 205 | 215 | 24-25 | 21-22 | 17-18 |
| 205 | 216 | 31-32 | 28-29 | 24-25 |

Comparative reactions were performed with a wild-type, non-hypervirulent tcdC template (183C 184C, as in positions 183-184 of SEQ ID NO: 2) and a 183T 184C non-hypervirulent tcdC template (as in positions 183-184 of SEQ ID NO: 1) in duplicate at 10,000 copies per reaction. Results are shown below.

TABLE F
Ct results for detection of 183T 184C and 183C 184C tcdC

| Invasive | | | |
|---|---|---|---|
| oligomer | Primary Probe | tcdC allele (N/D = No Detection) | |
| SEQ ID NO | SEQ ID NO | 183T 184C | 183C 184C |
| 195 | 202 | N/D | N/D |
| 195 | 203 | 23-24 | N/D |
| 195 | 204 | N/D | N/D |
| 196 | 202 | N/D | N/D |
| 196 | 203 | N/D | N/D |
| 196 | 204 | N/D | N/D |
| 205 | 215 | 32-34 | N/D |
| 205 | 216 | 23-24 | N/D |

### Example 2. Detection of tcdB

Testing of forward amplification oligomers SEQ ID NOs: 266 and 269, reverse iPrimers SEQ ID NOs: 317 and 324, and a primary probe oligomer of SEQ ID NO: 312 gave a sensitivity (Ct) in the range of 26-28 for 10 copies per reaction and was compatible in multiplex with tcdC amplification and detection oligomers. Cross-reactivity was observed with template from another Clostridium species, C. soderllii (strain ATCC9714 or JG6382).

Testing of an oligomer set including forward amplification oligomer SEQ ID NOs: 339, a reverse iPrimer selected from SEQ ID NOs: 323 and 327, and a primary probe oligomer selected from SEQ ID NO: 307 and 300 gave a sensitivity (Ct) of generally below 30 at 10 copies per reaction) but generated some signal in negative control reactions.

Two additional oligomer sets were tested, and results are shown below. tcdB oligomer set 1 included SEQ ID NOs: 274, 277, 305, 306, and 336; tcdB oligomer set 2 included SEQ ID NOs: 274, 275, 301, 305, and 336. Lysates from two different C. difficile strains (1: ATCC BAA-1805; and 2: CCUG20309) which had differing tcdB sequences were tested in duplicate using the indicated number of colony forming units (CFU) as input material.

TABLE G
Ct results for detection of tcdB in different strains

| | | tcdB oligomer set (N/D = No Detection) | |
|---|---|---|---|
| Strain | CFU/reaction | 1 | 2 |
| 1 | 100 | 26-27 | 25-26 |
| 1 | 10 | 29-30 | 28-29 |
| 1 | 1 | 32-34 | 30-31* |
| 2 | 1000 | 23-24 | 23-24 |
| 2 | 100 | 25-26 | 27-28 |
| 2 | 10 | 29-30 | N/D |
| 2 | 1 | 33-35* | N/D |

*detection in 1/2 duplicates

tcdB oligomer sets 1 and 2 did not generate significant signal in negative control reactions, and were compatible in multiplex with tcdC amplification oligomers. No cross reactivity with C. soderlii was expected based on the number of mismatches between these oligomer sets and the C. soderlii tcsL sequence, which is the C. soderlii sequence most similar to C. difficile tcdB.

### Example 3. Detection of cdtB

C. difficile can contain a binary toxin locus with the cdtA and cdtB binary toxin genes. Oligomer designs for detection of either binary toxin gene. It was found that cdtB designs could provide better specificity and performance in multiplex reactions. Some cross-reactivity was observed in initial experiments but subsequent testing with newly prepared synthetic target DNA essentially eliminated the cross-reactivity, suggesting that contamination of the original target DNA stock may have been responsible.

Representative cdtB oligomer sets, listed in the order of forward amplification oligomer, primary probe, and reverse iPrimer, are SEQ ID NO: 33, 37, and 40; or 31, 38, and 41; or permutations thereof, e.g., in which SEQ ID NO: 33 is exchanged for SEQ ID NO: 31 or vice versa.

Results from a representative cdtB oligomer set were as shown below. In these experiments, data for 38 cycles of INVADER reaction were collected.

TABLE H
Ct results for detection of cdtB

| CFU/reaction | Ct |
|---|---|
| 1000 | 26-27 |
| 100 | 29-30 |
| 10 | 33-35 |
| 1 | 36-38 |

### Example 4. Detection of tcdA

To avoid loss of sensitivity in the event of mutations that affect detection of tcdB (the Toxin B gene of the C. difficile pathogenic locus), oligomers for detection of tcdA were designed that can be multiplexed with oligomers for detecting tcdB and the other genes discussed above.

Exemplary tcdA oligomer sets contained SEQ ID NO: 64 or 65 as a forward iPrimer, SEQ ID NO: 68 or 69 as a primary probe, and any one of SEQ ID NO: 70-73 as a reverse primer. In the results shown below (Table I), tcdA set 1 used SEQ ID NO: 64, 69, and 72; and tcdA set 2 used SEQ ID NO: 65, 69, and 72. Data were collected for 38 cycles of INVADER reaction. These results were performed in multiplex with tcdB, tcdC, and cdtB oligomer sets with 6, 12, or 25 copies of target DNA per reaction (n=8 for each condition); as noted above, a FRET cassette was used that detected cleavage of both tcdA and tcdB primary probes. The combined tcdA/tcdB detection performed well with both tcdA systems present. Additionally, the tcdA oligomers of set 1 were tested in the absence of tcdB oligomers with 100 copies of tcdA+ target per reaction (n=6, including 2 each of wild type, 184T, and 117del tcdC genotypes), and the results were positive with Cts of 27-28 and consistent with there being no negative impact from using the tcdA oligomers in multiplex. Target DNA was from C. difficile ATCC4118, which carries a 117del tcdC allele, at the indicated number of copies per reaction. Positivity indicates the proportion of replicates in which positive signal was observed. Regardless of which set was used, at least one of tcdA/B, tcdC, and cdtB were detected in every replicate. Negative controls were as expected (not shown).

TABLE I
Ct results for detection of tcdA/ B, tcdC, and cdtB in multiplex

| | tcdA oligomer set | | | |
|---|---|---|---|---|
| Copies/ | 1 | | 2 | |
| reaction | tcdA/B Ct | Positivity | tcdA/B Ct | Positivity |
| 6 | 21-26 | 7/8 | 22-28 | 7/8 |
| 12 | 20-22 | 8/8 | 21-24 | 8/8 |
| 25 | 19-22 | 8/8 | 20-23 | 8/8 |
| | tcdC Ct | Positivity | tcdC Ct | Positivity |
| 6 | 28-32 | 7/8 | 28-33 | 5/8 |
| 12 | 27-32 | 7/8 | 28-37 | 7/8 |
| 25 | 26-34 | 8/8 | 26-31 | 8/8 |
| | cdtB Ct | Positivity | cdtB Ct | Positivity |
| 6 | 27-31 | 3/8 | 27-32 | 6/8 |
| 12 | 26-36 | 6/8 | 26-30 | 8/8 |
| 25 | 25-28 | 8/8 | 25-32 | 8/8 |

TABLE OF SEQUENCES

| | | |
|---|---|---|
| SEQ ID NO | Description | Sequence 5′-3′ |

Exemplary C. difficile sequences

| | | |
|---|---|---|
| 1 | tcdC from GenBank | ATGTTTTCTAAAAAAAATGAGGGTAACGAATTTAGTAATGAAGGAAAAGGAAGCTCT |
| | DQ870676.1 | AAGAAAATAATTAAATTCTTTAAGAGCACAAAGGGTATTGCTCTACTGGCATTTATT |
| | (117 = G, 183 = T) | TTGGGCGTGTTTTTGGCAATATATCCTCACCAGCTTGTTCTGAAGACCATGAGGAGG |
| | | TCATTTCTAATCAAACATCAGTTATAGATTCTCAAAAAACAGAAATAGAAACTTTAA |
| | | ATAGCAAATTGTCTGATGCTGAACCATGGTTCAAAATGAAAGACGACGAAAAGAAAG |
| | | CTATTGAAGCTGAAAATCAACGTAAAGCTGAAGAAGCTAAAAAGGCTGAAGAACAAC |
| | | GTAAAAAAGAAGAAGAAGAGAAGAAAGGATATGATACTGGTATTACTTATGACCAAT |
| | | TAGCTAGAACACCTGATGATTATAAGTACAAAAAGGTAAAATTTGAAGGTAAGGTTA |
| | | TTCAAGTTATTGAAGATGGTGATGAGGTGCAAATAAGATTAGCTGTGTCTGGAAATT |
| | | ATGATAAGGTCGTACTATGTAGTTATAAAAAATCAATAACTCCTTCAAGAGTGTTAG |
| | | AGGATGATTACATAACTATAAGAGGTATAAGTGCTGGAACTATAACTTATGAATCAA |
| | | CTATGGGTGGAAATATAACTATACCAGGGATAGCTGTAGAGAAAATAAATTAA |
| 2 | tcdC from GenBank | ATGTTTTCTAAAAAAAATGAGGGTAACGAATTTAGTAATGAAGGAAAAGGAAGCTCT |
| | EU075382.1 (partial, | AAGAAAATAATTAAATTCTTTAAGAGCACAAAGGGTATTGCTCTACTGGCATTTATT |
| | 117 = a, 183 = C) | TTAGGCGTGTTTTTTGGCAATATATCCTCACCATCTTGTTCTGAAGACCATGAGGAG |
| | | GTCATTTCTAACCAAACATCAGTTATAGATTCTCAAAAAACAGAAATAGAAACTTTA |
| | | AATAGCAAATTGTCTGATGCTGAACCATGGTTCAAAATGAAAGACGACGAAAAGAAA |
| | | GCTATTGAAGCTGAAAATCAACGTAAAGCTGAAGAAGCTAAAAAAGCTGAAGAAGCT |
| | | AAAAAGGCTGAAGAACAACGCAAAAAAGAAGAAGAGGAGAAGAAAGGATATGATACT |
| | | GGTATTACTTATGACCAATTAGCTAGAACACCTGATGATTATAAGTACAAAAAGGTA |
| | | AAATTTGAAGGTAAGGTTATTCAA |
| 3 | tcdC 117delA | ATGTTTTCTAAAAAAAATGAGGGTAACGAATTTAGTAATGAAGGAAAAGGAAGCTCT |
| | (with dash at | AAGAAAATAATTAAATTCTTTAAGAGCACAAAGGGTATTGCTCTACTGGCATTTATT |
| | deletion site) | TT-GGCGTGTTTTTTGGCAATATATCCTCACCAGCTTGTTCTGAAGACCATGAGGAG |
| | | GTCATTTCTAATCAAACATCAGTTATAGATTCTCAAAAAACAGAAATAGAAACTTTA |
| | | AATAGCAAATTGTCTGATGCTGAACCATGGTTCAAAATGAAAGACGACGAAAAGAAA |
| | | GCTATTGAAGCTGAAAATCAACGTAAAGCTGAAGAAGCTAAAAAGGCTGAAGAACAA |
| | | CGTAAAAAAGAAGAAGAAGAGAAGAAAGGATATGATACTGGTATTACTTATGACCAA |
| | | TTAGCTAGAACACCTGATGATTATAAGTACAAAAAGGTAAAATTTGAAGGTAAGGTT |
| | | ATTCAAGTTATTGAAGATGGTGATGAGGTGCAAATAAGATTAGCTGTGTCTGGAAAT |
| | | TATGATAAGGTCGTACTATGTAGTTATAAAAAATCAATAACTCCTTCAAGAGTGTTA |
| | | GAGGATGATTACATAACTATAAGAGGTATAAGTGCTGGAACTATAACTTATGAATCA |
| | | ACTATGGGTGGAAATATAACTATACCAGGGATAGCTGTAGAGAAAATAAATTAA |
| 4 | tcdC 117T | ATGTTTTCTAAAAAAAATGAGGGTAACGAATTTAGTAATGAAGGAAAAGGAAGCTAT |
| | | AAGAAAATAATTAAATTCTTTAAGAGCACAAAGGGTATTGCTCTACTGGCATTTATT |
| | | TTTGGCGTGTTTTTTGGCAATATATCCTCACCAGCTTGTTCTGAAGACCATGAGGAG |
| | | GTCATTTCTAATCAAACATCAGTTATAGATTCTCAAAAAACAGAAATAGAAACTTTA |
| | | AATAGCAAATTGTCTGATGCTGAACCATGGTTCAAAATGAAAGACGACGAAAAGAAA |
| | | GCTATTGAAGCTGAAAATCAACGTAAAGCTGAAGAAGCTAAAAAGGCTGAAGAACAA |
| | | CGTAAAAAAGAAGAAGAAGAGAAGAAAGGATATGATACTGGTATTACTTATGACCAA |
| | | TTAGCTAGAACACCTGATGATTATAAGTACAAAAAGGTAAAATTTGAAGGTAAGGTT |
| | | ATTCAAGTTATTGAAGATGGTGATGAGGTGCAAATAAGATTAGCTGTGTCTGGAAAT |
| | | TATGATAAGGTCGTACTATGTAGTTATAAAAAATCAATAACTCCTTCAAGAGTGTTA |
| | | GAGGATGATTACATAACTATAAGAGGTATAAGTGCTGGAACTATAACTTATGAATCA |
| | | ACTATGGGTGGAAATATAACTATACCAGGGATAGCTGTAGAGAAAATAAATTAA |
| 5 | tcdC | ATGTTTTCTAAAAAAAATGAGGGTAACGAATTTAGTAATGAAGGAAAAGGAAGCTCT |
| | 184C > T | AAGAAAATAATTAAATTCTTTAAGAGCACAAAGGGTATTGCTCTACTGGCATTTATT |
| | | TTGGGCGTGTTTTTTGGCAATATATCCTCACCAGCTTGTTCTGAAGACCATGAGGAG |
| | | GTCATTTCTAATTAAACATCAGTTATAGATTCTCAAAAAACAGAAATAGAAACTTTA |
| | | AATAGCAAATTGTCTGATGCTGAACCATGGTTCAAAATGAAAGACGACGAAAAGAAA |
| | | GCTATTGAAGCTGAAAATCAACGTAAAGCTGAAGAAGCTAAAAAGGCTGAAGAACAA |
| | | CGTAAAAAAGAAGAAGAAGAGAAGAAAGGATATGATACTGGTATTACTTATGACCAA |
| | | TTAGCTAGAACACCTGATGATTATAAGTACAAAAAGGTAAAATTTGAAGGTAAGGTT |
| | | ATTCAAGTTATTGAAGATGGTGATGAGGTGCAAATAAGATTAGCTGTGTCTGGAAAT |
| | | TATGATAAGGTCGTACTATGTAGTTATAAAAAATCAATAACTCCTTCAAGAGTGTTA |
| | | GAGGATGATTACATAACTATAAGAGGTATAAGTGCTGGAACTATAACTTATGAATCA |
| | | ACTATGGGTGGAAATATAACTATACCAGGGATAGCTGTAGAGAAAATAAATTAA |
| 6 | cdtB from | ATGAAAGTACAAATGAGGAATAAAAAGGTATTAAGTTTTTTAACACTTACAGCCATA |
| | GenBank | GTTAGTCAAGCATTAGCATATCCTGTATATGCTCAAACTAGTACAAGTAGCCATTCT |
| | HQ639679 | GATAATAAAAAAGAAATTATAAATGAAGACATACTCACAAACAACGGATTAATGGGA |
| | | TATTATTTCACAGACGAACACTTTAAAGATTTAAAATTAATGGCACCCATAAAAGAT |
| | | GGTAATTTAAAATTTGAAGAAAAGAAAGTAGATAAACTTCTAAATAAAGACAAATCA |
| | | AATGTAAAATCTATACGATGGACAGGAAGAATAATTCCTTCTAAGGATGGTGAATAT |
| | | ACATTATCAACTGATAGAGATGATATTTTAATGCAAGTAAATAATGAGAGTACTATA |
| | | TCAAATACACTTAAAGTTAATATGAAAAAGGGTAAAGAATATAAATTTAGAATAGAG |
| | | CTACAAGATAAAAATTTAGGTTCAATAGATAATTTATCATCACCAAATCTTTATTGG |
| | | GAATTAGATGGTATTAAGAAAATTATACCAGCAGAAAATTTATTCTTAAGAGATTAT |
| | | TCTAATATAGAAAAAAATGATCCATTTATCCCAAATAACAATTTCTTTGACCCAAGG |
| | | TTGATGTCTGATTGGGAAGACGAAGATTTGGATACAGATAATGATAATATACCAGAT |
| | | TCATATGAACGAAATGGATATACTATTAAGGACTTAATTGCAGTTAAGTGGGAAGAT |
| | | AGCTTTGCAGAACAAGGCTATAAGAAATATGTATCAAATTATTTAGAGTCAAATACT |
| | | GCTGGAGATCCATATACAGATTATGAAAAAGCTTCAGGTTCTTTTGACAAGGCTATA |
| | | AAGACCGAAGCAAGAGATCCGTTAGTTGCAGCGTATCCAATTGTTGGAGTAGGTATG |
| | | GAAAAATTAATTATATCTACAAATGAACATGCCTCTACTGATCAAGGTAAAACTGTT |
| | | TCCAGAGCTACTACTAACAGTAAAACTGAATCTAATACAGCTGGTGTTTCTGTTAAT |
| | | GTAGGATATCAAAATGGATTCACAGCTAATGTAACTACAAATTATTCCCATACAACA |
| | | GATAATTCAACTGCCGTTCAAGATAGTAATGGAGAATCATGGAATACTGGATTAAGT |
| | | ATAAACAAAGGAGAATCTGCATATATAAATGCAAATGTTAGATATTACAACACAGGT |
| | | ACTGCACCTATGTACAAAGTGACACCAACAACAAATTTAGTGTTAGATGGAGATACA |
| | | TTATCAACTATCAAAGCACAAGAAAATCAAATTGGCAATAATCTATCTCCTGGAGAT |
| | | ACTTATCCCAAAAAAGGGCTTTCACCTCTGGCTCTTAACACAATGGATCAATTTAGC |
| | | TCTAGACTGATTCCTATAAATTATGATCAATTAAAAAAATTAGATGCTGGAAAGCAA |
| | | ATTAAATTAGAAACAACACAAGTAAGTGGAAATTTTGGTACAAAAAATAGTTCTGGA |
| | | CAAATAGTAACAGAAGGAAATAGTTGGTCAGACTATATAAGTCAAATTGACAGTATT |
| | | TCTGCATCTATTATATTAGATACAGAGAATGAATCTTACGAAAGAAGAGTTACTGCT |
| | | AAAAATTTACAGAATCCAGAAGATAAAACACCTGAACTTACAATTGGAGAAGCAATT |
| | | GAAAAAGCTTTTGGCGCTACTAAAAAAGATGGTTTGTTATATTTTAATGATATACCA |
| | | ATAGATGAAAGTTGTGTTGAACTCATATTTGATGATAATACAGCCAATAAGATTAAA |
| | | GATAGTTTAAAAACTTTGTCTGATAAAAAGATATATAATGTTAAACTTGAAAGAGGA |
| | | ATGAATATACTTATAAAAACACCAACTTACTTTACTAATTTTGATGATTACAATAAT |
| | | TACCCTAGTACATGGAGTAATGTCAATACTACGAATCAAGATGGTTTACAAGGCTCA |
| | | GCAAATAAATTAAATGGTGAGACAAAGATTAAAATACCTATGTCTAAGCTAAAACCT |
| | | TATAAACGTTATGTTTTTAGTGGATATTCAAAGGATCCTTTAACATCTAATTCAATA |
| | | ATTGTAAAGATAAAAGCAAAAGAAGAAAAAACGGATTATTTGTTACCAGAACAAGGA |
| | | TATACAAAGTTTAGTTATGAATTTGAAACTACTGAAAAAGATTCTTCTAATATAGAG |
| | | ATAACATTAATTGGTAGTGGTACAACATACTTAGATAACTTATCTATTACAGAACTG |
| | | AATAGTACTCCTGAAATACTTAATGAACCAGAAGTTAAAATTCCAACTGACCAAGAA |
| | | ATAATAGATGCACATAAAATATATTCTGCAGATTTAAATTTTAATCCAAGTACAGGA |
| | | AATGCTTATATAAATGGTATGTATTTTACACCAACACAAACTAATAAAGAAGCTCTC |
| | | GATTATATCCAAAAATATAGAGTTGAAGCTACTTTACAATATTCTGGATTTAAAGAT |
| | | ATTGGAACTAAAGATAAAGAAATGCGTAATTATTTAGGAGATCCAAATCAACCTAAA |
| | | AACTAATTATGTCAATCTTAGGAGTTATTTTACAGGTGGAGAAAATATTATGACATA |
| | | CAAGAAATTAAGAATATAGCAATTACTCCAGATGATAGAGAGTTATTAGTTCTTAGT |
| | | GTTGATTAG |
| 7 | tcdA from GenBank | ATGTCTTTAATATCTAAAGAAGAGTTAATAAAACTCGCATATAGCATTAGACCAAGA |
| | KC292125 | GAAAATGAGTATAAAACTATACTAACTAATTTAGACGAATATAATAAGTTAACTACA |
| | | AACAATAATGAAAATAAATATTTACAATTAAAAAAACTAAATGAATCAATTGATGTT |
| | | TTTATGAATAAATATAAAACTTCAAGCAGAAATAGAGCACTCTCTAATCTAAAAAAA |
| | | GATATATTAAAAGAAGTAATTCTTATTAAAAATTCCAATACAAGCCCTGTAGAAAAA |
| | | AATTTACATTTTGTATGGATAGGTGGAGAAGTCAGTGATATTGCTCTTGAATACATA |
| | | AAACAATGGGCTGATATTAATGCAGAATATAATATTAAACTGTGGTATGATAGTGAA |
| | | GCATTCTTAGTAAATACACTAAAAAAGGCTATAGTTGAATCTTCTACCACTGAAGCA |
| | | TTACAGCTACTAGAGGAAGAGATTCAAAATGCTCAATTTGATAATATGAAATTTTAG |
| | | AAAAAAAGGATGGAATTTATATATGATAGACAAAAAAGGTTTATAAATTATTATAAA |
| | | TCTCAAATCAATAAACCTACAGTACCTACAATAGATGATATTATAAAGTCTCATCTA |
| | | GTATCTGAATATAATAGAGATGAAACTGTATTAGAATCATATAGAACAAATTCTTTG |
| | | AGAAAAATAAATAGTAATCATGGGATAGATATCAGGGCTAATAGTTTGTTTACAGAA |
| | | GAAGAGTTATTAAATATTTATAGTCAGGAGTTGTTAAATCGTGGAAATTTAGCTGCA |
| | | GCATCTGACATAGTAAGATTATTAGCCCTAAAAAATTTTGGCGGAGTATATTTAGAT |
| | | GTTGATATGCTTCCAGGTATTCACTCTGATTTATTTAAAACAATATCTAGACCTAGC |
| | | TCTATTGGACTAGACCGTTGGGAAATGATAAAATTAGAGGGTATTATGAAGTATAAA |
| | | AAATATATAAATAATTATACATCAGAAAACTTTGATAAACTTGATCAACAATTAAAA |
| | | GATAATTTTAAACTCATTATAGAAAGTAAAAGTGAAAAATCTGAGATATTTTCTAAA |
| | | TTAGAAAATTTAAATGTATCTGATCTTGAAATTAAAATAGCTTTCGCTTTAGGCAGT |
| | | GTTATAAATCAAGCCTTGATATCAAAACAAGGTTCATATCTTACTAACCTAGTAATA |
| | | GAACAAGTAAAAAATAGATATCAATTTTTAAACCAACACCTTAACCCAGCCATAGAG |
| | | TCTGATAATAACTTCACAGATACTACTAAAATTTTTCATGATTCATTATTTAATTCA |
| | | GCTACCGCAGAAAACTCTATGTTTTTAACAAAAATAGCACCATACTTACAAGTAGGT |
| | | TTTATGCCAGAAGCTCGCTCCACAATAAGTTTAAGTGGTCCAGGAGCTTATGCGTCA |
| | | GCTTACTATGATTTCATAAATTTACAAGAAAATACTATAGAAAAAACTTTAAAAGCA |
| | | TCAGATTTAATAGAATTTAAATTCCCAGAAAATAATCTATCTCAATTGACAGAACAA |
| | | GAAATAAATAGTCTATGGAGCTTTGATCAAGCAAGTGCAAAATATCAATTTGAGAAA |
| | | TATGTAAGAGATTATACTGGTGGATCTCTTTCTGAAGACAATGGGGTAGACTTTAAT |
| | | AAAAATACTGCCCTCGACAAAAACTATTTATTAAATAATAAAATTCCATCAAACAAT |
| | | GTAGAAGAAGCTGGAAGTAAAAATTATGTTCATTATATCATACAGTTACAAGGAGAT |
| | | GATATAAGTTATGAAGCAACATGCAATTTATTTTCTAAAAATCCTAAAAATAGTATT |
| | | ATTATACAACGAAATATGAATGAAAGTGCAAAAAGCTACTTTTTAAGTGATGATGGA |
| | | GAATCTATTTTAGAATTAAATAAATATAGGATACCTGAAAGATTAAAAAATAAGGAA |
| | | AAAGTAAAAGTAACCTTTATTGGACATGGTAAAGATGAATTCAACACAAGCGAATTT |
| | | GCTAGATTAAGTGTAGATTCACTTTCCAATGAGATAAGTTCATTTTTAGATACCATA |
| | | AAATTAGATATATCACCTAAAAATGTAGAAGTAAACTTACTTGGATGTAATATGTTT |
| | | AGTTATGATTTTAATGTTGAAGAAACTTATCCTGGGAAGTTGCTATTAAGTATTATG |
| | | GACAAAATTACTTCCACTTTACCTGATGTAAATAAAAATTCTATTACTATAGGAGCA |
| | | AATCAATATGAAGTAAGAATTAATAGTGAGGGAAGAAAAGAACTTCTGGCTCACTCA |
| | | GGTAAATGGATAAATAAAGAAGAAGCTATTATGAGCGATTTATCTAGTAAAGAATAC |
| | | ATTTTTTTTGATTCTATAGATAATAAGCTAAAAGCAAAGTCCAAGAATATTCCAGGA |
| | | TTAGCATCAATATGAGAAGATATAAAAACATTATTACTTGATGCAAGTGTTAGTCCT |
| | | GATACAAAATTTATTTTAAATAATCTTAAGCTTAATATTGAATCTTCTATTGGTGAT |
| | | TACATTTATTATGAAAAATTAGAGCCTGTTAAAAATATAATTCACAATTCTATAGAT |
| | | GATTTAATAGATGAGTTCAATCTACTTGAAAATGTATCTGATGAATTATATGAATTA |
| | | AAAAAATTAAATAATCTAGATGAGAAGTATTTAATATCTTTTGAAGATATCTCAAAA |
| | | AATAATTCAACTTACTCTGTAAGATTTATTAACAAAAGTAATGGTGAGTCAGTTTAT |
| | | GTAGAAACAGAAAAAGAAATTTTTTGAAAATATAGCGAACATATTACAAAAGAAATA |
| | | AGTACTATAAAGAATAGTATAATTACAGATGTTAATGGTAATTTATTGGATAATATA |
| | | CAGTTAGATCATACTTCTCAAGTTAATACATTAAACGCAGCATTCTTTATTCAATCA |
| | | TTAATAGATTATAGTAGCAATAAAGATGTACTGAATGATTTAAGTACCTCAGTTAAG |
| | | GTTCAACTTTATGCTCAACTATTTAGTACAGGTTTAAATACTATATATGACTCTATC |
| | | CAATTAGTAAATTTAATATCAAATGCAGTAAATGATACTATAAATGTACTACCTACA |
| | | ATAACAGAGGGGATACCTATTGTATCTACTATATTAGACGGAATAAACTTAGGTGCA |
| | | GCAATTAAGGAATTACTAGACGAACATGACCCATTACTAAAAAAAGAATTAGAAGCT |
| | | AAGGTGGGTGTTTTAGCAATAAATATGTCATTATCTATAGCTGCAACTGTAGCTTCA |
| | | ATTGTTGGAATAGGTGCTGAAGTTACTATTTTCTTATTACCTATAGCTGGTATATCT |
| | | GCAGGAATACCTTCATTAGTTAATAATGAATTAATATTGCATGATAAGGCAACTTCA |
| | | GTGGTAAACTATTTTAATCATTTGTCTGAATCTAAAAAATATGGCCCTCTTAAAACA |
| | | GAAGATGATAAAATTTTAGTTCCTATTGATGATTTAGTAATATCAGAAATAGATTTT |
| | | AATAATAATTCGATAAAACTAGGAACATGTAATATATTAGCAATGGAGGGGGGATCA |
| | | GGACACACAGTGACTGGTAATATAGATCACTTTTTCTCATCTCCATCTATAAGTTCT |
| | | CATATTCCTTCATTATCAATTTATTCTGCAATAGGTATAGAAACAGAAAATCTAGAT |
| | | TTTTCAAAAAAAATAATGATGTTACCTAATGCTCCTTCAAGAGTGTTTTGGTGGGAA |
| | | ACTGGAGCAGTTCCAGGTTTAAGATCATTGGAAAATGACGGAACTAGATTACTTGAT |
| | | TCAATAAGAGATTTATACCCAGGTAAATTTTACTGGAGATTCTATGCTTTTTTCGAT |
| | | TATGCAATAACTACATTAAAACCAGTTTATGAAGACACTAATATTAAAATTAAACTA |
| | | GATAAAGATACTAGAAACTTCATAATGCCAACTATAACTACTAACGAAATTAGAAAC |
| | | AAATTATCTATTCATTTGATGGAGCAGGAGGAACTTACTCTTTATTATTATCTTCAT |
| | | ATCCAATATCAACGAATATAAATTTATCTAAAGATGATTTATGGATATTTAATATTG |
| | | ATAATGAAGTAAGAGAAATATCTATAGAAAATGGTACTATTAAAAAAGGAAAGTTAA |
| | | TAAAAGATGTTTTAAGTAAAATTGATATAAATAAAAATAAACTTATTATAGGCAATC |
| | | AAACAATAGATTTTTCAGGCGATATAGATAATAAAGATAGATATATATTCTTGACTT |
| | | GTGAGTTAGATGATAAAATTAGTTTAATAATAGAAATAAATCTTGTTGCAAAATCTT |
| | | ATAGTTTGTTATTGTCTGGGGATAAAAATTATTTGATATCCAATTTATCTAATACTA |
| | | TTGAGAAAATCAATACTTTAGGCCTAGATAGTAAAAATATAGCGTACAATTACACTG |
| | | ATGAATCTAATAATAAATATTTTGGAGCTATATCTAAAACAAGTCAAAAAAGCATAA |
| | | TACATTATAAAAAAGACAGTAAAAATATATTAGAATTTTATAATGACAGTACATTAG |
| | | AATTTAACAGTAAAGATTTTATTGCTGAAGATATAAATGTATTTATGAAAGATGATA |
| | | TTAATACTATAACAGGAAAATACTATGTTGATAATAATACTGATAAAAGTATAGATT |
| | | TCTCTATTTCTTTAGTTAGTAAAAATCAAGTAAAAGTAAATGGATTATATTTAAATG |
| | | AATCCGTATACTCATCTTACCTTGATTTTGTGAAAAATTCAGATGGACACCATAATA |
| | | CTTCTAATTTTATGAATTTATTTTTGGACAATATAAGTTTCTGGAAATTGTTTGGGT |
| | | TTGAAAATATAAATTTTGTAATCGATAAATACTTTACCCTTGTTGGTAAAACTAATC |
| | | TTGGATATGTAGAATTTATTTGTGACAATAATAAAAATATAGATATATATTTTGGTG |
| | | AATGGAAAACATCGTCATCTAAAAGCACTATATTTAGCGGAAATGGTAGAAATGTTG |
| | | TAGTAGAGCCTATATATAATCCTGATACGGGTGAAGATATATCTACTTCACTAGATT |
| | | TTTCCTATGAACCTCTCTATGGAATAGATAGATATATAAATAAAGTATTGATAGCAC |
| | | CTGATTTATATACAAGTTTAATAAATATTAATACCAATTATTATTCAAATGAGTACT |
| | | ACCCTGAGATTATAGTTCTTAACCCAAATACATTCCACAAAAAAGTAAATATAAATT |
| | | TAGATAGTTCTTCTTTTGAGTATAAATGGTCTACAGAAGGAAGTGACTTTATTTTAG |
| | | TTAGATACTTAGAAGAAAGTAATAAAAAAATATTACAAAAAATAAGAATCAAAGGTA |
| | | TCTTATCTAATACTCAATCATTTAATAAAATGAGTATAGATTTTAAAGATATTAAAA |
| | | AACTATCATTAGGATATATAATGAGTAATTTTAAATCATTTAATTCTGAAAATGAAT |
| | | TAGATAGAGATCATTTAGGATTTAAAATAATAGATAATAAAACTTATTACTATGATG |
| | | AAGATAGTAAATTAGTTAAAGGATTAATCAATATAAATAATTCATTATTCTATTTTG |
| | | ATCCTATAGAATTTAACTTAGTAACTGGATGGCAAACTATCAATGGTAAAAAATATT |
| | | ATTTTGATATAAATACTGGAGCAGCTTTAACTAGTTATAAAATTATTAATGGTAAAC |
| | | ACTTTTATTTTAATAATGATGGTGTGATGCAGTTGGGAGTATTTAAAGGACCTGATG |
| | | GATTTGAATATTTTGCACCTGCCAATACTCAAAATAATAACATAGAAGGTCAGGCTA |
| | | TAGTTTATCAAAGTAAATTCTTAACTTTGAATGGCAAAAAATATTATTTTGATAATG |
| | | ACTCAAAAGCAGTCACTGGATGGAGAATTATTAACAATGAGAAATATTACTTTAATC |
| | | CTAATAATGCTATTGCTGCAGTCGGATTGCAAGTAATTGACAATAATAAGTATTATT |
| | | TCAATCCTGACACTGCTATCATCTCAAAAGGTTGGCAGACTGTTAATGGTAGTAGAT |
| | | ACTACTTTGATACTGATACCGCTATTGCCTTTAATGGTTATAAAACTATTGATGGTA |
| | | AACACTTTTATTTTGATAGTGATTGTGTAGTGAAAATAGGTGTGTTTAGTACCTCTA |
| | | ATGGATTTGAATATTTTGCACCTGCTAATACTTATAATAATAACATAGAAGGTCAGG |
| | | CTATAGTTTATCAAAGTAAATTCTTAACTTTGAATGGTAAAAAATATTACTTTGATA |
| | | ATAACTCAAAAGCAGTTACCGGATGGCAAACTATTGATAGTAAAAAATATTACTTTA |
| | | ATACTAACACTGCTGAAGCAGCTACTGGATGGCAAACTATTGATGGTAAAAAATATT |
| | | ACTTTAATACTAACACTGCTGAAGCAGCTACTGGATGGCAAACTATTGATGGTAAAA |
| | | AATATTACTTTAATACTAACACTGCTATAGCTTCAACTGGTTATACAATTATTAATG |
| | | GTAAACATTTTTATTTTAATACTGATGGTATTATGCAGATAGGAGTGTTTAAAGGAC |
| | | CTAATGGATTTGAATATTTTGCACCTGCTAATACGGATGCTAACAACATAGAAGGTC |
| | | AAGCTATACTTTACCAAAATGAATTCTTAACTTTGAATAGTAAAAAATATTACTTTG |
| | | GTAGTGACTCAAAAGCAGTTACTGGATGGAGAATTATTAACAATAAGAAATATTACT |
| | | TTAATCCTAATAATGCTATTGCTGCAATTCATCTATGCACTATAAATAATGACAAGT |
| | | ATTACTTTAGTTATGATGGAATTCTTCAAAATGGATATATTACTATTGAAAGAAATA |
| | | ATTTCTATTTTGATGCTAATAATGAATCTAAAATGGTAACAGGAGTATTTAAAGGAC |
| | | CTAATGGATTTGAGTATTTTGCACCTGCTAATACTCACAATAATAACATAGAAGGTC |
| | | AGGCTATAGTTTACCAGAACAAATTCTTAACTTTGAATGGCAAAAAATATTATTTTG |
| | | ATAATGACTCAAAAGCAGTTACTGGATGGCAAACCATTGATGGTAAAAAATATTACT |
| | | TTAATCTTAACACTGCTGAAGCAGCTACTGGATGGCAAACTATTGATGGTAAAAAAT |
| | | ATTACTTTAATCTTAACACTGCTGAAGCAGCTACTGGATGGCAAACTATTGATGGTA |
| | | AAAAATATTACTTTAATACTAACACTTTCATAGCCTCAACTGGTTATACAAGTATTA |
| | | ATGGTAAACATTTTTATTTTAATACTGATGGTATTATGCAGATAGGAGTGTTTAAAG |
| | | GACCTAATGGATTTGAATACTTTGCACCTGCTAATACTCATAATAATAACATAGAAG |
| | | GTCAAGCTATACTTTACCAAAATAAATTCTTAACTTTGAATGGTAAAAAATATTACT |
| | | TTGGTAGTGACTCAAAAGCAGTTACCGGATTGCGAACTATTGATGGTAAAAAATATT |
| | | ACTTTAATACTAACACTGCTGTTGCAGTTACTGGATGGCAAACTATTAATGGTAAAA |
| | | AATACTACTTTAATACTAACACTTCTATAGCTTCAACTGGTTATACAATTATTAGTG |
| | | GTAAACATTTTTATTTTAATACTGATGGTATTATGCAGATAGGAGTGTTTAAAGGAC |
| | | CTGATGGATTTGAATACTTTGCACCTGCTAATACAGATGCTAACAATATAGAAGGTC |
| | | AAGCTATACGTTATCAAAATAGATTCCTATATTTACATGACAATATATATTATTTTG |
| | | GTAATAATTCAAAAGCGGCTACTGGTTGGGTAACTATTGATGGTAATAGATATTACT |
| | | TCGAGCCTAATACAGCTATGGGTGCGAATGGTTATAAAACTATTGATAATAAAAACT |
| | | TTTACTTTAGAAATGGTTTACCTCAGATAGGAGTGTTTAAAGGGTCTAATGGATTTG |
| | | AATACTTTGCACCTGCTAATACGGATGCTAACAATATAGAAGGTCAAGCTATACGTT |
| | | ATCAAAATAGATTCCTACATTTACTTGGAAAAATATATTACTTTGGTAATAATTCAA |
| | | AAGCAGTTACTGGATGGCAAACTATTAATGGTAAAGTATATTACTTTATGCCTGATA |
| | | CTGCTATGGCTGCAGCTGGTGGACTTTTCGAGATTGATGGTGTTATATATTTCTTTG |
| | | GTGTTGATGGAGTAAAAGCCCCTGGGATATATGGCTAA |
| 8 | tcdB from GenBank | ATGAGTTTAGTTAATAGAAAACAGTTAGAAAAAATGGCAAATGTAAGATTTCGTACT |
| | KC292190 | CAAGAAGATGAATATGTTGCAATATTGGATGCTTTAGAAGAATATCATAATATGTCA |
| | | GAGAATACTGTAGTCGAAAAATATTTAAAATTAAAAGATATAAATAGTTTAACAGAT |
| | | ATTTATATAGATACATATAAAAAATCTGGTAGAAATAAAGCCTTAAAAAAATTTAAG |
| | | GAATATCTAGTTACAGAAGTATTAGAGCTAAAGAATAATAATTTAACTCCAGTTGAG |
| | | AAAAATTTACATTTGTTTGGATTGGAGGTCAAATAAATGACACTGCTATTAATTATA |
| | | TAAATCAATGGAAAGATGTAAATAGTGATTATAATGTTAATGTTTTTTATGATAGTA |
| | | ATGCATTTTTGATAAACACATTGAAAAAAACTGTAGTAGAATCAGCAATAAATGATA |
| | | CACTTGAATCATTTAGAGAAAACTTAAATGACCCTAGATTTGACTATAATAAATTCT |
| | | TCAGAAAACGTATGGAAATAATTTATGATAAACAGAAAAATTTCATAAACTACTATA |
| | | AAGCTCAAAGAGAAGAAAATCCTGAACTTATAATTGATGATATTGTAAAGACATATC |
| | | TTTCAAATGAGTATTCAAAGGAGATAGATGAACTTAATACCTATATTGAAGAATCCT |
| | | TAAATAAAATTACACAGAATAGTGGAAATGATGTTAGAAACTTTGAAGAATTTAAAA |
| | | ATGGAGAGTCATTCAACTTATATGAACAAGAGTTGGTAGAAAGGTGGAATTTAGCTG |
| | | CTGCTTCTGACATATTAAGAATATCTGCATTAAAAGAAATTGGTGGTATGTATTTAG |
| | | ATGTTGATATGTTACCAGGAATACAACCAGACTTATTTGAGTCTATAGAGAAACCTA |
| | | GTTCAGTAACAGTGGATTTTTGGGAAATGACAAAGTTAGAAGCTATAATGAAATACA |
| | | AAGAATATATACCAGAATATACCTCAGAACATTTTGACATGTTAGACGAAGAAGTTC |
| | | AAAGTAGTTTGAATCTGTTCTAGCTTCTAAGTCAGATAAATCAGAAATATTCTCATC |
| | | ACTTGGTGATATGGAGGCATCACCACTAGAAGTTAAAATTGCATTTAATAGTAAGGG |
| | | TATTATAAATCAAGGGCTAATTTCTGTGAAAGACTCATATTGTAGCAATTTAATAGT |
| | | AAAACAAATCGAGAATAGATATAAAATATTGAATAATAGTTTAAATCCAGCTATTAG |
| | | CGAGGATAATGATTTTAATACTACAACGAATACCTTTATTGATAGTATAATGGCTGA |
| | | AGCTAATGCAGATAATGGTAGATTTATGATGGAACTAGGAAAGTATTTAAGAGTTGG |
| | | TTTCTTCCCAGATGTTAAAACTACTATTAACTTAAGTGGCCCTGAAGCATATGCGGC |
| | | AGCTTATCAAGATTTATTAATGTTTAAAGAAGGCAGTATGAATATCCATTTGATAGA |
| | | AGCTGATTTAAGAAACTTTGAAATCTCTAAAACTAATATTTCTCAATCAACTGAACA |
| | | AGAAATGGCTAGCTTATGGTCATTTGACGATGCAAGAGCTAAAGGTCAATTTGAAGA |
| | | ATATAAAAGGAATTATTTTGAAGGTTCTCTTGGTGAAGATGATAATCTTGATTTTTC |
| | | TCAAAATATAGTAGTTGACAAGGAGTATCTTTTAGAAAAAATATCTTCATTAGCAAG |
| | | AAGTTCAGAGAGAGGATATATACACTATATTGTTCAGTTACAAGGAGATAAAATTAG |
| | | TTATGAAGCAGCATGTAACTTATTTGCAAAGACTCCTTATGATAGTGTACTGTTTCA |
| | | GAAAAATATAGAAGATTCAGAAATTGCATATTATTATAATCCTGGAGATGGTGAAAT |
| | | ACAAGAAATAGACAAGTATAAAATTCCAAGTATAATTTCTGATAGACCTAAGATTAA |
| | | ATTAACATTTATTGGTCATGGTAAAGATGAATTTAATACTGATATATTTGCAGGTTT |
| | | TGATGTAGATTCATTATCCACAGAAATAGAAGCAGCAATAGATTTAGCTAAAGAGGA |
| | | TATTTCTCCTAAGTCAATAGAAATAAATTTATTAGGATGTAATATGTTTAGCTACTC |
| | | TATCAACGTAGAGGAGACTTATCCTGGAAAATTATTACTTAAAGTTAAAGATAAAAT |
| | | ATCAGAATTAATGCCATCTATAAGTCAAGACTCTATTATAGTAAGTGCAAATCAATA |
| | | TGAAGTTAGAATAAATAGTGAAGGAAGAAGAGAATTATTGGATCATTCTGGTGAATG |
| | | GATAAATAAAGAAGAAAGTATTATAAAGGATATTTCATCAAAAGAATATATATCATT |
| | | TAATCCTAAAGAAAATAAAATTACAGTAAAATCTAAAAATTTACCTGAGCTATCTAC |
| | | ATTATTACAAGAAATTAGAAATAATTCTAATTCAAGTGATATTGAACTAGAAGAAAA |
| | | AGTAATGTTAACAGAATGTGAGATAAATGTTATTTCAAATATAGATACGCAAATTGT |
| | | TGAGGAAAGGATTGAAGAAGCTAAGAATTTAACTTCTGACTCTATTAATTATATAAA |
| | | AGATGAATTTAAACTAATAGAATCTATTTCTGATGCACTATGTGACTTAAAACAACA |
| | | GAATGAATTAGAAGATTCTCATTTTATATCTTTTGAGGACATATCAGAGACTGATGA |
| | | GGGATTTAGTATAAGATTTATTAATAAAGAAACTGGAGAATCTATATTTGTAGAAAC |
| | | TGAAAAAACAATATTCTCTGAATATGCTAATCATATAACTGAAGAGATTTCTAAGAT |
| | | AAAAGGTACTATATTTGATACTGTAAATGGTAAGTTAGTAAAAAAAGTAAATTTAGA |
| | | TACTACACACGAAGTAAATACTTTAAATGCTGCATTTTTTATACAATCATTAATAGA |
| | | ATATAATAGTTCTAAAGAATCTCTTAGTAATTTAAGTGTAGCAATGAAAGTCCAAGT |
| | | TTACGCTCAATTATTTAGTACTGGTTTAAATACTATTACAGATGCAGCCAAAGTTGT |
| | | TGAATTAGTATCAACTGCATTAGATGAAACTATAGACTTACTTCCTACATTATCTGA |
| | | AGGATTACCTATAATTGCAACTATTATAGATGGTGTAAGTTTAGGTGCAGCAATCAA |
| | | AGAGCTAAGTGAAACGAGTGACCCATTATTAAGACAAGAAATAGAAGGTAAGATAGG |
| | | TATAATGGCAGTAAATTTAACAACAGCTACAACTGCAATGATTACTTCATCTTTGGG |
| | | GATAGCTAGTGGATTTAGTATACTTTTAGTTCCTTTAGCAGGAATTTCAGCAGGTAT |
| | | ACCAAGCTTAGTAAACAATGAACTTGTACTTCGAGATAAGGCAACAAAGGTTGTAGA |
| | | TTATTTTAAACATGTTTCATTAGTTGAAACTGAAGGAGTATTTACTTTATTAGATGA |
| | | TAAAATAATGATGCCACAAGATGATTTAGTGATATCAGAAATAGATTTTAATAATAA |
| | | TTCAATAGTTTTAGGTAAATGTGAAATCTGGAGAATGGAAGGTGGTTCAGGTCATAC |
| | | TGTAACTGATGATATAGATCACTTCTTTTCAGCACCATCAATAACATATAGAGAGCC |
| | | ACACTTATCTATATATGACGTATTGGAAGTACAAAAAGAAGAACTTGATTTGTCAAA |
| | | AGATTTAATGGTATTACCTAATGCTCCAAATAGAGTATTTGCTTGGGAAACAGGATG |
| | | GACACCAGGTTTAAGAAGCTTAGAAAATGATGGCACAAAACTGTTAGACCGTATAAG |
| | | AGATAACTATGAAGGTGAGTTTTATTGGAGATATTTTGCTTTTATAGCTGATGCTTT |
| | | AATAACAACATTAAAACCAAGATATGAAGATACTAATATAAGAATAAATTTAGATAG |
| | | TAATACTAGAAGTTTTATAGTTCCAATAATAACTACAGAATATATAAGAGAAAAATT |
| | | ATCATATTCTTTCTATGGTTCAGGAGGAACTTATGCATTGTCTCTTTCTCAATATAA |
| | | TATGGGTATAAATATAGAATTAAGTGAAAGTGATGTTTGGATTATAGATGTTGATAA |
| | | TGTTGTGAGAGATGTAACTATAGAATCTGATAAAATTAAAAAAGGTGATTTAATAGA |
| | | AGGTATTTTATCTACACTAAGTATTGAAGAGAATAAAATTATCTTAAATAGCCATGA |
| | | GATTAATTTTCTGGTGAGGTAAATGGAAGTAATGGATTTGTTTCTTTAACATTTTCA |
| | | ATTTTAGAAGGAATAAATGCAATTATAGAAGTTGATTTATTATCTAAATCATATAAA |
| | | TTACTTATTTCTGGCGAATTAAAAATATTGATGTTAAATTCAAATCATATTCAACAG |
| | | AAAATAGATTATATAGGATTCAATAGCGAATTACAGAAAAATATACCATATAGCTTT |
| | | GTAGATAGTGAAGGAAAAGAGAATGGTTTTATTAATGGTTCAACAAAAGAAGGTTTA |
| | | TTTGTATCTGAATTACCTGATGTAGTTCTTATAAGTAAGGTTTATATGGATGATAGT |
| | | AAGCCTTCATTTGGATATTATAGTAATAATTTGAAAGATGTCAAAGTTATAACTAAA |
| | | GATAATGTTAATATATTAACAGGTTATTATCTTAAGGATGATATAAAAATCTCTCTT |
| | | TCTTTGACTCTACAAGATGAAAAAACTATAAAGTTAAATAGTGTGCATTTAGATGAA |
| | | AGTGGAGTAGCTGAGATTTTGAAGTTCATGAATAGAAAAGGTAATACAAATACTTCA |
| | | GATTCTTTAATGAGCTTTTTAGAAAGTATGAATATAAAAAGTATTTTCGTTAATTTC |
| | | TTACAATCTAATATTAAGTTTATATTAGATGCTAATTTTATAATAAGTGGTACTACT |
| | | TCTATTGGCCAATTTGAGTTTATTTGTGATGAAAATGATAATATACAACCATATTTC |
| | | ATTAAGTTTAATACACTAGAAACTAATTATACTTTATATGTAGGAAATAGACAAAAT |
| | | ATGATAGTGGAACCAAATTATGATTTAGATGATTCTGGAGATATATCTTCAACTGTT |
| | | ATCAATTTCTCTCAAAAGTATCTTTATGGAATAGACAGTTGTGTTAATAAAGTTGTA |
| | | ATTTCACCAAATATTTATACAGATGAAATAAATATAACGCCTGTATATGAAACAAAT |
| | | AATACTTATCCAGAAGTTATTGTATTAGATGCAAATTATATAAATGAAAAAATAAAT |
| | | GTTAATATCAATGATCTATCTATACGATATGTATGGAGTAATGATGGTAATGATTTT |
| | | ATTCTTATGTCAACTAGTGAAGAAAATAAGGTGTCACAAGTTAAAATAAGATTCGTT |
| | | AATGTTTTTAAAGATAAGACTTTGGCAAATAAGCTATCTTTAACTTTAGTGATAAAC |
| | | AAGATGTACCTGTAAGTGAAATAATCTTATCATTTACACCTTCATATTATGAGGATG |
| | | GATTGATTGGCTATGATTTGGGTCTAGTTTCTTTATATAATGAGAAATTTTATATTA |
| | | ATAACTTTGGAATGATGGTATCTGGATTAATATATATTAATGATTCATTATATTATT |
| | | TTAAACCACCAGTAAATAATTTGATAACTGGATTTGTGACTGTAGGCGATGATAAAT |
| | | ACTACTTTAATCCAATTAATGGTGGAGCTGCTTCAATTGGAGAGACAATAATTGATG |
| | | ACAAAAATTATTATTTCAACCAAAGTGGAGTGTTACAAACAGGTGTATTTAGTACAG |
| | | AAGATGGATTTAAATATTTTGCCCCAGCTAATACACTTGATGAAAACCTAGAAGGAG |
| | | AAGCAATTGATTTTACTGGAAAATTAATTATTGACGAAAATATTTATTATTTTGATG |
| | | ATAATTATAGAGGAGCTGTAGAATGGAAAGAATTAGATGGTGAAATGCACTATTTTA |
| | | GCCCAGAAACAGGTAAAGCTTTTAAAGGTCTAAATCAAATAGGTGATGATAAATACT |
| | | ATTTCAATTCTGATGGAGTTATGCAAAAAGGATTTGTTAGTATAAATGATAATAAAC |
| | | ACTATTTTGATGATTCTGGTGTTATGAAAGTAGGTTACACTGAAATAGATGGCAAGC |
| | | ATTTCTACTTTGCTGAAAACGGAGAAATGCAAATAGGAGTATTTAATACAGAAGATG |
| | | GATTTAAATATTTTGCTCATCATAATGAAGATTTAGGAAATGAAGAAGGTGAAGAAA |
| | | TCTCATATTCTGGTATATTAAATTTCAATAATAAAATTTACTATTTTGATGATTCAT |
| | | TTACAGCTGTAGTTGGATGGAAAGATTTAGAGGATGGTTCAAAGTATTATTTTGATG |
| | | AAGATACAGCAGAAGCATATATAGGTTTGTCATTAATAAATGATGGTCAATATTATT |
| | | TTAATGATGATGGAATTATGCAAGTTGGATTTGTCACTATAAATGATAAAGTCTTCT |
| | | ACTTTTCTGACTCTGGAATTATAGAATCTGGAGTACAAAACATAGATGACAATTATT |
| | | TCTATATAGATGATAATGGTATAGTTCAAATTGGTGTATTTGATACTTCAGATGGAT |
| | | ATAAATATTTTGCACCTGCTAATACTGTAAATGATAATATTTACGGACAAGCAGTTG |
| | | AATATAGTGGTTTAGTTAGAGTTGGGGAAGATGTATATTATTTTGGAGAAACATATA |
| | | CAATTGAGACTGGATGGATATATGATATGGAAAATGAAAGTGATAAATATTATTTCA |
| | | ATCCAGAAACTAAAAAAGCATGCAAAGGTATTAATTTAATTGATGATATAAAATATT |
| | | ATTTTGATGAGAAGGGCATAATGAGAACGGGTCTTATATCATTTGAAAATAATAATT |
| | | ATTACTTTAATGAGAATGGTGAAATGCAATTTGGTTATATAAATATAGAAGATAAGA |
| | | TGTTCTATTTTGGTGAAGATGGTGTCATGCAGATTGGAGTATTTAATACACCAGATG |
| | | GATTTAAATACTTTGCACATCAAAATACTTTGGATGAGAATTTTGAGGGAGAATCAA |
| | | TAAACTATACTGGTTGGTTAGATTTAGATGAAAAGAGATATTATTTTACAGATGAAT |
| | | ATATTGCAGCAACTGGTTCAGTTATTATTGATGGTGAGGAGTATTATTTTGATCCTG |
| | | ATACAGCTCAATTAGTGATTAGTGAATAG |
| 9 | cdtA from GenBank | ATGAAAAAATTTAGAAAACATAAAAGTATTAGTAATTGTATATCTATATTGTTGATA |
| | HQ639679 | TTATATCTAACTTTAGGTAGTTTGTTACCTAATAACATTTATGCACAAGACTTACAA |
| | | AGCTATAGTGAAAAAGTTTGCAATACTACTTACAAGGCTCCTATAGAAAGACCAGAA |
| | | GATTTTCTTAAAGATAAAGAAAGGGCTAAAGAATGGGAAAGAAAAGAAGCAGAAAGA |
| | | ATAGAGCAAAAACTTGAAAGATCTGAAAAAGAAGCATTAGAATCATATAAAAAAGAT |
| | | TCTGTAGAAATAAATAAATATTCTCAGACAAGAAATTATTTTTATGATTATCAAATA |
| | | GAAGCAAATTCTCGAGAAAAAGAATATAGAGAACTTCGAAATGCTATATCAAAAAAT |
| | | AAAATAGATAAACCTATGTATGTCTATTATTTTGAATCTCCAGAAAAATTTGCATTT |
| | | AATAAAGTAATAAGAACAGAAAATCAAAACGAAATTTCATTAGAAAAATTTAATGAG |
| | | TTTAAAGAAACTATACAAAACAAATTATTTAAGCAAGATGGATTTAAAGAAATTTCT |
| | | TTATATGAACCTGGAAAAGGTGATGAAGAACCTACACCATTACTTATGCACTTAAAA |
| | | TTACCTAGAAATACTGGTATGTTACCATATACAAATACTAACAATGTAAGTACATTA |
| | | ATAGAGCAAGGATATAGTATAAAAATAGATAAAATTGTTCGTATAGTTATAGATGGG |
| | | AAACATTATATTAAAGCAGAAGCATCTGTTGTAAGTAGTCTTGATTTTAAGGATGAT |
| | | GTAAGTAAGGGGGACTCTTGGGGTAAAGCAAATTATAATGATTGGAGTAATAAATTA |
| | | ACACCTAATGAACTTGCTGATGTAAATGATTATATGCGTGGAGGATATACTGCAATT |
| | | AATAATTATTTAATATCAAATGGTCCAGTAAATAACCCTAACCCAGAATTAGATTCT |
| | | AAAATCACAAACATTGAAAATGCATTAAAACGTGAACCTATTCCAACTAATTTAACT |
| | | GTATATAGAAGATCTGGTCCTCAAGAATTTGGTTTAACCCTTACTTCCCCTGAATAT |
| | | GATTTTAACAAACCAGAAAATATAGATGCTTTTAAATCAAAATGGGAAGGACAAACA |
| | | CTGTCTTATCCAAACTTTATTAGTACTAGTATTGGTAGTGTGAATATGAGTGCATTT |
| | | GCTAAAAGAAAAATAGTACTACGTATAACTATACCTAAAGGTTCTCCTGGAGCCTAT |
| | | CTATCAGCTATTCCAGGTTATGCAGGTGAATATGAAGTACTTTTAAATCATGGAAGC |
| | | AAATTTAAAATCAGTAAAATTGATTCTTACAAAGATGGCGCTATAACAAAATTAATT |
| | | GTTGATGCAACATTGATACCTTAA |

FRET cassettes

| | | |
|---|---|---|
| 10 | Exemplary cdtB | Red-TCT-Ecl-AGCCGGTTTTCCGGCTGAGACGTCCGTGGCCT-Hexanediol |
| | FRET cassette | |
| 11 | Exemplary tcdC | HEX-TCT-BBQdT-AGCCGGTTTTCCGGCTGAGCCTCGGCGCG-Hexanediol |
| | FRET cassette | |
| 12 | Exemplary | FAM-TCT-BBQdT-AGCCGGTTTTCCGGCTGAGACTCCGCGTCCGT-Hexanediol |
| | tcdB/tcdA | |
| | FRET cassette | |

Binary Toxin oligomers

| | | |
|---|---|---|
| 13 | cdtA Forward Primer | GAGCAAGGATATAGTAAAAATAGATAAAATTGTTCGTATAGTTATAGATGG |
| 14 | cdtA Invader | TATATTAAAGCAGAAGCATCTGTTGTAAGTAGTCTTGt |
| 15 | cdtA Invader | TATATTAAAGCAGAAGCATCTGTTGTAAGTAGTCTTGc |
| 16 | THS for cdtA probe | ATTTTAAGGATGATGTAAGTAAGGGAG |
| 17 | THS for cdtA probe | CGATTTTAAGGATGATGTAAGTAAGGG |
| 18 | cdtA Primary Probe | CGCGAGGCCGATTTTAAGGATGATGTAAGTAAGGGAG-hexanediol |
| 19 | cdtA Primary Probe | AGGCCACGGACGATTTTAAGGATGATGTAAGTAAGGGAG-hexanediol |
| 20 | cdtA Primary Probe | AGGCCACGGACGATTTTAAGGATGATGTAAGTAAGGG-hexanediol |
| 21 | cdtA Reverse Primer | ACTCCAATCACTATAATTTGCTTTACCCCAAG |
| 22 | cdtA Reverse Primer | CGCATATAATCATTTACATCAGCAAGTTCATTAGGT |
| 23 | cdtA Reverse Primer | ATTTGCTTTACCCCAAGAGTCTCCC |
| 24 | cdtA Reverse Primer | GCTTTACCCCAAGAGTCTCCC |
| 25 | cdtA Reverse Primer | ACTCCAATCACTATAATTTGCTTTACCCCAAGAGTC |
| 26 | cdtA Reverse Primer | CAATCACTATAATTTGCTTTACCCCAAGAGTC |
| 27 | cdtA Reverse Primer | AGGTGTTAATTTATTACTCCAATCACTATAATTTGCTTTACC |
| 28 | cdtA Reverse Primer | TGTTAATTTATTACTCCAATCACTATAATTTGCTTTACC |
| 29 | cdtA Reverse Primer | GTTCATTAGGTGTTAATTTATTACTCCAATCACTATAATTTGC |
| 30 | Forward Primer | GTTGATGTCTGATTGGGAAGACGAAGATTTGG |
| 31 | Forward Primer | TTTGACCCAAGGTTGATGTCTGATTGG |
| 32 | Forward Primer | GACCCAAGGTTGATGTCTGATTGG |
| 33 | Forward Primer | CTTTGACCCAAGGTTGATGTCTGATTGG |
| 34 | THS for cdtB probe | CGTTCATATGAATCTGGTATATTATCATTATC |
| 35 | THS for cdtB probe | GTTCATATGAATCTGGTATATTATCATTATCT |
| 36 | THS for cdtB probe | CGTTCATATGAATCTGGTATATTATC |
| 37 | cdtB Primary Probe | aggccacggacgCGTTCATATGAATCTGGTATATTATCATTATC-hexanediol |
| 38 | cdtB Primary Probe | aggccacggacgGTTCATATGAATCTGGTATATTATCATTATCT-hexanediol |
| 39 | cdtB Primary Probe | aggccacggacgCGTTCATATGAATCTGGTATATTATC-hexanediol |
| 40 | cdtB Reverse iPrimer | CACTTAACTGCAATTAAGTCCTTAATAGTATATCCATTTC |
| 41 | cdtB Reverse iPrimer | ACTTAACTGCAATTAAGTCCTTAATAGTATATCCATTTCG |
| 42 | cdtB Invader | ACTTAACTGCAATTAAGTCCTTAATAGTATATCCATTTCt |
| 43 | THS for cdtB probe | GGAAGACGAAGATTTGGATAC |
| 44 | THS for cdtB probe | GATACAGATAATGATAATATACCAGATTCATAT |
| 45 | THS for cdtB probe | GTTCATATGAATCTGGTATATTATCATTATCT |
| 46 | cdtB Primary Probe | CGCGAGGCCGGGAAGACGAAGATTTGGATAC-hexanediol |
| 47 | cdtB Primary Probe | CGCGAGGCCGGATACAGATAATGATAATATACCAGATTCATAT-hexanediol |
| 48 | cdtB Primary Probe | CGCGAGGCCGGTTCATATGAATCTGGTATATTATCATTATCT-hexanediol |
| 49 | cdtB Reverse Primer | CATAATCTGTATATGGATCTCCAGCAGTATTTGACTC |
| 50 | cdtB Reverse Primer | GACTCTAAATAATTTGATACATATTTCTTATAGCCTTGTTCTGC |
| 51 | cdtB Primary Probe | GGAAGACGAAGATTTGGATAC |
| 52 | cdtB Primary Probe | GGAAGACGAAGATTTGGATACA |
| 53 | cdtB Primary Probe | AGGCCACGGACGGGAAGACGAAGATTTGGATAC-hexanediol |
| 54 | cdtB Primary Probe | AGGCCACGGACGGGAAGACGAAGATTTGGATACA-hexanediol |
| 55 | cdtB Froward Primer | GATCCATTTATCCCAAATAAC |
| 56 | cdtB Reverse Primer | CATATTTCTTATAGCCTTGTTCTG |

tcdA oligomers

| | | |
|---|---|---|
| 57 | THS for tcdA Probe | GAATAAACTTAGGTGCAGCAA |
| 58 | tcdA Primary Probe | TCCGCGCGTCCGAATAAACTTAGGTGCAGCAA-hexanediol |
| 59 | tcdA Forward Primer | CTTTATGCTCAACTATTTAGTAC |
| 60 | tcdA Forward Primer | CAGTAAATGATACTATAAATGTACTAC |
| 61 | THS for tcdA Probe | CTAGACGAACATGACCCAT |
| 62 | tcdA Primary Probe | acggacgcggagCTAGACGAACATGACCCAT-hexanediol |
| 63 | tcdA Invader | ATAAACTTAGGTGCAGCAATTAAGGAATTAa |
| 64 | tcdA Forward iPrimer | CAGAGGGGATACCTATTGTATCTACTATATTAGACGG |
| 65 | tcdA Forward iPrimer | GAGGGGATACCTATTGTATCTACTATTAGACGG |
| 66 | THS for tcdA Probe | CTAGACGAACATGACCCAT |
| 67 | THS for tcdA Probe | GAATAAACTTAGGTGCAGCAA |
| 68 | tcdA Primary Probe | cgcgccgaggCTAGACGAACATGACCCAT-hexanediol |
| 69 | tcdA Primary Probe | acggacgcggagGAATAAACTTAGGTGCAGCAA-hexanediol |
| 70 | tcdA Reverse Primer | GTAACTTCAGCACCTATTCCAACAATTGAAGCTAC |
| 71 | tcdA Reverse Primer | AGTAATGGGTCATGTTCGTCTAGTAATTCC |
| 72 | tcdA Reverse Primer | GACATATTTATTGCTAAAACACCCACCTTAGCTTC |
| 73 | tcdA Reverse Primer | GATATACCAGCTATAGGTAATAAG |

tcdC oligomers

| | | |
|---|---|---|
| 74 | tcdC 117 Invader | GGTGAGGATATATTGCCAAAAAACACGCg |
| 75 | tcdC 117 Invader | ACAAAGGGTATTGCTCTACTGGCATTTATTTTc |
| 76 | tcdC 117 Iprimer | ACAAAGGGTATTGCTCTACTGGCATTTATTTTG |
| | (N = A, C, T, or G) | |
| 77 | Not Used | |
| 78 | Not Used | |
| 79 | Not Used | |
| 80 | Not Used | |
| 81 | Not Used | |
| 82 | Not Used | |
| 83 | Not Used | |
| 84 | Not Used | |
| 85 | Not Used | |
| 86 | tcdC 117del Invader | GGTGAGGATATTGCCAAAAAACACACg |
| 87 | tcdC 117del Revese | GGTGAGGATATATTGCCAAAAAACACACCA |
| | iPrimer | |
| 88 | tcdC 117del Revese | GGTGAGGATATATTGCCAAAAAACACACC |
| | iPrimer | |
| 89 | tcdC 117del Revese | GGTGAGGATATATTGCCAAAAAACACGC |
| | iPrimer | |
| 90 | tcdC 117del Revese | GGTGAGGATATATTGCCAAAAAACACAC |
| | iPrimer | |
| 91 | tcdC 117del Revese | GGTGAGGATATATTGCCAAAAAACACG |
| | iPrimer | |
| 92 | tcdC 117del Revese | GGTGAGGATATATTGCCAAAAAACACA |
| | iPrimer | |
| 93 | THS for tcdC | CAAAATAAATGCCAGTAGAGC |
| | 117del Probe | |
| 94 | THS for tcdC | AAAATAAATGCCAGTAGAGCAATAT |
| | 117del Probe | |
| 95 | THS for tcdC | AAAATAAATGCCAGTAGAGCAATATC |
| | 117del Probe | |
| 96 | THS for tcdC | CAAAATAAATGCCAGTAGAG |
| | 117del Probe | |
| 97 | THS for tcdC | CAAAATAAATGCCAGTAGA |
| | 117del Probe | |
| 98 | THS for tcdC | CAAAATAAATGCCAGTAGAGCAA |
| | 117del Probe | |
| 99 | THS for tcdC | CAAAATAAATGCCAGTAGAGCAATAC |
| | 117del Probe | |
| 100 | THS for tcdC | CAAATAAATGCCAGTAGAGCAA |
| | 117del Probe | |
| 101 | THS for tcdC | CAATAAATGCCAGTAGAGCAA |
| | 117del Probe | |
| 102 | THS for tcdC | CATAAATGCCAGTAGAGCAA |
| | 117del Probe | |
| 103 | THS for tcdC | CAAAAUAAATGCCAGTAGAGCAA |
| | 117del Probe | |
| | w/methoxy | |
| 104 | THS for tcdC | CAAmAATAAATGCCAGTAGAGCAA |
| | 117del Probe | |
| | w/methoxy | |
| 105 | THS for tcdC | CAmAAATAAATGCCAGTAGAGCAA |
| | 117del Probe | |
| | w/methoxy | |
| 106 | THS for tcdC | CAAAUAAATGCCAGTAGAGCAA |
| | 117del Probe | |
| | w/methoxy | |
| 107 | THS for tcdC | CAAUAAATGCCAGTAGAGCAA |
| | 117del Probe | |
| | w/methoxy | |
| 108 | THS for tcdC | CAUAAATGCCAGTAGAGCAA |
| | 117del Probe | |
| | w/methoxy | |
| 109 | THS for tcdC | CAAmAUAAATGCCAGTAGAGCAA |
| | 117del Probe | |
| | w/methoxy | |
| 110 | THS for tcdC | CAAAUmAAATGCCAGTAGAGCAA |
| | 117del Probe | |
| | w/methoxy | |
| 111 | THS for tcdC | CAAmAUmAAATGCCAGTAGAGCAA |
| | 117del Probe | |
| | w/methoxy | |
| 112 | THS for tcdC | CmAAATAAATGCCAGTAGAGCAA |
| | 117del Probe | |
| | w/methoxy | |
| 113 | THS for tcdC | CmAAAATAAATGCCAGTAGAGCAA |
| | 117del Probe | |
| | w/methoxy | |
| 114 | THS for tcdC | CmAAAUAAATGCCAGTAGAGCAA |
| | 117del Probe | |
| | w/methoxy | |
| 115 | THS for tcdC | CmAAAAUAAATGCCAGTAGAGCAA |
| | 117del Probe | |
| | w/methoxy | |
| 116 | THS for tcdC | CAAAUmAAATGCCAGTAGAGCAATA |
| | 117del Probe | |
| | w/methoxy | |
| 117 | THS for tcdC | CAAAUmAAATGCCAGTAGAGCAATAC |
| | 117del Probe | |
| | w/methoxy | |
| 118 | THS for tcdC | CAAAUmAAATGCCAGTAGAGCAATACC |
| | 117del Probe | |
| | w/methoxy | |
| 119 | THS for tcdC | CAAAUmAAATGCCAGTAGAGCAATACCC |
| | 117del Probe | |
| | w/methoxy | |
| 120 | THS for tcdC | CAAAUmAAATGmCmCAGTAGAGmCAATA |
| | 117del Probe | |
| | w/methoxy | |
| 121 | THS for tcdC | mCAAAUmAAATGmCmCAGTAGAGmCAATA |
| | 117del Probe | |
| | w/methoxy | |
| 122 | THS for tcdC | mCAAAUmAAATGCCAGTAGAGCAATA |
| | 117del Probe | |
| | w/methoxy | |
| 123 | THS for tcdC | CAAAATAAATGCCAGTAGAGC |
| | 117del Probe | |
| 124 | THS for tcdC | CAAAATAAATGCCAGTAGAGC |
| | 117del Probe | |
| 125 | THS for tcdC | CAAAATAAATGCCAGTAGAG |
| | 117del Probe | |
| 126 | THS for tcdC | CAAAATAAATGCCAGTAGA |
| | 117del Probe | |
| 127 | THS for tcdC | AAAATAAATGCCAGTAGAGCAATAC |
| | 117del Probe | |
| 128 | THS for tcdC | AAAATAAATGCCAGTAGAGCAATAT |
| | 117del Probe | |
| 129 | THS for tcdC | AAAATAAATGCCAGTAGAGCAATATC |
| | 117del Probe | |
| 130 | THS for tcdC | C(abasic)AAATAAATGCCAGTAGAGCAA |
| | 117del Probe with | |
| | abasic linker | |
| 131 | THS for tcdC | CA(abasic)AATAAATGCCAGTAGAGCAA |
| | 117del Probe with | |
| | abasic linker | |
| 132 | THS for tcdC | C(sp9)AAATAAATGCCAGTAGAGCAA |
| | 117del Probe with | |
| | abasic linker | |
| 133 | THS for tcdC | CA(sp9)AATAAATGCCAGTAGAGCAA |
| | 117del Probe with | |
| | abasic linker | |
| 134 | THS for tcdC | C(abasic)AAATAAATGCCAGTAGAGC |
| | 117del Probe with | |
| | abasic linker | |
| 135 | THS for tcdC | CA(abasic)AATAAATGCCAGTAGAGC |
| | 117del Probe with | |
| | abasic linker | |
| 136 | THS for tcdC | C(sp9)AAATAAATGCCAGTAGAGC |
| | 117del Probe with | |
| | abasic linker | |
| 137 | THS for tcdC | CA(sp9)AATAAATGCCAGTAGAGC |
| | 117del Probe with | |
| | abasic linker | |
| 138 | tcdC 117del Primary | cgcgccgaggCAAAATAAATGCCAGTAGAGC-hexanediol |
| | Probe | |
| 139 | tcdC 117del Primary | cgcgccgaagAAAATAAATGCCAGTAGAGCAATAT-hexanediol |
| | Probe | |
| 140 | tcdC 117del Primary | cgcgccgaggAAAATAAATGCCAGTAGAGCAATATC-hexanediol |
| | Probe | |
| 141 | tcdC 117del Primary | cgcgccgaggCAAAATAAATGCCAGTAGAG-hexanediol |
| | Probe | |
| 142 | tcdC 117del Primary | cgcgccgaggCAAAATAAATGCCAGTAGA-hexanediol |
| | Probe | |
| 143 | tcdC 117del Primary | cgcgccgaggCAAAATAAATGCCAGTAGAGCAA-hexanediol |
| | Probe | |
| 144 | tcdC 117del Primary | cgcgccgaggCAAAATAAATGCCAGTAGAGCAATAC-hexanediol |
| | Probe | |
| 145 | tcdC 117del Primary | cgcgccgaggCAAATAAATGCCAGTAGAGCAA-hexanediol |
| | Probe | |
| 146 | tcdC 117del Primary | cgcgccgaggCAATAAATGCCAGTAGAGCAA-hexanediol |
| | Probe | |
| 147 | tcdC 117del Primary | cgcgccgaggCATAAATGCCAGTAGAGCAA-hexanediol |
| | Probe | |
| 148 | tcdC 117del Primary | cgcgccgaggCAAAAUAAATGCCAGTAGAGCAA-hexanediol |
| | Probe w/methoxy | |
| 149 | tcdC 117del Primary | cgcgccgaggCAAmAATAAATGCCAGTAGAGCAA-hexanediol |
| | Probe w/methoxy | |
| 150 | tcdC 117del Primary | cgcgccgaggCAmAAATAAATGCCAGTAGAGCAA-hexanediol |
| | Probe w/methoxy | |
| 151 | tcdC 117del Primary | cgcgccgaggCAAAUAAATGCCAGTAGAGCAA-hexanediol |
| | Probe w/methoxy | |
| 152 | tcdC 117del Primary | cgcgccgaggCAAUAAATGCCAGTAGAGCAA-hexanediol |
| | Probe w/methoxy | |
| 153 | tcdC 117del Primary | cgcgccgaggCAUAAATGCCAGTAGAGCAA-hexanediol |
| | Probe w/methoxy | |
| 154 | tcdC 117del Primary | cgcgccgaggCAAmAUAAATGCCAGTAGAGCAA-hexanediol |
| | Probe w/methoxy | |
| 155 | tcdC 117del Primary | cgcgccgaggCAAmAUmAAATGCCAGTAGAGCAA-hexanediol |
| | Probe w/methoxy | |
| 156 | tcdC 117del Primary | cgcgccgaggCAAmAUmAAATGCCAGTAGAGCAA-hexanediol |
| | Probe w/methoxy | |
| 157 | tcdC 117del Primary | cgcgccgaggCmAAATAAATGCCAGTAGAGCAA-hexanediol |
| | Probe w/methoxy | |
| 158 | tcdC 117del Primary | cgcgccgaggCmAAAATAAATGCCAGTAGAGCAA-hexanediol |
| | Probe w/methoxy | |
| 159 | tcdC 117del Primary | cgcgccgaggCmAAAUAAATGCCAGTAGAGCAA-hexanediol |
| | Probe w/methoxy | |
| 160 | tcdC 117del Primary | cgcgccgaggCmAAAAUAAATGCCAGTAGAGCAA-hexanediol |
| | Probe w/methoxy | |
| 161 | tcdC 117del Primary | cgcgccgaggCAAAUmAAATGCCAGTAGAGCAATA-hexanediol |
| | Probe w/methoxy | |
| 162 | tcdC 117del Primary | cgcgccgaggCAAAUmAAATGCCAGTAGAGCAATAC-hexanediol |
| | Probe w/methoxy | |
| 163 | tcdC 117del Primary | cgcgccgaggCAAAUmAAATGCCAGTAGAGCAATACC-hexanediol |
| | Probe w/methoxy | |
| 164 | tcdC 117del Primary | cgcgccgaggCAAAUmAAATGCCAGTAGAGCAATACCC-hexanediol |
| | Probe w/methoxy | |
| 165 | tcdC 117del Primary | cgcgccgaggCAAAUmAAATGmCmCAGTAGAGmCAATA-hexanediol |
| | Probe w/methoxy | |
| 166 | tcdC 117del Primary | cgcgccgaggmCAAAUmAAATGmCmCAGTAGAGmCAATA-hexanediol |
| | Probe w/methoxy | |
| 167 | tcdC 117del Primary | cgcgccgaggmCAAAUmAAATGCCAGTAGAGCAATA-hexanediol |
| | Probe w/methoxy | |
| 168 | tcdC 117del Primary | atgacgtggcagacCAAAATAAATGCCAGTAGAGC-hexanediol |
| | Probe | |
| 169 | tcdC 117del Primary | acggacgcggagCAAAATAAATGCCAGTAGAGC-hexanediol |
| | Probe | |
| 170 | tcdC 117del Primary | acggacgcggagCAAAATAAATGCCAGTAGAG-hexanediol |
| | Probe | |
| 171 | tcdC 117del Primary | acggacgcggagCAAAATAAATGCCAGTAGA-hexanediol |
| | Probe | |
| 172 | tcdC 117del Primary | acggacgcggagAAAATAAATGCCAGTAGAGCAATAC-hexanediol |
| | Probe | |
| 173 | tcdC 117del Primary | acggacgcggagAAAATAAATCCAGTAGAGCAATAT-hexanediol |
| | Probe | |
| 174 | tcdC 117del Primary | acggacgcggagAAAATAAATGCCAGTAGAGCAATATC-hexanediol |
| | Probe | |
| 175 | tcdC 117del Primary | acggacgcggagC(abasic)AAATAAATGCCAGTAGAGCAA-hexanediol |
| | Probe with abasic | |
| | linker | |
| 176 | tcdC 117del Primary | acggacgcggagCA(abasic)AATAAATGCCAGTAGAGCAA-hexanediol |
| | Probe with abasic | |
| | linker | |
| 177 | tcdC 117del Primary | acggacgcggagcC(sp9)AAATAAATGCCAGTAGAGCAA-hexanediol |
| | Probe with C9 linker | |
| 178 | tcdC 117del Primary | acggacgcggagCA(sp9)AATAAATGCCAGTAGAGCAA-hexanediol |
| | Probe with C9 linker | |
| 179 | tcdC 117del Primary | acggacgcggagC(abasic)AAATAAATGCCAGTAGAGC-hexanediol |
| | Probe with abasic | |
| | linker | |
| 180 | tcdC 117del Primary | acggacgcggagCA(abasic)AATAAATGCCAGTAGAGC-hexanediol |
| | Probe with abasic | |
| | linker | |
| 181 | tcdC 117del Primary | acggacgcggagC(sp9)AAATAAATGCCAGTAGAGC-hexanediol |
| | Probe with C9 linker | |
| 182 | tcdC 117del Primary | acggacgcggagCA(sp9)AATAAATGCCAGTAGAGC-hexanediol |
| | Probe with C9 linker | |
| 183 | tcdC 117del Invader | ACAAAGGGTATTGCTCTACTGGCATTTATTTc |
| 184 | THS for tcdC | TGGtGTGTTTTTTGGCAATAT |
| | 117del Primary | |
| | Probe | |
| 185 | THS for tcdC | GGtGTGTTTTTTGGCAATATATC |
| | 117del Primary | |
| | Probe | |
| 186 | THS for tcdC | GGtGTGTTTTTTGGCAATAT |
| | 117del Primary | |
| | Probe | |
| 187 | THS for tcdC | GGtGTGTTTTTTGGCAATATAC |
| | 117del Primary | |
| | Probe | |
| 188 | tcdC 117del Primary | acggacgcggagTGGtGTGTTTTTTGGCAATAT-hexanediol |
| | Probe | |
| 189 | tcdC 117del Primary | atgacgtggcagacGGtGTGTTTTTTGGCAATATAC-hexanediol |
| | Probe | |
| 190 | tcdC 117del Primary | atgacgtggcagacGGtGTGTTTTTTGGCAATAT-hexanediol |
| | Probe | |
| 191 | tcdC 117del Primary | acggacgcggagGGtGTGTTTTTTGGCAATATATC-hexanediol |
| | Probe | |
| 192 | tcdC 117del Reverse | GGTGAGGATATATTGCCAAAAAACACGCC |
| | iPrimer | |
| 193 | tcdC 117del Reverse | GGTGAGGATATATTGCCAAAAAACACGCCA |
| | iPrimer | |
| 194 | tcdC 183/184 | CTGAAGACCATGAGGAGGTCATTTCTAATa |
| | Invader | |
| 195 | tcdC 183/184 | GTTCTGAAGACCATGAGGAGGTCATTTCTAAG |
| | Invader | |
| 196 | tcdC 183/184 | GTTCTGAAGACCATGAGGAGGTCATTTCTAATG |
| | Invader | |
| 197 | THS for tcdC | TTAAACATCAGTTATAGATTCTC |
| | 184/184 CC > TT | |
| | Primary Probe | |
| 198 | THS for tcdC | TAAACATCAGTTATAGATTCTC |
| | 184/184 CC > TT | |
| | Primary Probe | |
| 199 | THS for tcdC | TTAAACATCAGTTATAGATTCTC |
| | 184/184 CC > TT | |
| | Primary Probe | |
| 200 | THS for tcdC | TAAAmCATCAGTTATAGATTCTC |
| | 184/184 CC > TT | |
| | Primary Probe | |
| 201 | tcdC 184/184 | cgcgccgaggTTAAACATCAGTTATAGATTCTC-hexanediol |
| | CC > TT | |
| | Primary Probe | |
| 202 | tcdC 184/184 | cgcgccgaggTAAACATCAGTTATAGATTCTC-hexanediol |
| | CC > TT | |
| | Primary Probe | |
| 203 | tcdC 184/184 | cgcgccgaggTTAAACATCAGTTATAGATTCTC-hexanediol |
| | CC > TT | |
| | Primary Probe | |
| 204 | tcdC 184/184 | cgcgccgaggTAAAmCATCAGTTATAGATTCTC-hexanediol |
| | CC > TT | |
| | Primary Probe | |
| 205 | tcdC 184 Invader | GTTTCTATTTCTGTTTTTTGAGAATCTATAACTGATGTTTt |
| 206 | THS for tcdC | AATTAGAAATGACCTCCTCATGG |
| | 183C > T 184C > T | |
| | Primary Probe | |
| 207 | THS for tcdC | ATTAGAAATGACCTCCTCATGGT |
| | 183C > T 184C > T | |
| | Primary Probe | |
| 208 | THS for tcdC | AATTAGAAATGACCTCCTC |
| | 183C > T 184C > T | |
| | Primary Probe | |
| 209 | THS for tcdC | ATTAGAAATGACCTCCTC |
| | 183C > T 184C > T | |
| | Primary Probe | |
| 210 | THS for tcdC | AATTAGAAATGACCTCCTCATGG |
| | 183C > T 184C > T | |
| | Primary Probe | |
| 211 | THS for tcdC | AATTAGAAATGACCTCCTCATGG |
| | 183C > T 184C > T | |
| | Primary Probe | |
| 212 | THS for tcdC | ATTAGAAATGACCTCCTCATGGT |
| | 183C > T 184C > T | |
| | Primary Probe | |
| 213 | tcdC 183C > T | cgcgccgaggAATTAGAAATGACCTCCTCATGG-hexanediol |
| | 184C > T | |
| | Primary Probe | |
| 214 | tcdC 183C > T | cgcgccgaggATTAGAAATGACCTCCTCATGGT-hexanediol |
| | 184C > T | |
| | Primary Probe | |
| 215 | tcdC 183C > T | cgcgccgaggAATTAGAAATGACCTCCTC-hexanediol |
| | 184C > T | |
| | Primary Probe | |
| 216 | tcdC 183C > T | cgcgccgaggATTAGAAATGACCTCCTC-hexanediol |
| | 184C > T | |
| | Primary Probe | |
| 217 | tcdC 183C > T | atgacgtggcagacAATTAGAAATGACCTCCTCATGG-hexanediol |
| | 184C > T | |
| | Primary Probe | |
| 218 | tcdC 183C > T | acggacgcggagAATTAGAAATGACCTCCTCATGG-hexanediol |
| | 184C > T | |
| | Primary Probe | |
| 219 | tcdC 183C > T | acggacgcggagATTAGAAATGACCTCCTCATGGT-hexanediol |
| | 184C > T | |
| | Primary Probe | |
| 220 | tcdC 183T Forward | CTTGTTCTGAAGACCATGAGGAGGTCATTTCTAAT |
| | iPrimer (+) | |
| 221 | tcdC 184 Forward | GTTTCTATTTCTGTTTTTTTGAGAATCTATAACTGATGTTTAA |
| | iPrimer (−) | |
| 222 | tcdC 184 Forward | GCAATATATCCTCACCAGCTTGTTCTGAAG |
| | Primer | |
| 223 | tcdC 184 Invader | GAAGACCATGAGGAGGTCATTTCTAACa |
| 224 | Not Used | Not Used |
| 225 | tcdC 184 Iprimer 2 | GTTTCTATTTCTGTTTTTTGAGAATCTATAACTGATGTTTA |
| | (−) | |
| 226 | Not Used | |
| 227 | Not Used | |
| 228 | Not Used | |
| 229 | Not Used | |
| 230 | Not Used | |
| 231 | Not Used | |
| 232 | Not Used | |
| 233 | THS for tcdC | AGTTAGAAATGACCTCCTCATG |
| | 184C > T Primary | |
| | Probe | |
| 234 | THS for tcdC | TAAACATCAGTTATAGATTCTCAAAAAGC |
| | 184C > T Primary | |
| | Probe | |
| 235 | THS for tcdC | TAAACATCAGTTATAGATTCTCAAAAAGC |
| | 184C > T Primary | |
| | Probe | |
| 236 | THS for tcdC | AGTTAGAAATGACCTCCTCATG |
| | 184C > T Primary | |
| | Probe | |
| 237 | THS for tcdC | TAAACATCAGTTATAGATTCTC |
| | 184C > T Primary | |
| | Probe | |
| 238 | tcdC 184C > T | atgacgtggcagacAGTTAGAAATGACCTCCTCATG-hexanediol |
| | Primary Probe | |
| 239 | tcdC 184C > T | atgacgtggcagacTAAACATCAGTTATAGATTCTCAAAAAGC-hexanediol |
| | Primary Probe | |
| 240 | tcdC 184C > T | acggacgcggagTAAACATCAGTTATAGATTCTCAAAAAGC-hexanediol |
| | Primary Probe | |
| 241 | tcdC 184C > T | acggacgcggagAGTTAGAAATGACCTCCTCATG-hexanediol |
| | Primary Probe | |
| 242 | tcdC 184C > T | acggacgcggagTAAACATCAGTTATAGATTCTC-hexanediol |
| | Primary Probe | |
| 243 | tcdC Forward Primer | GGAAAAGGAAGCTCTAAGAAAATAATTAAATTCTTTAAGAGC |
| 244 | tcdC Forward Primer | GGAAGCTCTAAGAAAATAATTAAATTCTTTAAGAGCACAAAGGG |
| 245 | tcdC Forward Primer | GGTAAmCGAATTTAGTAATGAA |
| 246 | tcdC Forward Primer | GGTAAmCGAATTTAGTAATGAA |
| 247 | tcdC Forward Primer | GGAAGCTCTAAGAAAATAATTAAATTCTTTAAGAGCACAAAGG |
| 248 | tcdC Forward Primer | GGTAACGAATTTAGTAATGAAGGAAAAGGAAGC |
| 249 | tcdC Reverse Primer | GTTCAGCATCAGACAATTTGCTATTTAAAGTTTCTATTTC |
| 250 | tcdC Reverse Primer | GAACCATGGTTCAGCATCAGACAATTTGC |
| 251 | tcdC Reverse Primer | CTTTCTTTTCGTCGTCTTTCATTTTGAACCATGG |
| 252 | tcdC Reverse Primer | CAATAGCTTTCTTTTCGTCGTCTTTCATTTTGAACC |
| 253 | tcdC Reverse Primer | TCATAAGTAATACCAGTATCATATC |
| 254 | tcdC Reverse Primer | TCATAAGTAATACCAGTATCATATCC |
| 255 | tcdC Reverse Primer | GTCGTCTTTCATTTTGAACCATGGTTCAGC |
| 256 | tcdC Reverse Primer | GTTTCTATTTCTGTTTTTTGAGAATCTATAACTGATGTTTGA |
| 257 | tcdC Reverse Primer | CCTCCTCATGGTCTTCAGAACAAG |
| 258 | tcdC Reverse Primer | CCTCCTCATGGTCTTCAGAACAAGC |
| 259 | tcdC Reverse Primer | GACCTCCTCATGGTCTTCAGAACAAG |

tcdB oligomers

| | | |
|---|---|---|
| 260 | tcdB Forward iPrimer | GTAAGTTTAGGTGCAGCAATCAAAGAGCT |
| 261 | tcdB Forward iPrimer | GTAAGTTTAGGTGCAGCAATCAAAGAGTT |
| 262 | tcdB Forward iPrimer | GGATTACCTGTAATTGCTACTATTATAGATGGTGTAAGT |
| 263 | tcdB Forward iPrimer | GATTACCTGTAATTGCTACTATTATAGATGGTGTAAGT |
| 264 | tcdB Forward Primer | GTGTAAGTTTAGGTGCAGCAATCAAAGAG |
| 265 | tcdB Forward Primer | GGTGCAGCAATCAAAGAG |
| 266 | tcdB Forward Primer | TGGTGTAAGTTTAGGTGCAGCAATCAAAGAG |
| 267 | tcdB Forward Primer | GATTGGAGGGTCAAATAAATGACACTGCTATTAAT |
| 268 | tcdB Forward Primer | ATAGATGGTGTAAGTTTAGGTGCAGCAATC |
| 269 | tcdB Forward Primer | TGGTGTAAGTTTAGGTGCATCAATTAAAGAG |
| 270 | tcdB Forward Primer | GGTGCAGCAATCAAAGAGCTAAGTG |
| 271 | tcdB Forward Primer | GTAAGTTTAGGTGCATCAATTAAAGAGTTGAGTG |
| 272 | tcdB Forward Primer | GTGTAAGTTTAGGTGCATCAATTAAAGAGTTGAGTG |
| 273 | tcdB Forward Primer | GTGGATTTAGTATACTTTTAGTTC |
| 274 | tcdB Forward Primer | GTATTACCTAATGCTCCAAATAGAGTATTTGCTTG |
| 275 | tcdB Forward Primer | CCTAATGCCCAGATAGAGTATTTGGCTG |
| 276 | tcdB Forward Primer | GCCCCAGATAGAATCTTTGGCTG |
| 277 | tcdB Forward Primer | CTAATGCCCCAGATAGAATCTTTGGCTG |
| 278 | tcdB Forward Primer | CTTGATTTGTCAAAAGATTTAATG |
| 279 | tcdB Forward Primer | mCTTGATTTATmCAAAAGATTTAATG |
| 280 | tcdB Invader | TTTACTGCCATTATACCTATCTTAGCTTCTATTTa |
| 281 | THS for tcdB | CTTGTCTTAATAATGGGTCACT |
| | Primary Probe | |
| 282 | THS for tcdB | TAATTATATAAATCAATGGAAAGATGTAAATAGTG |
| | Primary Probe | |
| 283 | THS for tcdB | GGGAAAGAGGATGGACG |
| | Primary Probe | |
| 284 | THS for tcdB | GGGAAAGAGGATGGACGC |
| | Primary Probe | |
| 285 | THS for tcdB | GGGAAAGAGGATGGAC |
| | Primary Probe | |
| 286 | THS for tcdB | GGGAAAGAGGATGGACGCCAG |
| | Primary Probe | |
| 287 | THS for tcdB | GGGAAACAGGATGGACACC |
| | Primary Probe | |
| 288 | THS for tcdB | GGGAAAGAGGATGGACGCC |
| | Primary Probe | |
| 289 | THS for tcdB | ATTATAGTCACTATTTACATCTTTCCATTGATTTATAT |
| | Primary Probe | |
| 290 | THS for tcdB | CAAAGAGCTAAGTGAAACCAGTGAC |
| | Primary Probe | |
| 291 | THS for tcdB | CAAAGAGCTAAGTGAAACCAGTGACC |
| | Primary Probe | |
| 292 | THS for tcdB | CAAAGAGCTAAGTGAAACCAGTGACCC |
| | Primary Probe | |
| 293 | THS for tcdB | TAAGTGAAACCAGTGACCCATTATTAAGAC |
| | Primary Probe | |
| 294 | THS for tcdB | CTTGTCTTAATAATGGGTCACT |
| | Primary Probe | |
| 295 | THS for tcdB | TGAGTGAAACCAGTGACCCATTATTAAGAC |
| | Primary Probe | |
| 296 | THS for tcdB | TTTAGGTGCATCAATTAAAGAGTTG |
| | Primary Probe | |
| 297 | THS for tcdB | TTTAGGTGCAGCAATTAAAGAGTTA |
| | Primary Probe | |
| 298 | THS for tcdB | CTTGTmCTTAATAATGGGTmCAmCT |
| | Primary Probe | |
| 299 | tcdB Primary Probe | cgcgccgaggCTTGTCTTAATAATGGGTCACT-hexanediol |
| 300 | tcdB Primary Probe | acggacgcggagTAATTATATAAATCAATGGAAAGATGTAAATAGTG-hexanediol |
| 301 | tcdB Primary Probe | acggacgcggagGGGAAAGAGGATGGACG-hexanediol |
| 302 | tcdB Primary Probe | acggacgcggagGGGAAAGAGGATGGACGC-hexanediol |
| 303 | tcdB Primary Probe | acggacgcggagGGGAAAGAGGATGGAC-hexanediol |
| 304 | tcdB Primary Probe | acggacgcggagGGGAAAGAGGATGGACGCCAG-hexanediol |
| 305 | tcdB Primary Probe | acggacgcggagGGGAAACAGGATGGACACC-hexanediol |
| 306 | tcdB Primary Probe | acggacgcggagGGGAAAGAGGATGGACGCC-hexanediol |
| 307 | tcdB Primary Probe | acggacgcggagATTATAGTCACTATTTACATCTTTCCATTGATTTATAT- |
| | | hexanediol |
| 308 | tcdB Primary Probe | ACGGACGCGGAGCAAAGAGCTAAGTGAAACCAGTGAC-hexanediol |
| 309 | tcdB Primary Probe | ACGGACGCGGAGCAAAGAGCTAAGTGAAACCAGTGACC-hexanediol |
| 310 | tcdB Primary Probe | ACGGACGCGGAGCAAAGAGCTAAGTGAAACCAGTGACCC-hexanediol |
| 311 | tcdB Primary Probe | ACGGACGCGGAGTAAGTGAAACCAGTGACCCATTATTAAGAC-hexanediol |
| 312 | tcdB Primary Probe | acggacgcggagCTTGTCTTAATAATGGGTCACT-hexanediol |
| 313 | tcdB Primary Probe | ACGGACGCGGAGTGAGTGAAACCAGTGACCCATTATTAAGAC-hexanediol |
| 314 | tcdB Primary Probe | acggacgcggagTTTAGGTGCATCAATTAAAGAGTTG-hexanediol |
| 315 | tcdB Primary Probe | acggacgcggagTTTAGGTGCAGCAATTAAAGAGTTA-hexanediol |
| 316 | tcdB Primary Probe | acggacgcggagCTTGTmCTTAATAATGGGTmCAmCT-hexanediol |
| 317 | tcdB Reverse iPrimer | TTTACTGCCATTATACCTATCTTAGCTTCTATTTC |
| 318 | tcdB Reverse Primer | CTGCCATTATACCTATCTTAGCTTCTATTTCTTGTC |
| 319 | tcdB Reverse Primer | CTGCCATTATACCTATCTTAGCTTCTATTTCTTG |
| 320 | tcdB Reverse Primer | CTGCCATTATACCTATTTTTGCTTCTATTTCTTG |
| 321 | tcdB Forward Primer | GTAAGTTTAGGTGCATCAATTAAAGAGTT |
| 322 | tcdB Reverse Primer | GAACTAAAAGTATACTAAATCCACTAGC |
| 323 | tcdB Reverse iPrimer | TCAATGTGTTTATCAAAAATGCATTACTATCATAAAAAACATTAACA |
| 324 | tcdB Reverse iPrimer | TTTACTGCCATTATACCTATTTTTGCTTCTATTTC |
| 325 | tcdB Reverse iPrimer | TTTACTGCCATTATACCTATTTTGGCTTCTATTTC |
| 326 | tcdB Reverse iPrimer | GTTTACTGCCATTATACCTATTTTGGCTTCTATTTC |
| 327 | tcdB Reverse Primer | CAATGTGTTTATCAAAAATGCATTACTATCATAAAAAACATTAACAT |
| 328 | tcdB Reverse Primer | CTATTTCTTGTCTTAATAATGGGTCACTTGTTTCAC |
| 329 | tcdB Reverse Primer | CTTGTCTTAATAATGGGTCACTTGTTTCAC |
| 330 | tcdB Reverse Primer | CTATTTCTTGTCTTAATAATGGGTCACTAGTTTCAC |
| 331 | tcdB Reverse Primer | CTTGTCTTAATAATGGGTCACTAGTTTCAC |
| 332 | tcdB Reverse Primer | CCTGCTGAAATTCCTGCTAAAGGAACTAAAAGTATACTAAATCC |
| 333 | tcdB Reverse Primer | CCTGCTGAAATTCCTGCTAAAGGAACTAAAAGTATACTAAATC |
| 334 | tcdB Reverse Primer | ACCTGCTGAAATTCCTGCTAAAGGAACTAAAAGTATAC |
| 335 | tcdB Reverse Primer | AGTTTTGTACCATCATTTTCTAAGCTTCTTAAACC |
| 336 | tcdB Reverse Primer | CAGTTTTGTGCCATCATTTTCTAAGCTTCTTAAACC |
| 337 | tcdB Reverse Primer | CTCTTATACGGTCTAACAG |
| 338 | tcdB Reverse Primer | mCTCTTATAmCGGTCTAATAG |
| 339 | tcdB Forward Primer | TGGATTGGAGGTCAAATAAATGATACTGCTATT |

¹lowercase bases have RNA backbone; uppercase bases have DNA backbone; mC indicates a 5-methylated
cytosine; 2′-O-Methyladenosine; Hdiol = Hexanediol, HEX = Hexochloro-Fluorescein, FAM = Fluoresecin,
Red = Redmond Red fluorphore, BBQdT = Blackberry Quencher 650 dT, Ecl = Eclipse Quencher. C9 (also
referred to as sp9) indicates spacer 9 linker (triethylene glycol chain).

1. A composition or kit comprising forward and reverse tcdC amplification oligomers and further comprising one or both of a first and a second tcdC detection oligomer, wherein:
the first tcdC detection oligomer, if present, is configured to specifically hybridize to a first tcdC sequence at a site comprising position 117 thereof, but exhibits distinguishably different hybridization to a second tcdC sequence, wherein the first tcdC sequence is the sequence of SEQ ID NO: 3 and the second tcdC sequence is the sequence of SEQ ID NO: 4;
the second tcdC detection oligomer, if present, is configured to specifically hybridize to the sequence of a third tcdC sequence at a site comprising position 184 thereof, but exhibits distinguishably different hybridization to the sequence of a fourth tcdC sequence, wherein the third tcdC sequence is the sequence of SEQ ID NO: 5 and the fourth tcdC sequence is the sequence of SEQ ID NO: 2; and
the forward and reverse tcdC amplification oligomers are configured to produce a tcdC amplicon, wherein the tcdC amplicon comprises position 117 of SEQ ID NO: 3 or SEQ ID NO: 4 if the first tcdC detection oligomer is present and/or position 184 of SEQ ID NO: 5 if the second tcdC detection oligomer is present.

2. (canceled)

3. A method of detecting a C. difficile tcdC allele, the C. difficile tcdC allele comprising a 117del mutation or a 184T mutation, the method comprising:
preparing a composition according to claim 1 which further comprises a sample comprising or suspected of comprising C. difficile nucleic acid;
subjecting the composition to amplification conditions; and
detecting the presence of the C. difficile 117del tcdC allele or 184T tcdC allele in the sample by determining whether at least one of the first tcdC detection oligomer or the second tcdC detection oligomer hybridized to a tcdC amplicon.

4-10. (canceled)

11. The composition or kit of claim 1, wherein the first tcdC detection oligomer is present and competes for hybridization to a tcdC nucleic acid under stringent conditions with an oligomer having a sequence consisting of SEQ ID NO: 86-92, 183, 192, or 193.

12-16. (canceled)

17. The composition or kit of claim 11, wherein the first tcdC detection oligomer is present and has no mismatches to the 1, 2, 3, 4, 5, 6, 7, 8, 9, or 10 3′-terminal nucleotides of SEQ ID NO: 86-92, 183, 192, or 193.

18. The composition or kit of claim 17, wherein the first tcdC detection oligomer is present and comprises the sequence of of SEQ ID NO: 86 or 183.

19. (canceled)

20. The composition or kit of claim 1, wherein:
(i) the first tcdC detection oligomer is present and comprises the sequence any one of SEQ ID NOs: 89-92; and
(ii) the composition or kit further comprises an additional first tcdC detection oligomer that comprises the sequence any one of SEQ ID NOs: 89-92, which is different from the first tcdC detection oligomer.

21. The composition or kit of claim 1, wherein the composition or kit further comprises at least one first tcdC primary probe oligomer that is configured to form an invasive cleavage structure in the presence of the first tcdC detection oligomer and a polynucleotide comprising the sequence of SEQ ID NO: 3.

22-27. (canceled)

28. The composition or kit of claim 21, wherein the first tcdC primary probe oligomer comprises the sequence of any one of SEQ ID NOs: 93-137 or 184-187.

29-31. (canceled)

32. The composition or kit of claim 1, wherein the second tcdC detection oligomer is present.

33. The composition or kit of claim 32, wherein the second tcdC detection oligomer comprises the sequence of any one of SEQ ID NOs: 194-196 or 205, with up to two mismatches.

34-35. (canceled)

36. The composition or kit of claim 33, wherein the second tcdC detection oligomer has no mismatches to the 1, 2, 3, 4, 5, 6, 7, 8, 9, or 10 3′-terminal nucleotides of SEQ ID NO: 194-196 or 205.

37. The composition or kit of claim 36, wherein the second tcdC detection oligomer comprises the sequence of any one of SEQ ID NOs: 194-196 or 205.

38. The composition or kit of claim 1, wherein the composition or kit further comprises at least one second tcdC primary probe oligomer that is configured to form an invasive cleavage structure in the presence of the second tcdC detection oligomer and a polynucleotide comprising the sequence of SEQ ID NO: 5.

39-56. (canceled)

57. The composition or kit of claim 1, wherein the forward tcdC amplification oligomer comprises the sequence of any one of SEQ ID NOs: 243-248.

58-62. (canceled)

63. The composition or kit of claim 1, wherein the reverse tcdC amplification oligomer comprises the sequence of any one of SEQ ID NOs: 249-259.

64. The composition or kit of claim 1, wherein the composition or kit further comprises at least forward and reverse tcdA amplification oligomers, and at least one tcdA detection oligomer, wherein:
the forward and reverse tcdA amplification oligomers are configured to produce a tcdA amplicon having a size of from 80 to 400 nucleotides; and
the tcdA detection oligomer is configured to specifically hybridize to the tcdA amplicon.

65-72. (canceled)

73. The composition or kit of claim 64, wherein the forward tcdA amplification oligomer comprises the sequence of any one of SEQ ID NOs: 59-60, 64, or 65.

74-88. (canceled)

89. The composition or kit of claim 1, wherein the composition or kit further comprises forward and reverse tcdB amplification oligomers, wherein:
the forward and reverse tcdB amplification oligomers are configured to produce a tcdB amplicon having a size of from 80 to 400 nucleotides; and
the tcdB detection oligomer is configured to specifically hybridize to the tcdB amplicon.

90-123. (canceled)

124. The composition or kit of claim 1, wherein the composition or kit further comprises forward and reverse cdtB amplification oligomers, and at least one cdtB detection oligomer, wherein:
the forward and reverse cdtB amplification oligomers are configured to produce a cdtB amplicon having a size of from 80 to 400 nucleotides; and
the cdtB detection oligomer is configured to specifically hybridize to the cdtB amplicon.

125-150. (canceled)

151. The composition or kit of claim 1, wherein the composition or kit further comprises forward and reverse cdtA amplification oligomers, and at least one cdtA detection oligomer, wherein:
the forward and reverse cdtA amplification oligomers are configured to produce a cdtA amplicon having a size of from 80 to 400 nucleotides; and
the cdtA detection oligomer is configured to specifically hybridize to the cdtA amplicon.

152-211. (canceled)


SEQUENCE LISTING (339 sequences)

SEQ ID NO: 1 - DNA, 681 bp, Clostridium difficile
  source 1..681: mol_type = genomic DNA
atgttttctaaaaaaaatgagggtaacgaatttagtaatgaaggaaaaggaagctctaagaaaataattaaattctttaagagcacaaagggtattgctctactggcatttattttgggcgtgttttttggcaatatatcctcaccagcttgttctgaagaccatgaggaggtcatttctaatcaaacatcagttatagattctcaaaaaacagaaatagaaactttaaatagcaaattgtctgatgctgaaccatggttcaaaatgaaagacgacgaaaagaaagctattgaagctgaaaatcaacgtaaagctgaagaagctaaaaaggctgaagaacaacgtaaaaaagaagaagaagagaagaaaggatatgatactggtattacttatgaccaattagctagaacacctgatgattataagtacaaaaaggtaaaatttgaaggtaaggttattcaagttattgaagatggtgatgaggtgcaaataagattagctgtgtctggaaattatgataaggtcgtactatgtagttataaaaaatcaataactccttcaagagtgttagaggatgattacataactataagaggtataagtgctggaactataacttatgaatcaactatgggtggaaatataactataccagggatagctgtagagaaaataaattaa

SEQ ID NO: 2 - DNA, 480 bp, Clostridium difficile
  source 1..480: mol_type = genomic DNA
atgttttctaaaaaaaatgagggtaacgaatttagtaatgaaggaaaaggaagctctaagaaaataattaaattctttaagagcacaaagggtattgctctactggcatttattttaggcgtgttttttggcaatatatcctcaccatcttgttctgaagaccatgaggaggtcatttctaaccaaacatcagttatagattctcaaaaaacagaaatagaaactttaaatagcaaattgtctgatgctgaaccatggttcaaaatgaaagacgacgaaaagaaagctattgaagctgaaaatcaacgtaaagctgaagaagctaaaaaagctgaagaagctaaaaaggctgaagaacaacgcaaaaaagaagaagaggagaagaaaggatatgatactggtattacttatgaccaattagctagaacacctgatgattataagtacaaaaaggtaaaatttgaaggtaaggttattcaa

SEQ ID NO: 3 - DNA, 680 bp, Clostridium difficile
  source 1..680: mol_type = genomic DNA
atgttttctaaaaaaaatgagggtaacgaatttagtaatgaaggaaaaggaagctctaagaaaataattaaattctttaagagcacaaagggtattgctctactggcatttattttggcgtgttttttggcaatatatcctcaccagcttgttctgaagaccatgaggaggtcatttctaatcaaacatcagttatagattctcaaaaaacagaaatagaaactttaaatagcaaattgtctgatgctgaaccatggttcaaaatgaaagacgacgaaaagaaagctattgaagctgaaaatcaacgtaaagctgaagaagctaaaaaggctgaagaacaacgtaaaaaagaagaagaagagaagaaaggatatgatactggtattacttatgaccaattagctagaacacctgatgattataagtacaaaaaggtaaaatttgaaggtaaggttattcaagttattgaagatggtgatgaggtgcaaataagattagctgtgtctggaaattatgataaggtcgtactatgtagttataaaaaatcaataactccttcaagagtgttagaggatgattacataactataagaggtataagtgctggaactataacttatgaatcaactatgggtggaaatataactataccagggatagctgtagagaaaataaattaa

SEQ ID NO: 4 - DNA, 681 bp, Clostridium difficile
  source 1..681: mol_type = genomic DNA
atgttttctaaaaaaaatgagggtaacgaatttagtaatgaaggaaaaggaagctctaagaaaataattaaattctttaagagcacaaagggtattgctctactggcatttatttttggcgtgttttttggcaatatatcctcaccagcttgttctgaagaccatgaggaggtcatttctaatcaaacatcagttatagattctcaaaaaacagaaatagaaactttaaatagcaaattgtctgatgctgaaccatggttcaaaatgaaagacgacgaaaagaaagctattgaagctgaaaatcaacgtaaagctgaagaagctaaaaaggctgaagaacaacgtaaaaaagaagaagaagagaagaaaggatatgatactggtattacttatgaccaattagctagaacacctgatgattataagtacaaaaaggtaaaatttgaaggtaaggttattcaagttattgaagatggtgatgaggtgcaaataagattagctgtgtctggaaattatgataaggtcgtactatgtagttataaaaaatcaataactccttcaagagtgttagaggatgattacataactataagaggtataagtgctggaactataacttatgaatcaactatgggtggaaatataactataccagggatagctgtagagaaaataaattaa

SEQ ID NO: 5 - DNA, 681 bp, Clostridium difficile
  source 1..681: mol_type = genomic DNA
atgttttctaaaaaaaatgagggtaacgaatttagtaatgaaggaaaaggaagctctaagaaaataattaaattctttaagagcacaaagggtattgctctactggcatttattttgggcgtgttttttggcaatatatcctcaccagcttgttctgaagaccatgaggaggtcatttctaattaaacatcagttatagattctcaaaaaacagaaatagaaactttaaatagcaaattgtctgatgctgaaccatggttcaaaatgaaagacgacgaaaagaaagctattgaagctgaaaatcaacgtaaagctgaagaagctaaaaaggctgaagaacaacgtaaaaaagaagaagaagagaagaaaggatatgatactggtattacttatgaccaattagctagaacacctgatgattataagtacaaaaaggtaaaatttgaaggtaaggttattcaagttattgaagatggtgatgaggtgcaaataagattagctgtgtctggaaattatgataaggtcgtactatgtagttataaaaaatcaataactccttcaagagtgttagaggatgattacataactataagaggtataagtgctggaactataacttatgaatcaactatgggtggaaatataactataccagggatagctgtagagaaaataaattaa

SEQ ID NO: 6 - DNA, 2631 bp, Clostridium difficile
  source 1..2631: mol_type = genomic DNA
atgaaagtacaaatgaggaataaaaaggtattaagttttttaacacttacagccatagttagtcaagcattagcatatcctgtatatgctcaaactagtacaagtagccattctgataataaaaaagaaattataaatgaagacatactcacaaacaacggattaatgggatattatttcacagacgaacactttaaagatttaaaattaatggcacccataaaagatggtaatttaaaatttgaagaaaagaaagtagataaacttctaaataaagacaaatcaaatgtaaaatctatacgatggacaggaagaataattccttctaaggatggtgaatatacattatcaactgatagagatgatattttaatgcaagtaaataatgagagtactatatcaaatacacttaaagttaatatgaaaaagggtaaagaatataaatttagaatagagctacaagataaaaatttaggttcaatagataatttatcatcaccaaatctttattgggaattagatggtattaagaaaattataccagcagaaaatttattcttaagagattattctaatatagaaaaaaatgatccatttatcccaaataacaatttctttgacccaaggttgatgtctgattgggaagacgaagatttggatacagataatgataatataccagattcatatgaacgaaatggatatactattaaggacttaattgcagttaagtgggaagatagctttgcagaacaaggctataagaaatatgtatcaaattatttagagtcaaatactgctggagatccatatacagattatgaaaaagcttcaggttcttttgacaaggctataaagaccgaagcaagagatccgttagttgcagcgtatccaattgttggagtaggtatggaaaaattaattatatctacaaatgaacatgcctctactgatcaaggtaaaactgtttccagagctactactaacagtaaaactgaatctaatacagctggtgtttctgttaatgtaggatatcaaaatggattcacagctaatgtaactacaaattattcccatacaacagataattcaactgccgttcaagatagtaatggagaatcatggaatactggattaagtataaacaaaggagaatctgcatatataaatgcaaatgttagatattacaacacaggtactgcacctatgtacaaagtgacaccaacaacaaatttagtgttagatggagatacattatcaactatcaaagcacaagaaaatcaaattggcaataatctatctcctggagatacttatcccaaaaaagggctttcacctctggctcttaacacaatggatcaatttagctctagactgattcctataaattatgatcaattaaaaaaattagatgctggaaagcaaattaaattagaaacaacacaagtaagtggaaattttggtacaaaaaatagttctggacaaatagtaacagaaggaaatagttggtcagactatataagtcaaattgacagtatttctgcatctattatattagatacagagaatgaatcttacgaaagaagagttactgctaaaaatttacagaatccagaagataaaacacctgaacttacaattggagaagcaattgaaaaagcttttggcgctactaaaaaagatggtttgttatattttaatgatataccaatagatgaaagttgtgttgaactcatatttgatgataatacagccaataagattaaagatagtttaaaaactttgtctgataaaaagatatataatgttaaacttgaaagaggaatgaatatacttataaaaacaccaacttactttactaattttgatgattacaataattaccctagtacatggagtaatgtcaatactacgaatcaagatggtttacaaggctcagcaaataaattaaatggtgagacaaagattaaaatacctatgtctaagctaaaaccttataaacgttatgtttttagtggatattcaaaggatcctttaacatctaattcaataattgtaaagataaaagcaaaagaagaaaaaacggattatttggtaccagaacaaggatatacaaagtttagttatgaatttgaaactactgaaaaagattcttctaatatagagataacattaattggtagtggtacaacatacttagataacttatctattacagaactgaatagtactcctgaaatacttaatgaaccagaagttaaaattccaactgaccaagaaataatagatgcacataaaatatattctgcagatttaaattttaatccaagtacaggaaatgcttatataaatggtatgtattttacaccaacacaaactaataaagaagctctcgattatatccaaaaatatagagttgaagctactttacaatattctggatttaaagatattggaactaaagataaagaaatgcgtaattatttaggagatccaaatcaacctaaaactaattatgtcaatcttaggagttattttacaggtggagaaaatattatgacatacaagaaattaagaatatatgcaattactccagatgatagagagttattagttcttagtgttgattag

SEQ ID NO: 7 - DNA, 8133 bp, Clostridium difficile
  source 1..8133: mol_type = genomic DNA
atgtctttaatatctaaagaagagttaataaaactcgcatatagcattagaccaagagaaaatgagtataaaactatactaactaatttagacgaatataataagttaactacaaacaataatgaaaataaatatttacaattaaaaaaactaaatgaatcaattgatgtttttatgaataaatataaaacttcaagcagaaatagagcactctctaatctaaaaaaagatatattaaaagaagtaattcttattaaaaattccaatacaagccctgtagaaaaaaatttacattttgtatggataggtggagaagtcagtgatattgctcttgaatacataaaacaatgggctgatattaatgcagaatataatattaaactgtggtatgatagtgaagcattcttagtaaatacactaaaaaaggctatagttgaatcttctaccactgaagcattacagctactagaggaagagattcaaaatcctcaatttgataatatgaaattttacaaaaaaaggatggaatttatatatgatagacaaaaaaggtttataaattattataaatctcaaatcaataaacctacagtacctacaatagatgatattataaagtctcatctagtatctgaatataatagagatgaaactgtattagaatcatatagaacaaattctttgagaaaaataaatagtaatcatgggatagatatcagggctaatagtttgtttacagaacaagagttattaaatatttatagtcaggagttgttaaatcgtggaaatttagctgcagcatctgacatagtaagattattagccctaaaaaattttggcggagtatatttagatgttgatatgcttccaggtattcactctgatttatttaaaacaatatctagacctagctctattggactagaccgttgggaaatgataaaattagaggctattatgaagtataaaaaatatataaataattatacatcagaaaactttgataaacttgatcaacaattaaaagataattttaaactcattatagaaagtaaaagtgaaaaatctgagatattttctaaattagaaaatttaaatgtatctgatcttgaaattaaaatagctttcgctttaggcagtgttataaatcaagccttgatatcaaaacaaggttcatatcttactaacctagtaatagaacaagtaaaaaatagatatcaatttttaaaccaacaccttaacccagccatagagtctgataataacttcacagatactactaaaatttttcatgattcattatttaattcagctaccgcagaaaactctatgtttttaacaaaaatagcaccatacttacaagtaggttttatgccagaagctcgctccacaataagtttaagtggtccaggagcttatgcgtcagcttactatgatttcataaatttacaagaaaatactatagaaaaaactttaaaagcatcagatttaatagaatttaaattcccagaaaataatctatctcaattgacagaacaagaaataaatagtctatggagctttgatcaagcaagtgcaaaatatcaatttgagaaatatgtaagagattatactggtggatctctttctgaagacaatggggtagactttaataaaaatactgccctcgacaaaaactatttattaaataataaaattccatcaaacaatgtagaagaagctggaagtaaaaattatgttcattatatcatacagttacaaggagatgatataagttatgaagcaacatgcaatttattttctaaaaatcctaaaaatagtattattatacaacgaaatatgaatgaaagtgcaaaaagctactttttaagtgatgatggagaatctattttagaattaaataaatataggatacctgaaagattaaaaaataaggaaaaagtaaaagtaacctttattggacatggtaaagatgaattcaacacaagcgaatttgctagattaagtgtagattcactttccaatgagataagttcatttttagataccataaaattagatatatcacctaaaaatgtagaagtaaacttacttggatgtaatatgtttagttatgattttaatgttgaagaaacttatcctgggaagttgctattaagtattatggacaaaattacttccactttacctgatgtaaataaaaattctattactataggagcaaatcaatatgaagtaagaattaatagtgagggaagaaaagaacttctggctcactcaggtaaatggataaataaagaagaagctattatgagcgatttatctagtaaagaatacattttttttgattctatagataataagctaaaagcaaagtccaagaatattccaggattagcatcaatatcagaagatataaaaacattattacttgatgcaagtgttagtcctgatacaaaatttattttaaataatcttaagcttaatattgaatcttctattggtgattacatttattatgaaaaattagagcctgttaaaaatataattcacaattctatagatgatttaatagatgagttcaatctacttgaaaatgtatctgatgaattatatgaattaaaaaaattaaataatctagatgagaagtatttaatatcttttgaagatatctcaaaaaataattcaacttactctgtaagatttattaacaaaagtaatggtgagtcagtttatgtagaaacagaaaaagaaattttttcaaaatatagcgaacatattacaaaagaaataagtactataaagaatagtataattacagatgttaatggtaatttattggataatatacagttagatcatacttctcaagttaatacattaaacgcagcattctttattcaatcattaatagattatagtagcaataaagatgtactgaatgatttaagtacctcagttaaggttcaactttatgctcaactatttagtacaggtttaaatactatatatgactctatccaattagtaaatttaatatcaaatgcagtaaatgatactataaatgtactacctacaataacagaggggatacctattgtatctactatattagacggaataaacttaggtgcagcaattaaggaattactagacgaacatgacccattactaaaaaaagaattagaagctaaggtgggtgttttagcaataaatatgtcattatctatagctgcaactgtagcttcaattgttggaataggtgctgaagttactattttcttattacctatagctggtatatctgcaggaataccttcattagttaataatgaattaatattgcatgataaggcaacttcagtggtaaactattttaatcatttgtctgaatctaaaaaatatggccctcttaaaacagaagatgataaaattttagttcctattgatgatttagtaatatcagaaatagattttaataataattcgataaaactaggaacatgtaatatattagcaatggaggggggatcaggacacacagtgactggtaatatagatcactttttctcatctccatctataagttctcatattccttcattatcaatttattctgcaataggtatagaaacagaaaatctagatttttcaaaaaaaataatgatgttacctaatgctccttcaagagtgttttggtgggaaactggagcagttccaggtttaagatcattggaaaatgacggaactagattacttgattcaataagagatttatacccaggtaaattttactggagattctatgcttttttcgattatgcaataactacattaaaaccagtttatgaagacactaatattaaaattaaactagataaagatactagaaacttcataatgccaactataactactaacgaaattagaaacaaattatcttattcatttgatggagcaggaggaacttactctttattattatcttcatatccaatatcaacgaatataaatttatctaaagatgatttatggatatttaatattgataatgaagtaagagaaatatctatagaaaatggtactattaaaaaaggaaagttaataaaagatgttttaagtaaaattgatataaataaaaataaacttattataggcaatcaaacaatagatttttcaggcgatatagataataaagatagatatatattcttgacttgtgagttagatgataaaattagtttaataatagaaataaatcttgttgcaaaatcttatagtttgttattgtctggggataaaaattatttgatatccaatttatctaatactattgagaaaatcaatactttaggcctagatagtaaaaatatagcgtacaattacactgatgaatctaataataaatattttggagctatatctaaaacaagtcaaaaaagcataatacattataaaaaagacagtaaaaatatattagaattttataatgacagtacattagaatttaacagtaaagattttattgctgaagatataaatgtatttatgaaagatgatattaatactataacaggaaaatactatgttgataataatactgataaaagtatagatttctctatttctttagttagtaaaaatcaagtaaaagtaaatggattatatttaaatgaatccgtatactcatcttaccttgattttgtgaaaaattcagatggacaccataatacttctaattttatgaatttatttttggacaatataagtttctggaaattgtttgggtttgaaaatataaattttgtaatcgataaatactttacccttgttggtaaaactaatcttggatatgtagaatttatttgtgacaataataaaaatatagatatatattttggtgaatggaaaacatcgtcatctaaaagcactatatttagcggaaatggtagaaatgttgtagtagagcctatatataatcctgatacgggtgaagatatatctacttcactagatttttcctatgaacctctctatggaatagatagatatataaataaagtattgatagcacctgatttatatacaagtttaataaatattaataccaattattattcaaatgagtactaccctgagattatagttcttaacccaaatacattccacaaaaaagtaaatataaatttagatagttcttcttttgagtataaatggtctacagaaggaagtgactttattttagttagatacttagaagaaagtaataaaaaaatattacaaaaaataagaatcaaaggtatcttatctaatactcaatcatttaataaaatgagtatagattttaaagatattaaaaaactatcattaggatatataatgagtaattttaaatcatttaattctgaaaatgaattagatagagatcatttaggatttaaaataatagataataaaacttattactatgatgaagatagtaaattagttaaaggattaatcaatataaataattcattattctattttgatcctatagaatttaacttagtaactggatggcaaactatcaatggtaaaaaatattattttgatataaatactggagcagctttaactagttataaaattattaatggtaaacacttttattttaataatgatggtgtgatgcagttgggagtatttaaaggacctgatggatttgaatattttgcacctgccaatactcaaaataataacatagaaggtcaggctatagtttatcaaagtaaattcttaactttgaatggcaaaaaatattattttgataatgactcaaaagcagtcactggatggagaattattaacaatgagaaatattactttaatcctaataatgctattgctgcagtcggattgcaagtaattgacaataataagtattatttcaatcctgacactgctatcatctcaaaaggttggcagactgttaatggtagtagatactactttgatactgataccgctattgcctttaatggttataaaactattgatggtaaacacttttattttgatagtgattgtgtagtgaaaataggtgtgtttagtacctctaatggatttgaatattttgcacctgctaatacttataataataacatagaaggtcaggctatagtttatcaaagtaaattcttaactttgaatggtaaaaaatattactttgataataactcaaaagcagttaccggatggcaaactattgatagtaaaaaatattactttaatactaacactgctgaagcagctactggatggcaaactattgatggtaaaaaatattactttaatactaacactgctgaagcagctactggatggcaaactattgatggtaaaaaatattactttaatactaacactgctatagcttcaactggttatacaattattaatggtaaacatttttattttaatactgatggtattatgcagataggagtgtttaaaggacctaatggatttgaatattttgcacctgctaatacggatgctaacaacatagaaggtcaagctatactttaccaaaatgaattcttaactttgaatagtaaaaaatattactttggtagtgactcaaaagcagttactggatggagaattattaacaataagaaatattactttaatcctaataatgctattgctgcaattcatctatgcactataaataatgacaagtattactttagttatgatggaattcttcaaaatggatatattactattgaaagaaataatttctattttgatgctaataatgaatctaaaatggtaacaggagtatttaaaggacctaatggatttgagtattttgcacctgctaatactcacaataataacatagaaggtcaggctatagtttaccagaacaaattcttaactttgaatggcaaaaaatattattttgataatgactcaaaagcagttactggatggcaaaccattgatggtaaaaaatattactttaatcttaacactgctgaagcagctactggatggcaaactattgatggtaaaaaatattactttaatcttaacactgctgaagcagctactggatggcaaactattgatggtaaaaaatattactttaatactaacactttcatagcctcaactggttatacaagtattaatggtaaacatttttattttaatactgatggtattatgcagataggagtgtttaaaggacctaatggatttgaatactttgcacctgctaatactcataataataacatagaaggtcaagctatactttaccaaaataaattcttaactttgaatggtaaaaaatattactttggtagtgactcaaaagcagttaccggattgcgaactattgatggtaaaaaatattactttaatactaacactgctgttgcagttactggatggcaaactattaatggtaaaaaatactactttaatactaacacttctatagcttcaactggttatacaattattagtggtaaacatttttattttaatactgatggtattatgcagataggagtgtttaaaggacctgatggatttgaatactttgcacctgctaatacagatgctaacaatatagaaggtcaagctatacgttatcaaaatagattcctatatttacatgacaatatatattattttggtaataattcaaaagcggctactggttgggtaactattgatggtaatagatattacttcgagcctaatacagctatgggtgcgaatggttataaaactattgataataaaaacttttactttagaaatggtttacctcagataggagtgtttaaagggtctaatggatttgaatactttgcacctgctaatacggatgctaacaatatagaaggtcaagctatacgttatcaaaatagattcctacatttacttggaaaaatatattactttggtaataattcaaaagcagttactggatggcaaactattaatggtaaagtatattactttatgcctgatactgctatggctgcagctggtggacttttcgagattgatggtgttatatatttctttggtgttgatggagtaaaagcccctgggatatatggctaa

SEQ ID NO: 8 - DNA, 7101 bp, Clostridium difficile
  source 1..7101: mol_type = genomic DNA
atgagtttagttaatagaaaacagttagaaaaaatggcaaatgtaagatttcgtactcaagaagatgaatatgttgcaatattggatgctttagaagaatatcataatatgtcagagaatactgtagtcgaaaaatatttaaaattaaaagatataaatagtttaacagatatttatatagatacatataaaaaatctggtagaaataaagccttaaaaaaatttaaggaatatctagttacagaagtattagagctaaagaataataatttaactccagttgagaaaaatttacattttgtttggattggaggtcaaataaatgacactgctattaattatataaatcaatggaaagatgtaaatagtgattataatgttaatgttttttatgatagtaatgcatttttgataaacacattgaaaaaaactgtagtagaatcagcaataaatgatacacttgaatcatttagagaaaacttaaatgaccctagatttgactataataaattcttcagaaaacgtatggaaataatttatgataaacagaaaaatttcataaactactataaagctcaaagagaagaaaatcctgaacttataattgatgatattgtaaagacatatctttcaaatgagtattcaaaggagatagatgaacttaatacctatattgaagaatccttaaataaaattacacagaatagtggaaatgatgttagaaactttgaagaatttaaaaatggagagtcattcaacttatatgaacaagagttggtagaaaggtggaatttagctgctgcttctgacatattaagaatatctgcattaaaagaaattggtggtatgtatttagatgttgatatgttaccaggaatacaaccagacttatttgagtctatagagaaacctagttcagtaacagtggatttttgggaaatgacaaagttagaagctataatgaaatacaaagaatatataccagaatatacctcagaacattttgacatgttagacgaagaagttcaaagtagttttgaatctgttctagcttctaagtcagataaatcagaaatattctcatcacttggtgatatggaggcatcaccactagaagttaaaattgcatttaatagtaagggtattataaatcaagggctaatttctgtgaaagactcatattgtagcaatttaatagtaaaacaaatcgagaatagatataaaatattgaataatagtttaaatccagctattagcgaggataatgattttaatactacaacgaatacctttattgatagtataatggctgaagctaatgcagataatggtagatttatgatggaactaggaaagtatttaagagttggtttcttcccagatgttaaaactactattaacttaagtggccctgaagcatatgcggcagcttatcaagatttattaatgtttaaagaaggcagtatgaatatccatttgatagaagctgatttaagaaactttgaaatctctaaaactaatatttctcaatcaactgaacaagaaatggctagcttatggtcatttgacgatgcaagagctaaagctcaatttgaagaatataaaaggaattattttgaaggttctcttggtgaagatgataatcttgatttttctcaaaatatagtagttgacaaggagtatcttttagaaaaaatatcttcattagcaagaagttcagagagaggatatatacactatattgttcagttacaaggagataaaattagttatgaagcagcatgtaacttatttgcaaagactccttatgatagtgtactgtttcagaaaaatatagaagattcagaaattgcatattattataatcctggagatggtgaaatacaagaaatagacaagtataaaattccaagtataatttctgatagacctaagattaaattaacatttattggtcatggtaaagatgaatttaatactgatatatttgcaggttttgatgtagattcattatccacagaaatagaagcagcaatagatttagctaaagaggatatttctcctaagtcaatagaaataaatttattaggatgtaatatgtttagctactctatcaacgtagaggagacttatcctggaaaattattacttaaagttaaagataaaatatcagaattaatgccatctataagtcaagactctattatagtaagtgcaaatcaatatgaagttagaataaatagtgaaggaagaagagaattattggatcattctggtgaatggataaataaagaagaaagtattataaaggatatttcatcaaaagaatatatatcatttaatcctaaagaaaataaaattacagtaaaatctaaaaatttacctgagctatctacattattacaagaaattagaaataattctaattcaagtgatattgaactagaagaaaaagtaatgttaacagaatgtgagataaatgttatttcaaatatagatacgcaaattgttgaggaaaggattgaagaagctaagaatttaacttctgactctattaattatataaaagatgaatttaaactaatagaatctatttctgatgcactatgtgacttaaaacaacagaatgaattagaagattctcattttatatcttttgaggacatatcagagactgatgagggatttagtataagatttattaataaagaaactggagaatctatatttgtagaaactgaaaaaacaatattctctgaatatgctaatcatataactgaagagatttctaagataaaaggtactatatttgatactgtaaatggtaagttagtaaaaaaagtaaatttagatactacacacgaagtaaatactttaaatgctgcattttttatacaatcattaatagaatataatagttctaaagaatctcttagtaatttaagtgtagcaatgaaagtccaagtttacgctcaattatttagtactggtttaaatactattacagatgcagccaaagttgttgaattagtatcaactgcattagatgaaactatagacttacttcctacattatctgaaggattacctataattgcaactattatagatggtgtaagtttaggtgcagcaatcaaagagctaagtgaaacgagtgacccattattaagacaagaaatagaagctaagataggtataatggcagtaaatttaacaacagctacaactgcaatcattacttcatctttggggatagctagtggatttagtatacttttagttcctttagcaggaatttcagcaggtataccaagcttagtaaacaatgaacttgtacttcgagataaggcaacaaaggttgtagattattttaaacatgtttcattagttgaaactgaaggagtatttactttattagatgataaaataatgatgccacaagatgatttagtgatatcagaaatagattttaataataattcaatagttttaggtaaatgtgaaatctggagaatggaaggtggttcaggtcatactgtaactgatgatatagatcacttcttttcagcaccatcaataacatatagagagccacacttatctatatatgacgtattggaagtacaaaaagaagaacttgatttgtcaaaagatttaatggtattacctaatgctccaaatagagtatttgcttgggaaacaggatggacaccaggtttaagaagcttagaaaatgatggcacaaaactgttagaccgtataagagataactatgaaggtgagttttattggagatattttgcttttatagctgatgctttaataacaacattaaaaccaagatatgaagatactaatataagaataaatttagatagtaatactagaagttttatagttccaataataactacagaatatataagagaaaaattatcatattctttctatggttcaggaggaacttatgcattgtctctttctcaatataatatgggtataaatatagaattaagtgaaagtgatgtttggattatagatgttgataatgttgtgagagatgtaactatagaatctgataaaattaaaaaaggtgatttaatagaaggtattttatctacactaagtattgaagagaataaaattatcttaaatagccatgagattaatttttctggtgaggtaaatggaagtaatggatttgtttctttaacattttcaattttagaaggaataaatgcaattatagaagttgatttattatctaaatcatataaattacttatttctggcgaattaaaaatattgatgttaaattcaaatcatattcaacagaaaatagattatataggattcaatagcgaattacagaaaaatataccatatagctttgtagatagtgaaggaaaagagaatggttttattaatggttcaacaaaagaaggtttatttgtatctgaattacctgatgtagttcttataagtaaggtttatatggatgatagtaagccttcatttggatattatagtaataatttgaaagatgtcaaagttataactaaagataatgttaatatattaacaggttattatcttaaggatgatataaaaatctctctttctttgactctacaagatgaaaaaactataaagttaaatagtgtgcatttagatgaaagtggagtagctgagattttgaagttcatgaatagaaaaggtaatacaaatacttcagattctttaatgagctttttagaaagtatgaatataaaaagtattttcgttaatttcttacaatctaatattaagtttatattagatgctaattttataataagtggtactacttctattggccaatttgagtttatttgtgatgaaaatgataatatacaaccatatttcattaagtttaatacactagaaactaattatactttatatgtaggaaatagacaaaatatgatagtggaaccaaattatgatttagatgattctggagatatatcttcaactgttatcaatttctctcaaaagtatctttatggaatagacagttgtgttaataaagttgtaatttcaccaaatatttatacagatgaaataaatataacgcctgtatatgaaacaaataatacttatccagaagttattgtattagatgcaaattatataaatgaaaaaataaatgttaatatcaatgatctatctatacgatatgtatggagtaatgatggtaatgattttattcttatgtcaactagtgaagaaaataaggtgtcacaagttaaaataagattcgttaatgtttttaaagataagactttggcaaataagctatcttttaactttagtgataaacaagatgtacctgtaagtgaaataatcttatcatttacaccttcatattatgaggatggattgattggctatgatttgggtctagtttctttatataatgagaaattttatattaataactttggaatgatggtatctggattaatatatattaatgattcattatattattttaaaccaccagtaaataatttgataactggatttgtgactgtaggcgatgataaatactactttaatccaattaatggtggagctgcttcaattggagagacaataattgatgacaaaaattattatttcaaccaaagtggagtgttacaaacaggtgtatttagtacagaagatggatttaaatattttgccccagctaatacacttgatgaaaacctagaaggagaagcaattgattttactggaaaattaattattgacgaaaatatttattattttgatgataattatagaggagctgtagaatggaaagaattagatggtgaaatgcactattttagcccagaaacaggtaaagcttttaaaggtctaaatcaaataggtgatgataaatactatttcaattctgatggagttatgcaaaaaggatttgttagtataaatgataataaacactattttgatgattctggtgttatgaaagtaggttacactgaaatagatggcaagcatttctactttgctgaaaacggagaaatgcaaataggagtatttaatacagaagatggatttaaatattttgctcatcataatgaagatttaggaaatgaagaaggtgaagaaatctcatattctggtatattaaatttcaataataaaatttactattttgatgattcatttacagctgtagttggatggaaagatttagaggatggttcaaagtattattttgatgaagatacagcagaagcatatataggtttgtcattaataaatgatggtcaatattattttaatgatgatggaattatgcaagttggatttgtcactataaatgataaagtcttctacttttctgactctggaattatagaatctggagtacaaaacatagatgacaattatttctatatagatgataatggtatagttcaaattggtgtatttgatacttcagatggatataaatattttgcacctgctaatactgtaaatgataatatttacggacaagcagttgaatatagtggtttagttagagttggggaagatgtatattattttggagaaacatatacaattgagactggatggatatatgatatggaaaatgaaagtgataaatattatttcaatccagaaactaaaaaagcatgcaaaggtattaatttaattgatgatataaaatattattttgatgagaagggcataatgagaacgggtcttatatcatttgaaaataataattattactttaatgagaatggtgaaatgcaatttggttatataaatatagaagataagatgttctattttggtgaagatggtgtcatgcagattggagtatttaatacaccagatggatttaaatactttgcacatcaaaatactttggatgagaattttgagggagaatcaataaactatactggttggttagatttagatgaaaagagatattattttacagatgaatatattgcagcaactggttcagttattattgatggtgaggagtattattttgatcctgatacagctcaattagtgattagtgaatag

SEQ ID NO: 9 - DNA, 1392 bp, Clostridium difficile
  source 1..1392: mol_type = genomic DNA
atgaaaaaatttagaaaacataaaagtattagtaattgtatatctatattgttgatattatatctaactttaggtagtttgttacctaataacatttatgcacaagacttacaaagctatagtgaaaaagtttgcaatactacttacaaggctcctatagaaagaccagaagattttcttaaagataaagaaagggctaaagaatgggaaagaaaagaagcagaaagaatagagcaaaaacttgaaagatctgaaaaagaagcattagaatcatataaaaaagattctgtagaaataaataaatattctcagacaagaaattatttttatgattatcaaatagaagcaaattctcgagaaaaagaatatagagaacttcgaaatgctatatcaaaaaataaaatagataaacctatgtatgtctattattttgaatctccagaaaaatttgcatttaataaagtaataagaacagaaaatcaaaacgaaatttcattagaaaaatttaatgagtttaaagaaactatacaaaacaaattatttaagcaagatggatttaaagaaatttctttatatgaacctggaaaaggtgatgaagaacctacaccattacttatgcacttaaaattacctagaaatactggtatgttaccatatacaaatactaacaatgtaagtacattaatagagcaaggatatagtataaaaatagataaaattgttcgtatagttatagatgggaaacattatattaaagcagaagcatctgttgtaagtagtcttgattttaaggatgatgtaagtaagggggactcttggggtaaagcaaattataatgattggagtaataaattaacacctaatgaacttgctgatgtaaatgattatatgcgtggaggatatactgcaattaataattatttaatatcaaatggtccagtaaataaccctaacccagaattagattctaaaatcacaaacattgaaaatgcattaaaacgtgaacctattccaactaatttaactgtatatagaagatctggtcctcaagaatttggtttaacccttacttcccctgaatatgattttaacaaaccagaaaatatagatgcttttaaatcaaaatgggaaggacaaacactgtcttatccaaactttattagtactagtattggtagtgtgaatatgagtgcatttgctaaaagaaaaatagtactacgtataactatacctaaaggttctcctggagcctatctatcagctattccaggttatgcaggtgaatatgaagtacttttaaatcatggaagcaaatttaaaatcagtaaaattgattcttacaaagatggcgctataacaaaattaattgttgatgcaacattgataccttaa

SEQ ID NO: 10 - DNA, 35 bp, synthetic construct
  misc_feature 1..35: note = Exemplary cdtB FRET cassette
  modified_base 1: mod_base = OTHER; note = Redmond Red fluorophore labeled
  modified_base 4: mod_base = OTHER; note = Eclipse Quencher
  modified_base 35: mod_base = OTHER; note = 3'-terminal Hexanediol
  source 1..35: mol_type = other DNA
tctagccggttttccggctgagacgtccgtggcct

SEQ ID NO: 11 - DNA, 34 bp, synthetic construct
  misc_feature 1..34: note = Exemplary tcdC FRET cassette
  modified_base 1: mod_base = OTHER; note = Hexochloro-Fluorescein labeled
  modified_base 4: mod_base = OTHER; note = Blackberry Quencher 650 dT
  modified_base 34: mod_base = OTHER; note = 3'-terminal Hexanediol
  source 1..34: mol_type = other DNA
tcttagccggttttccggctgagacctcggcgcg

SEQ ID NO: 12 - DNA, 36 bp, synthetic construct
  misc_feature 1..36: note = Exemplary tcdB/tcdA FRET cassette
  modified_base 1: mod_base = OTHER; note = Fluorescein labeled
  modified_base 4: mod_base = OTHER; note = Blackberry Quencher 650 dT
  modified_base 36: mod_base = OTHER; note = 3'-terminal Hexanediol
  source 1..36: mol_type = other DNA
tcttagccggttttccggctgagactccgcgtccgt

SEQ ID NO: 13 - DNA, 53 bp, synthetic construct
  misc_feature 1..53: note = cdtA Forward Primer
  source 1..53: mol_type = other DNA
gagcaaggatatagtataaaaatagataaaattgttcgtatagttatagatgg

SEQ ID NO: 14 - DNA, 38 bp, synthetic construct
  misc_feature 1..38: note = cdtA Invader
  source 1..38: mol_type = other DNA
tatattaaagcagaagcatctgttgtaagtagtcttgt

SEQ ID NO: 15 - DNA, 38 bp, synthetic construct
  misc_feature 1..38: note = cdtA Invader
  source 1..38: mol_type = other DNA
tatattaaagcagaagcatctgttgtaagtagtcttgc

SEQ ID NO: 16 - DNA, 27 bp, synthetic construct
  misc_feature 1..27: note = THS for cdtA probe
  source 1..27: mol_type = other DNA
attttaaggatgatgtaagtaagggag

SEQ ID NO: 17 - DNA, 27 bp, synthetic construct
  misc_feature 1..27: note = THS for cdtA probe
  source 1..27: mol_type = other DNA
cgattttaaggatgatgtaagtaaggg

SEQ ID NO: 18 - DNA, 37 bp, synthetic construct
  misc_feature 1..37: note = cdtA Primary Probe
  modified_base 37: mod_base = OTHER; note = 3'-terminal Hexanediol
  source 1..37: mol_type = other DNA
cgcgaggccgattttaaggatgatgtaagtaagggag

SEQ ID NO: 19 - DNA, 39 bp, synthetic construct
  misc_feature 1..39: note = cdtA Primary Probe
  modified_base 39: mod_base = OTHER; note = 3'-terminal Hexanediol
  source 1..39: mol_type = other DNA
aggccacggacgattttaaggatgatgtaagtaagggag

SEQ ID NO: 20 - DNA, 37 bp, synthetic construct
  misc_feature 1..37: note = cdtA Primary Probe
  modified_base 37: mod_base = OTHER; note = 3'-terminal Hexanediol
  source 1..37: mol_type = other DNA
aggccacggacgattttaaggatgatgtaagtaaggg

SEQ ID NO: 21 - DNA, 32 bp, synthetic construct
  misc_feature 1..32: note = cdtA Reverse Primer
  source 1..32: mol_type = other DNA
actccaatcactataatttgctttaccccaag

SEQ ID NO: 22 - DNA, 36 bp, synthetic construct
  misc_feature 1..36: note = cdtA Reverse Primer
  source 1..36: mol_type = other DNA
cgcatataatcatttacatcagcaagttcattaggt

SEQ ID NO: 23 - DNA, 25 bp, synthetic construct
  misc_feature 1..25: note = cdtA Reverse Primer
  source 1..25: mol_type = other DNA
atttgctttaccccaagagtctccc

SEQ ID NO: 24 - DNA, 21 bp, synthetic construct
  misc_feature 1..21: note = cdtA Reverse Primer
  source 1..21: mol_type = other DNA
gctttaccccaagagtctccc

SEQ ID NO: 25 - DNA, 36 bp, synthetic construct
  misc_feature 1..36: note = cdtA Reverse Primer
  source 1..36: mol_type = other DNA
actccaatcactataatttgctttaccccaagagtc

SEQ ID NO: 26 - DNA, 32 bp, synthetic construct
  misc_feature 1..32: note = cdtA Reverse Primer
  source 1..32: mol_type = other DNA
caatcactataatttgctttaccccaagagtc

SEQ ID NO: 27 - DNA, 42 bp, synthetic construct
  misc_feature 1..42: note = cdtA Reverse Primer
  source 1..42: mol_type = other DNA
aggtgttaatttattactccaatcactataatttgctttacc

SEQ ID NO: 28 - DNA, 39 bp, synthetic construct
  misc_feature 1..39: note = cdtA Reverse Primer
  source 1..39: mol_type = other DNA
tgttaatttattactccaatcactataatttgctttacc

SEQ ID NO: 29 - DNA, 43 bp, synthetic construct
  misc_feature 1..43: note = cdtA Reverse Primer
  source 1..43: mol_type = other DNA
gttcattaggtgttaatttattactccaatcactataatttgc

SEQ ID NO: 30 - DNA, 32 bp, synthetic construct
  misc_feature 1..32: note = Forward Primer
  source 1..32: mol_type = other DNA
gttgatgtctgattgggaagacgaagatttgg

SEQ ID NO: 31 - DNA, 27 bp, synthetic construct
  misc_feature 1..27: note = Forward Primer
  source 1..27: mol_type = other DNA
tttgacccaaggttgatgtctgattgg

SEQ ID NO: 32 - DNA, 24 bp, synthetic construct
  misc_feature 1..24: note = Forward Primer
  source 1..24: mol_type = other DNA
gacccaaggttgatgtctgattgg

SEQ ID NO: 33 - DNA, 28 bp, synthetic construct
  misc_feature 1..28: note = Forward Primer
  source 1..28: mol_type = other DNA
ctttgacccaaggttgatgtctgattgg

SEQ ID NO: 34 - DNA, 32 bp, synthetic construct
  misc_feature 1..32: note = THS for cdtB probe
  source 1..32: mol_type = other DNA
cgttcatatgaatctggtatattatcattatc

SEQ ID NO: 35 - DNA, 32 bp, synthetic construct
  misc_feature 1..32: note = THS for cdtB probe
  source 1..32: mol_type = other DNA
gttcatatgaatctggtatattatcattatct

SEQ ID NO: 36 - DNA, 26 bp, synthetic construct
  misc_feature 1..26: note = THS for cdtB probe
  source 1..26: mol_type = other DNA
cgttcatatgaatctggtatattatc

SEQ ID NO: 37 - DNA, 44 bp, synthetic construct
  misc_feature 1..44: note = cdtB Primary Probe
  misc_feature 1..12: note = RNA
  misc_feature 13..44: note = DNA
  modified_base 44: mod_base = OTHER; note = 3'-terminal Hexanediol
  source 1..44: mol_type = other DNA
aggccacggacgcgttcatatgaatctggtatattatcattatc

SEQ ID NO: 38 - DNA, 44 bp, synthetic construct
  misc_feature 1..44: note = cdtB Primary Probe
  misc_feature 1..12: note = RNA
  misc_feature 13..44: note = DNA
  modified_base 44: mod_base = OTHER; note = 3'-terminal Hexanediol
  source 1..44: mol_type = other DNA
aggccacggacggttcatatgaatctggtatattatcattatct

SEQ ID NO: 39 - DNA, 38 bp, synthetic construct
  misc_feature 1..38: note = cdtB Primary Probe
  misc_feature 1..12: note = RNA
  misc_feature 13..38: note = DNA
  modified_base 38: mod_base = OTHER; note = 3'-terminal Hexanediol
  source 1..38: mol_type = other DNA
aggccacggacgcgttcatatgaatctggtatattatc

SEQ ID NO: 40 - DNA, 40 bp, synthetic construct
  misc_feature 1..40: note = cdtB Reverse iPrimer
  source 1..40: mol_type = other DNA
cacttaactgcaattaagtccttaatagtatatccatttc

SEQ ID NO: 41 - DNA, 40 bp, synthetic construct
  misc_feature 1..40: note = cdtB Reverse iPrimer
  source 1..40: mol_type = other DNA
acttaactgcaattaagtccttaatagtatatccatttcg

SEQ ID NO: 42 - DNA, 40 bp, synthetic construct
  misc_feature 1..40: note = cdtB Invader
  source 1..40: mol_type = other DNA
acttaactgcaattaagtccttaatagtatatccatttct

SEQ ID NO: 43 - DNA, 21 bp, synthetic construct
  misc_feature 1..21: note = THS for cdtB probe
  source 1..21: mol_type = other DNA
ggaagacgaagatttggatac

SEQ ID NO: 44 - DNA, 33 bp, synthetic construct
  misc_feature 1..33: note = THS for cdtB probe
  source 1..33: mol_type = other DNA
gatacagataatgataatataccagattcatat

SEQ ID NO: 45 - DNA, 32 bp, synthetic construct; identical to SEQ ID NO: 35
  misc_feature 1..32: note = THS for cdtB probe
  source 1..32: mol_type = other DNA

SEQ ID NO: 46 - DNA, 31 bp, synthetic construct
  misc_feature 1..31: note = cdtB Primary Probe
  modified_base 31: mod_base = OTHER; note = 3'-terminal Hexanediol
  source 1..31: mol_type = other DNA
cgcgaggccgggaagacgaagatttggatac

SEQ ID NO: 47 - DNA, 43 bp, synthetic construct
  misc_feature 1..43: note = cdtB Primary Probe
  modified_base 43: mod_base = OTHER; note = 3'-terminal Hexanediol
  source 1..43: mol_type = other DNA
cgcgaggccggatacagataatgataatataccagattcatat

SEQ ID NO: 48 - DNA, 42 bp, synthetic construct
  misc_feature 1..42: note = cdtB Primary Probe
  modified_base 42: mod_base = OTHER; note = 3'-terminal Hexanediol
  source 1..42: mol_type = other DNA
cgcgaggccggttcatatgaatctggtatattatcattatct

SEQ ID NO: 49 - DNA, 37 bp, synthetic construct
  misc_feature 1..37: note = cdtB Reverse Primer
  source 1..37: mol_type = other DNA
cataatctgtatatggatctccagcagtatttgactc

SEQ ID NO: 50 - DNA, 44 bp, synthetic construct
  misc_feature 1..44: note = cdtB Reverse Primer
  source 1..44: mol_type = other DNA
gactctaaataatttgatacatatttcttatagccttgttctgc

SEQ ID NO: 51 - DNA, 21 bp, synthetic construct
  misc_feature 1..21: note = cdtB Primary Probe
  source 1..21: mol_type = other DNA
ggaagacgaagatttggatac

SEQ ID NO: 52 - DNA, 22 bp, synthetic construct
  misc_feature 1..22: note = cdtB Primary Probe
  source 1..22: mol_type = other DNA
ggaagacgaagatttggataca

SEQ ID NO: 53 - DNA, 33 bp, synthetic construct
  misc_feature 1..33: note = cdtB Primary Probe
  modified_base 33: mod_base = OTHER; note = 3'-terminal Hexanediol
  source 1..33: mol_type = other DNA
aggccacggacgggaagacgaagatttggatac

SEQ ID NO: 54 - DNA, 34 bp, synthetic construct
  misc_feature 1..34: note = cdtB Primary Probe
  modified_base 34: mod_base = OTHER; note = 3'-terminal Hexanediol
  source 1..34: mol_type = other DNA
aggccacggacgggaagacgaagatttggataca

SEQ ID NO: 55 - DNA, 21 bp, synthetic construct
  misc_feature 1..21: note = cdtB Forward Primer
  source 1..21: mol_type = other DNA
gatccatttatcccaaataac

SEQ ID NO: 56 - DNA, 24 bp, synthetic construct
  misc_feature 1..24: note = cdtB Reverse Primer
  source 1..24: mol_type = other DNA
catatttcttatagccttgttctg

SEQ ID NO: 57 - DNA, 21 bp, synthetic construct
  misc_feature 1..21: note = THS for tcdA Probe
  source 1..21: mol_type = other DNA
gaataaacttaggtgcagcaa

SEQ ID NO: 58 - DNA, 32 bp, synthetic construct
  misc_feature 1..32: note = tcdA Primary Probe
  modified_base 32: mod_base = OTHER; note = 3'-terminal Hexanediol
  source 1..32: mol_type = other DNA
tccgcgcgtccgaataaacttaggtgcagcaa

SEQ ID NO: 59 - DNA, 23 bp, synthetic construct
  misc_feature 1..23: note = tcdA Forward Primer
  source 1..23: mol_type = other DNA
ctttatgctcaactatttagtac

SEQ ID NO: 60 - DNA, 27 bp, synthetic construct
  misc_feature 1..27: note = tcdA Forward Primer
  source 1..27: mol_type = other DNA
cagtaaatgatactataaatgtactac

SEQ ID NO: 61 - DNA, 19 bp, synthetic construct
  misc_feature 1..19: note = THS for tcdA Probe
  source 1..19: mol_type = other DNA
ctagacgaacatgacccat

SEQ ID NO: 62 - DNA, 31 bp, synthetic construct
  misc_feature 1..31: note = tcdA Primary Probe
  misc_feature 1..12: note = RNA
  misc_feature 13..31: note = DNA
  modified_base 31: mod_base = OTHER; note = 3'-terminal Hexanediol
  source 1..31: mol_type = other DNA
acggacgcggagctagacgaacatgacccat

SEQ ID NO: 63 - DNA, 31 bp, synthetic construct
  misc_feature 1..31: note = tcdA Invader
  misc_feature 1..30: note = DNA
  misc_feature 31: note = RNA
  source 1..31: mol_type = other DNA
ataaacttaggtgcagcaattaaggaattaa

SEQ ID NO: 64 - DNA, 37 bp, synthetic construct
  misc_feature 1..37: note = tcdA Forward iPrimer
  source 1..37: mol_type = other DNA
cagaggggatacctattgtatctactatattagacgg

SEQ ID NO: 65 - DNA, 35 bp, synthetic construct
  misc_feature 1..35: note = tcdA Forward iPrimer
  source 1..35: mol_type = other DNA
gaggggatacctattgtatctactatattagacgg

SEQ ID NO: 66 - DNA, 19 bp, synthetic construct
  misc_feature 1..19: note = THS for tcdA Probe
  source 1..19: mol_type = other DNA
ctagacgaacatgacccat

SEQ ID NO: 67 - DNA, 21 bp, synthetic construct
  misc_feature 1..21: note = THS for tcdA Probe
  source 1..21: mol_type = other DNA
gaataaacttaggtgcagcaa

SEQ ID NO: 68 - DNA, 29 bp, synthetic construct
  misc_feature 1..29: note = tcdA Primary Probe
  misc_feature 1..10: note = RNA
  misc_feature 11..29: note = DNA
  modified_base 29: mod_base = OTHER; note = 3'-terminal Hexanediol
  source 1..29: mol_type = other DNA
cgcgccgaggctagacgaacatgacccat

SEQ ID NO: 69 - DNA, 33 bp, synthetic construct
  misc_feature 1..33: note = tcdA Primary Probe
  misc_feature 1..12: note = RNA
  misc_feature 13..33: note = DNA
  modified_base 33: mod_base = OTHER; note = 3'-terminal Hexanediol
  source 1..33: mol_type = other DNA
acggacgcggaggaataaacttaggtgcagcaa

SEQ ID NO: 70 - DNA, 35 bp, synthetic construct
  misc_feature 1..35: note = tcdA Reverse Primer
  source 1..35: mol_type = other DNA
gtaacttcagcacctattccaacaattgaagctac

SEQ ID NO: 71 - DNA, 30 bp, synthetic construct
  misc_feature 1..30: note = tcdA Reverse Primer
  source 1..30: mol_type = other DNA
agtaatgggtcatgttcgtctagtaattcc

SEQ ID NO: 72 - DNA, 35 bp, synthetic construct
  misc_feature 1..35: note = tcdA Reverse Primer
  source 1..35: mol_type = other DNA
gacatatttattgctaaaacacccaccttagcttc

SEQ ID NO: 73 - DNA, 24 bp, synthetic construct
  misc_feature 1..24: note = tcdA Reverse Primer
  source 1..24: mol_type = other DNA
gatataccagctataggtaataag

SEQ ID NO: 74 - DNA, 29 bp, synthetic construct
  misc_feature 1..29: note = tcdC 117 Invader
  misc_feature 1..28: note = DNA
  misc_feature 29: note = RNA
  source 1..29: mol_type = other DNA
ggtgaggatatattgccaaaaaacacgcg

SEQ ID NO: 75 - DNA, 33 bp, synthetic construct
  misc_feature 1..33: note = tcdC 117 Invader
  misc_feature 1..32: note = DNA
  misc_feature 33: note = RNA
  source 1..33: mol_type = other DNA
acaaagggtattgctctactggcatttattttc

SEQ ID NO: 76 - DNA, 33 bp, synthetic construct
  misc_feature 1..33: note = tcdC 117 Iprimer
  source 1..33: mol_type = other DNA
acaaagggtattgctctactggcatttattttn

SEQ ID NO: 77-85 - skipped (9 entries)

SEQ ID NO: 86 - DNA, 29 bp, synthetic construct
  misc_feature 1..29: note = tcdC 117del Invader
  misc_feature 1..28: note = DNA
  misc_feature 29: note = RNA
  source 1..29: mol_type = other DNA
ggtgaggatatattgccaaaaaacacacg

SEQ ID NO: 87 - DNA, 30 bp, synthetic construct
  misc_feature 1..30: note = tcdC 117del Reverse iPrimer
  source 1..30: mol_type = other DNA
ggtgaggatatattgccaaaaaacacacca

SEQ ID NO: 88 - DNA, 29 bp, synthetic construct
  misc_feature 1..29: note = tcdC 117del Reverse iPrimer
  source 1..29: mol_type = other DNA
ggtgaggatatattgccaaaaaacacacc

SEQ ID NO: 89 - DNA, 28 bp, synthetic construct
  misc_feature 1..28: note = tcdC 117del Reverse iPrimer
  source 1..28: mol_type = other DNA
ggtgaggatatattgccaaaaaacacgc

SEQ ID NO: 90 - DNA, 28 bp, synthetic construct
  misc_feature 1..28: note = tcdC 117del Reverse iPrimer
  source 1..28: mol_type = other DNA
ggtgaggatatattgccaaaaaacacac

SEQ ID NO: 91 - DNA, 27 bp, synthetic construct
  misc_feature 1..27: note = tcdC 117del Reverse iPrimer
  source 1..27: mol_type = other DNA
ggtgaggatatattgccaaaaaacacg

SEQ ID NO: 92 - DNA, 27 bp, synthetic construct
  misc_feature 1..27: note = tcdC 117del Reverse iPrimer
  source 1..27: mol_type = other DNA
ggtgaggatatattgccaaaaaacaca

SEQ ID NO: 93 - DNA, 21 bp, synthetic construct
  misc_feature 1..21: note = THS for tcdC 117del Probe
  source 1..21: mol_type = other DNA
caaaataaatgccagtagagc

SEQ ID NO: 94 - DNA, 25 bp, synthetic construct
  misc_feature 1..25: note = THS for tcdC 117del Probe
  source 1..25: mol_type = other DNA
aaaataaatgccagtagagcaatat

SEQ ID NO: 95 - DNA, 26 bp, synthetic construct
  misc_feature 1..26: note = THS for tcdC 117del Probe
  source 1..26: mol_type = other DNA
aaaataaatgccagtagagcaatatc

SEQ ID NO: 96 - DNA, 20 bp, synthetic construct
  misc_feature 1..20: note = THS for tcdC 117del Probe
  source 1..20: mol_type = other DNA
caaaataaatgccagtagag

SEQ ID NO: 97 - DNA, 19 bp, synthetic construct
  misc_feature 1..19: note = THS for tcdC 117del Probe
  source 1..19: mol_type = other DNA
caaaataaatgccagtaga

SEQ ID NO: 98 - DNA, 23 bp, synthetic construct
  misc_feature 1..23: note = THS for tcdC 117del Probe
  source 1..23: mol_type = other DNA
caaaataaatgccagtagagcaa

SEQ ID NO: 99 - DNA, 26 bp, synthetic construct
  misc_feature 1..26: note = THS for tcdC 117del Probe
  source 1..26: mol_type = other DNA
caaaataaatgccagtagagcaatac

SEQ ID NO: 100 - DNA, 22 bp, synthetic construct
  misc_feature 1..22: note = THS for tcdC 117del Probe
  source 1..22: mol_type = other DNA
caaataaatgccagtagagcaa

SEQ ID NO: 101 - DNA, 21 bp, synthetic construct
  misc_feature 1..21: note = THS for tcdC 117del Probe
  source 1..21: mol_type = other DNA
caataaatgccagtagagcaa

SEQ ID NO: 102 - DNA, 20 bp, synthetic construct
  misc_feature 1..20: note = THS for tcdC 117del Probe
  source 1..20: mol_type = other DNA
cataaatgccagtagagcaa

SEQ ID NO: 103 - DNA, 23 bp, synthetic construct
  misc_feature 1..23: note = THS for tcdC 117del Probe w/methoxy
  misc_feature 1..5: note = DNA
  misc_feature 6: note = RNA
  misc_feature 7..23: note = DNA
  source 1..23: mol_type = other DNA
caaaataaatgccagtagagcaa

SEQ ID NO: 104 - DNA, 23 bp, synthetic construct
  misc_feature 1..23: note = THS for tcdC 117del Probe w/methoxy
  modified_base 4: mod_base = OTHER; note = n is 2-O-Methyladenosine
  source 1..23: mol_type = other DNA
caanataaatgccagtagagcaa

SEQ ID NO: 105 - DNA, 23 bp, synthetic construct
  misc_feature 1..23: note = THS for tcdC 117del Probe w/methoxy
  modified_base 3: mod_base = OTHER; note = n is 2-O-Methyladenosine
  source 1..23: mol_type = other DNA
canaataaatgccagtagagcaa

SEQ ID NO: 106 - DNA, 22 bp, synthetic construct
  misc_feature 1..22: note = THS for tcdC 117del Probe w/methoxy
  misc_feature 1..4: note = DNA
  misc_feature 5: note = RNA
  misc_feature 6..22: note = DNA
  source 1..22: mol_type = other DNA
caaataaatgccagtagagcaa

SEQ ID NO: 107 - DNA, 21 bp, synthetic construct
  misc_feature 1..21: note = THS for tcdC 117del Probe w/methoxy
  misc_feature 1..3: note = DNA
  misc_feature 4: note = RNA
  misc_feature 5..21: note = DNA
  source 1..21: mol_type = other DNA
caataaatgccagtagagcaa

SEQ ID NO: 108 - DNA, 20 bp, synthetic construct
  misc_feature 1..2: note = THS for tcdC 117del Probe w/methoxy, DNA
  misc_feature 3: note = RNA
  misc_feature 4..20: note = DNA
  source 1..20: mol_type = other DNA
cataaatgccagtagagcaa

SEQ ID NO: 109 - DNA, 22 bp, synthetic construct
  misc_feature 1..22: note = THS for tcdC 117del Probe w/methoxy
  misc_feature 1..4: note = n is 2-O-Methyladenosine, DNA
  misc_feature 5: note = RNA
  misc_feature 6..22: note = DNA
  source 1..22: mol_type = other DNA
caantaaatgccagtagagcaa

SEQ ID NO: 110 - DNA, 22 bp, synthetic construct
  misc_feature 1..4: note = THS for tcdC 117del Probe w/methoxy, DNA
  misc_feature 5: note = RNA
  misc_feature 6..22: note = DNA
  modified_base 6: mod_base = OTHER; note = n is 2-O-Methyladenosine
  source 1..22: mol_type = other DNA
caaatnaatgccagtagagcaa

SEQ ID NO: 111 - DNA, 22 bp, synthetic construct
  misc_feature 1..22: note = THS for tcdC 117del Probe w/methoxy
  misc_feature 1..4: note = n is 2-O-Methyladenosine, DNA
  misc_feature 5: note = RNA
  misc_feature 6..22: note = DNA
  modified_base 6: mod_base = OTHER; note = n is 2-O-Methyladenosine
  source 1..22: mol_type = other DNA
caantnaatgccagtagagcaa

SEQ ID NO: 112 - DNA, 22 bp, synthetic construct
  misc_feature 1..22: note = THS for tcdC 117del Probe w/methoxy
  modified_base 2: mod_base = OTHER; note = n is 2-O-Methyladenosine
  source 1..22: mol_type = other DNA
cnaataaatgccagtagagcaa

SEQ ID NO: 113 - DNA, 23 bp, synthetic construct
  misc_feature 1..23: note = THS for tcdC 117del Probe w/methoxy
  modified_base 2: mod_base = OTHER; note = n is 2-O-Methyladenosine
  source 1..23: mol_type = other DNA
cnaaataaatgccagtagagcaa

SEQ ID NO: 114 - DNA, 22 bp, synthetic construct
  misc_feature 1..22: note = THS for tcdC 117del Probe w/methoxy
  misc_feature 1..4: note = n is 2-O-Methyladenosine, DNA
  misc_feature 5: note = RNA
  misc_feature 6..22: note = DNA
  source 1..22: mol_type = other DNA
cnaataaatgccagtagagcaa

SEQ ID NO: 115 - DNA, 23 bp, synthetic construct
  misc_feature 1..23: note = THS for tcdC 117del Probe w/methoxy
  misc_feature 1..5: note = n is 2-O-Methyladenosine, DNA
  misc_feature 6: note = RNA
  misc_feature 7..23: note = DNA
  source 1..23: mol_type = other DNA
cnaaataaatgccagtagagcaa

SEQ ID NO: 116 - DNA, 24 bp, synthetic construct
  misc_feature 1..4: note = THS for tcdC 117del Probe w/methoxy, DNA
  misc_feature 5: note = RNA
  misc_feature 6..24: note = DNA
  modified_base 6: mod_base = OTHER; note = n is 2-O-Methyladenosine
  source 1..24: mol_type = other DNA
caaatnaatgccagtagagcaata

SEQ ID NO: 117 - DNA, 25 bp, synthetic construct
  misc_feature 1..4: note = THS for tcdC 117del Probe w/methoxy, DNA
  misc_feature 5: note = RNA
  misc_feature 6..25: note = DNA
  modified_base 6: mod_base = OTHER; note = n is 2-O-Methyladenosine
  source 1..25: mol_type = other DNA
caaatnaatgccagtagagcaatac

SEQ ID NO: 118 - DNA, 26 bp, synthetic construct
  misc_feature 1..4: note = THS for tcdC 117del Probe w/methoxy, DNA
  misc_feature 5: note = RNA
  misc_feature 6..26: note = DNA
  modified_base 6: mod_base = OTHER; note = n is 2-O-Methyladenosine
  source 1..26: mol_type = other DNA
caaatnaatgccagtagagcaatacc

SEQ ID NO: 119 - DNA, 27 bp, synthetic construct
  misc_feature 1..4: note = THS for tcdC 117del Probe w/methoxy, DNA
  misc_feature 5: note = RNA
  misc_feature 6..27: note = DNA
  modified_base 6: mod_base = OTHER; note = n is 2-O-Methyladenosine
  source 1..27: mol_type = other DNA
caaatnaatgccagtagagcaataccc

SEQ ID NO: 120 - DNA, 24 bp, synthetic construct
  misc_feature 1..4: note = THS for tcdC 117del Probe w/methoxy, DNA
  misc_feature 5: note = RNA
  misc_feature 6..24: note = DNA
  modified_base 6: mod_base = OTHER; note = n is 2-O-Methyladenosine
  modified_base 11..12: mod_base = OTHER; note = n is 5-methylated cytosine
  modified_base 20: mod_base = OTHER; note = n is 5-methylated cytosine
  source 1..24: mol_type = other DNA
caaatnaatgnnagtagagnaata

SEQ ID NO: 121 - DNA, 24 bp, synthetic construct
  misc_feature 1..24: note = THS for tcdC 117del Probe w/methoxy
  misc_feature 1..4: note = n is 5-methylated cytosine, DNA
  misc_feature 5: note = RNA
  misc_feature 6..24: note = DNA
  modified_base 6: mod_base = OTHER; note = n is 2-O-Methyladenosine
  modified_base 11..12: mod_base = OTHER; note = n is 5-methylated cytosine
  modified_base 20: mod_base = OTHER; note = n is 5-methylated cytosine
  source 1..24: mol_type = other DNA
naaatnaatgnnagtagagnaata

SEQ ID NO: 122 - DNA, 24 bp, synthetic construct
  misc_feature 1..24: note = THS for tcdC 117del Probe w/methoxy
  misc_feature 1..4: note = n is 5-methylated cytosine, DNA
  misc_feature 5: note = RNA
  misc_feature 6..24: note = DNA
  modified_base 6: mod_base = OTHER; note = n is 2-O-Methyladenosine
  source 1..24: mol_type = other DNA
naaatnaatgccagtagagcaata

SEQ ID NO: 123 - DNA, 21 bp, synthetic construct
  misc_feature 1..21: note = THS for tcdC 117del Probe
  source 1..21: mol_type = other DNA
caaaataaatgccagtagagc

SEQ ID NO: 124 - DNA, 21 bp, synthetic construct
  misc_feature 1..21: note = THS for tcdC 117del Probe
  source 1..21: mol_type = other DNA
caaaataaatgccagtagagc

SEQ ID NO: 125 - DNA, 20 bp, synthetic construct
  misc_feature 1..20: note = THS for tcdC 117del Probe
  source 1..20: mol_type = other DNA
caaaataaatgccagtagag

SEQ ID NO: 126 - DNA, 19 bp, synthetic construct
  misc_feature 1..19: note = THS for tcdC 117del Probe
  source 1..19: mol_type = other DNA
caaaataaatgccagtaga

SEQ ID NO: 127 - DNA, 25 bp, synthetic construct
  misc_feature 1..25: note = THS for tcdC 117del Probe
  source 1..25: mol_type = other DNA
aaaataaatgccagtagagcaatac

SEQ ID NO: 128 - DNA, 25 bp, synthetic construct
  misc_feature 1..25: note = THS for tcdC 117del Probe
  source 1..25: mol_type = other DNA
aaaataaatgccagtagagcaatat

SEQ ID NO: 129 - DNA, 26 bp, synthetic construct
  misc_feature 1..26: note = THS for tcdC 117del Probe
  source 1..26: mol_type = other DNA
aaaataaatgccagtagagcaatatc

SEQ ID NO: 130 - DNA, 22 bp, synthetic construct
  misc_feature 1..22: note = THS for tcdC 117del Probe with abasic linker
  misc_feature 1^2: note = (abasic)-linker follows position 1
  source 1..22: mol_type = other DNA
caaataaatgccagtagagcaa

SEQ ID NO: 131 - DNA, 22 bp, synthetic construct
  misc_feature 1..22: note = THS for tcdC 117del Probe with abasic linker
  misc_feature 2^3: note = (abasic)-linker follows position 2
  source 1..22: mol_type = other DNA
caaataaatgccagtagagcaa

SEQ ID NO: 132 - DNA, 22 bp, synthetic construct
  misc_feature 1..22: note = THS for tcdC 117del Probe with C9 linker
  misc_feature 1^2: note = (sp9)-linker follows position 1
  source 1..22: mol_type = other DNA
caaataaatgccagtagagcaa

SEQ ID NO: 133 - DNA, 22 bp, synthetic construct
  misc_feature 1..22: note = THS for tcdC 117del Probe with C9 linker
  misc_feature 2^3: note = (sp9)-linker follows position 2
  source 1..22: mol_type = other DNA
caaataaatgccagtagagcaa

SEQ ID NO: 134 - DNA, 20 bp, synthetic construct
  misc_feature 1..20: note = THS for tcdC 117del Probe with abasic linker
  misc_feature 1^2: note = (abasic)-linker follows position 1
  source 1..20: mol_type = other DNA
caaataaatgccagtagagc

SEQ ID NO: 135 - DNA, 20 bp, synthetic construct
  misc_feature 1..20: note = THS for tcdC 117del Probe with abasic linker
  misc_feature 2^3: note = (abasic)-linker follows position 2
  source 1..20: mol_type = other DNA
caaataaatgccagtagagc

SEQ ID NO: 136 - DNA, 20 bp, synthetic construct
  misc_feature 1..20: note = THS for tcdC 117del Probe with C9 linker
  misc_feature 1^2: note = (sp9)-linker follows position 1
  source 1..20: mol_type = other DNA
caaataaatgccagtagagc

SEQ ID NO: 137 - DNA, 20 bp, synthetic construct
  misc_feature 1..20: note = THS for tcdC 117del Probe with C9 linker
  misc_feature 2^3: note = (sp9)-linker follows position 2
  source 1..20: mol_type = other DNA
caaataaatgccagtagagc

SEQ ID NO: 138 - DNA, 31 bp, synthetic construct
  misc_feature 1..31: note = tcdC 117del Primary Probe
  misc_feature 1..10: note = RNA
  misc_feature 11..31: note = DNA
  modified_base 31: mod_base = OTHER; note = 3'-terminal Hexanediol
  source 1..31: mol_type = other DNA
cgcgccgaggcaaaataaatgccagtagagc

SEQ ID NO: 139 - DNA, 35 bp, synthetic construct
  misc_feature 1..35: note = tcdC 117del Primary Probe
  misc_feature 1..10: note = RNA
  misc_feature 11..35: note = DNA
  modified_base 35: mod_base = OTHER; note = 3'-terminal Hexanediol
  source 1..35: mol_type = other DNA
cgcgccgaggaaaataaatgccagtagagcaatat

SEQ ID NO: 140 - DNA, 36 bp, synthetic construct
  misc_feature 1..36: note = tcdC 117del Primary Probe
  misc_feature 1..10: note = RNA
  misc_feature 11..36: note = DNA
  modified_base 36: mod_base = OTHER; note = 3'-terminal Hexanediol
  source 1..36: mol_type = other DNA
cgcgccgaggaaaataaatgccagtagagcaatatc

SEQ ID NO: 141 - DNA, 30 bp, synthetic construct
  misc_feature 1..30: note = tcdC 117del Primary Probe
  misc_feature 1..10: note = RNA
  misc_feature 11..30: note = DNA
  modified_base 30: mod_base = OTHER; note = 3'-terminal Hexanediol
  source 1..30: mol_type = other DNA
cgcgccgaggcaaaataaatgccagtagag

SEQ ID NO: 142 - DNA, 29 bp, synthetic construct
  misc_feature 1..29: note = tcdC 117del Primary Probe
  misc_feature 1..10: note = RNA
  misc_feature 11..29: note = DNA
  modified_base 29: mod_base = OTHER; note = 3'-terminal Hexanediol
  source 1..29: mol_type = other DNA
cgcgccgaggcaaaataaatgccagtaga

SEQ ID NO: 143 - DNA, 33 bp, synthetic construct
  misc_feature 1..33: note = tcdC 117del Primary Probe
  misc_feature 1..10: note = RNA
  misc_feature 11..33: note = DNA
  modified_base 33: mod_base = OTHER; note = 3'-terminal Hexanediol
  source 1..33: mol_type = other DNA
cgcgccgaggcaaaataaatgccagtagagcaa

SEQ ID NO: 144 - DNA, 36 bp, synthetic construct
  misc_feature 1..36: note = tcdC 117del Primary Probe
  misc_feature 1..10: note = RNA
  misc_feature 11..36: note = DNA
  modified_base 36: mod_base = OTHER; note = 3'-terminal Hexanediol
  source 1..36: mol_type = other DNA
cgcgccgaggcaaaataaatgccagtagagcaatac

SEQ ID NO: 145 - DNA, 32 bp, synthetic construct
  misc_feature 1..32: note = tcdC 117del Primary Probe
  misc_feature 1..10: note = RNA
  misc_feature 11..32: note = DNA
  modified_base 32: mod_base = OTHER; note = 3'-terminal Hexanediol
  source 1..32: mol_type = other DNA
cgcgccgaggcaaataaatgccagtagagcaa

SEQ ID NO: 146 - DNA, 31 bp, synthetic construct
  misc_feature 1..31: note = tcdC 117del Primary Probe
  misc_feature 1..10: note = RNA
  misc_feature 11..31: note = DNA
  modified_base 31: mod_base = OTHER; note = 3'-terminal Hexanediol
  source 1..31: mol_type = other DNA
cgcgccgaggcaataaatgccagtagagcaa

SEQ ID NO: 147 - DNA, 30 bp, synthetic construct
  misc_feature 1..30: note = tcdC 117del Primary Probe
  misc_feature 1..10: note = RNA
  misc_feature 11..30: note = DNA
  modified_base 30: mod_base = OTHER; note = 3'-terminal Hexanediol
  source 1..30: mol_type = other DNA
cgcgccgaggcataaatgccagtagagcaa

SEQ ID NO: 148 - DNA, 33 bp, synthetic construct; identical to SEQ ID NO: 143
  misc_feature 1..33: note = tcdC 117del Primary Probe w/methoxy
  misc_feature 1..10: note = RNA
  misc_feature 11..15: note = DNA
  misc_feature 16: note = RNA
  misc_feature 17..33: note = DNA
  modified_base 33: mod_base = OTHER; note = 3'-terminal Hexanediol
  source 1..33: mol_type = other DNA

SEQ ID NO: 149 - DNA, 33 bp, synthetic construct
  misc_feature 1..33: note = tcdC 117del Primary Probe w/methoxy
  misc_feature 1..10: note = RNA
  misc_feature 11..33: note = DNA
  modified_base 14: mod_base = OTHER; note = n is 2-O-Methyladenosine
  modified_base 33: mod_base = OTHER; note = 3'-terminal Hexanediol
  source 1..33: mol_type = other DNA
cgcgccgaggcaanataaatgccagtagagcaa

SEQ ID NO: 150 - DNA, 33 bp, synthetic construct
  misc_feature 1..33: note = tcdC 117del Primary Probe w/methoxy
  misc_feature 1..10: note = RNA
  misc_feature 11..33: note = DNA
  modified_base 13: mod_base = OTHER; note = n is 2-O-Methyladenosine
  modified_base 33: mod_base = OTHER; note = 3'-terminal Hexanediol
  source 1..33: mol_type = other DNA
cgcgccgaggcanaataaatgccagtagagcaa

SEQ ID NO: 151 - DNA, 32 bp, synthetic construct; identical to SEQ ID NO: 145
  misc_feature 1..32: note = tcdC 117del Primary Probe w/methoxy
  misc_feature 1..10: note = RNA
  misc_feature 11..14: note = DNA
  misc_feature 15: note = RNA
  misc_feature 16..32: note = DNA
  modified_base 32: mod_base = OTHER; note = 3'-terminal Hexanediol
  source 1..32: mol_type = other DNA

SEQ ID NO: 152 - DNA, 31 bp, synthetic construct
  misc_feature 1..31: note = tcdC 117del Primary Probe w/methoxy
  misc_feature 1..10: note = RNA
  misc_feature 11..31: note = DNA
  modified_base 14: mod_base = OTHER; note = n is U
  modified_base 31: mod_base = OTHER; note = 3'-terminal Hexanediol
  source 1..31: mol_type = other DNA
cgcgccgaggcaanaaatgccagtagagcaa

SEQ ID NO: 153 - DNA, 30 bp, synthetic construct
  misc_feature 1..30: note = tcdC 117del Primary Probe w/methoxy
  misc_feature 1..10: note = RNA
  misc_feature 11..30: note = DNA
  modified_base 13: mod_base = OTHER; note = n is U
  modified_base 30: mod_base = OTHER; note = 3'-terminal Hexanediol
  source 1..30: mol_type = other DNA
cgcgccgaggcanaaatgccagtagagcaa

SEQ ID NO: 154 - DNA, 32 bp, synthetic construct
  misc_feature 1..32: note = tcdC 117del Primary Probe w/methoxy
  misc_feature 1..10: note = RNA
  misc_feature 11..32: note = DNA
  modified_base 14: mod_base = OTHER; note = n is 2-O-Methyladenosine
  modified_base 15: mod_base = OTHER; note = n is U
  modified_base 32: mod_base = OTHER; note = 3'-terminal Hexanediol
  source 1..32: mol_type = other DNA
cgcgccgaggcaannaaatgccagtagagcaa

SEQ ID NO: 155 - DNA, 32 bp, synthetic construct
  misc_feature 1..32: note = tcdC 117del Primary Probe w/methoxy
  misc_feature 1..10: note = RNA
  misc_feature 11..32: note = DNA
  modified_base 14: mod_base = OTHER; note = n is U
  modified_base 15: mod_base = OTHER; note = n is 2-O-Methyladenosine
  misc_feature 16: note = variant - n is a, c, g, or t
  modified_base 32: mod_base = OTHER; note = 3'-terminal Hexanediol
  source 1..32: mol_type = other DNA
cgcgccgaggcaaannaatgccagtagagcaa

SEQ ID NO: 156 - DNA, 32 bp, synthetic construct
  misc_feature 1..32: note = tcdC 117del Primary Probe w/methoxy
  misc_feature 1..10: note = RNA
  misc_feature 11..32: note = DNA
  modified_base 14: mod_base = OTHER; note = n is 2-O-Methyladenosine
  modified_base 15: mod_base = OTHER; note = n is U
  modified_base 16: mod_base = OTHER; note = n is 2-O-Methyladenosine
  modified_base 32: mod_base = OTHER; note = 3'-terminal Hexanediol
  source 1..32: mol_type = other DNA
cgcgccgaggcaannnaatgccagtagagcaa

SEQ ID NO: 157 - DNA, 32 bp, synthetic construct
  misc_feature 1..32: note = tcdC 117del Primary Probe w/methoxy
  misc_feature 1..10: note = RNA
  misc_feature 11..32: note = DNA
  modified_base 12: mod_base = OTHER; note = n is 2-O-Methyladenosine
  modified_base 32: mod_base = OTHER; note = 3'-terminal Hexanediol
  source 1..32: mol_type = other DNA
cgcgccgaggcnaataaatgccagtagagcaa

SEQ ID NO: 158 - DNA, 33 bp, synthetic construct
  misc_feature 1..33: note = tcdC 117del Primary Probe w/methoxy
  misc_feature 1..10: note = RNA
  misc_feature 11..33: note = DNA
  modified_base 12: mod_base = OTHER; note = n is 2-O-Methyladenosine
  modified_base 33: mod_base = OTHER; note = 3'-terminal Hexanediol
  source 1..33: mol_type = other DNA
cgcgccgaggcnaaataaatgccagtagagcaa

SEQ ID NO: 159 - DNA, 32 bp, synthetic construct
  misc_feature 1..32: note = tcdC 117del Primary Probe w/methoxy
  misc_feature 1..10: note = RNA
  misc_feature 11..32: note = DNA
  modified_base 12: mod_base = OTHER; note = n is 2-O-Methyladenosine
  modified_base 15: mod_base = OTHER; note = n is U
  modified_base 32: mod_base = OTHER; note = 3'-terminal Hexanediol
  source 1..32: mol_type = other DNA
cgcgccgaggcnaanaaatgccagtagagcaa

SEQ ID NO: 160 - DNA, 33 bp, synthetic construct
  misc_feature 1..33: note = tcdC 117del Primary Probe w/methoxy
  misc_feature 1..10: note = RNA
  misc_feature 11..33: note = DNA
  modified_base 12: mod_base = OTHER; note = n is 2-O-Methyladenosine
  modified_base 16: mod_base = OTHER; note = n is U
  modified_base 33: mod_base = OTHER; note = 3'-terminal Hexanediol
  source 1..33: mol_type = other DNA
cgcgccgaggcnaaanaaatgccagtagagcaa

SEQ ID NO: 161 - DNA, 34 bp, synthetic construct
  misc_feature 1..34: note = tcdC 117del Primary Probe w/methoxy
  misc_feature 1..10: note = RNA
  misc_feature 11..34: note = DNA
  modified_base 15: mod_base = OTHER; note = n is U
  modified_base 16: mod_base = OTHER; note = n is 2-O-Methyladenosine
  modified_base 34: mod_base = OTHER; note = 3'-terminal Hexanediol
  source 1..34: mol_type = other DNA
cgcgccgaggcaaannaatgccagtagagcaata

SEQ ID NO: 162 - DNA, 35 bp, synthetic construct
  misc_feature 1..35: note = tcdC 117del Primary Probe w/methoxy
  misc_feature 1..10: note = RNA
  misc_feature 11..35: note = DNA
  modified_base 15: mod_base = OTHER; note = n is U
  modified_base 16: mod_base = OTHER; note = n is 2-O-Methyladenosine
  modified_base 35: mod_base = OTHER; note = 3'-terminal Hexanediol
  source 1..35: mol_type = other DNA
cgcgccgaggcaaannaatgccagtagagcaatac

SEQ ID NO: 163 - DNA, 36 bp, synthetic construct
  misc_feature 1..36: note = tcdC 117del Primary Probe w/methoxy
  misc_feature 1..10: note = RNA
  misc_feature 11..36: note = DNA
  modified_base 15: mod_base = OTHER; note = n is U
  modified_base 16: mod_base = OTHER; note = n is 2-O-Methyladenosine
  modified_base 36: mod_base = OTHER; note = 3'-terminal Hexanediol
  source 1..36: mol_type = other DNA
cgcgccgaggcaaannaatgccagtagagcaatacc

SEQ ID NO: 164 - DNA, 37 bp, synthetic construct
  misc_feature 1..37: note = tcdC 117del Primary Probe w/methoxy
  misc_feature 1..10: note = RNA
  misc_feature 11..37: note = DNA
  modified_base 15: mod_base = OTHER; note = n is U
  modified_base 16: mod_base = OTHER; note = n is 2-O-Methyladenosine
  modified_base 37: mod_base = OTHER; note = 3'-terminal Hexanediol
  source 1..37: mol_type = other DNA
cgcgccgaggcaaannaatgccagtagagcaataccc

SEQ ID NO: 165 - DNA, 34 bp, synthetic construct
  misc_feature 1..34: note = tcdC 117del Primary Probe w/methoxy
  misc_feature 1..10: note = RNA
  misc_feature 11..34: note = DNA
  modified_base 15: mod_base = OTHER; note = n is U
  modified_base 16: mod_base = OTHER; note = n is 2-O-Methyladenosine
  modified_base 21..22: mod_base = OTHER; note = n is 5-methylated cytosine
  modified_base 30: mod_base = OTHER; note = n is 5-methylated cytosine
  modified_base 34: mod_base = OTHER; note = 3'-terminal Hexanediol
  source 1..34: mol_type = other DNA
cgcgccgaggcaaannaatgnnagtagagnaata

SEQ ID NO: 166 - DNA, 34 bp, synthetic construct
  misc_feature 1..34: note = tcdC 117del Primary Probe w/methoxy
  misc_feature 1..10: note = RNA
  misc_feature 11..34: note = DNA
  modified_base 11: mod_base = OTHER; note = n is 5-methylated cytosine
  modified_base 15: mod_base = OTHER; note = n is U
  modified_base 16: mod_base = OTHER; note = n is 2-O-Methyladenosine
  modified_base 21..22: mod_base = OTHER; note = n is 5-methylated cytosine
  modified_base 30: mod_base = OTHER; note = n is 5-methylated cytosine
  modified_base 34: mod_base = OTHER; note = 3'-terminal Hexanediol
  source 1..34: mol_type = other DNA
cgcgccgaggnaaannaatgnnagtagagnaata

SEQ ID NO: 167 - DNA, 34 bp, synthetic construct
  misc_feature 1..34: note = tcdC 117del Primary Probe w/methoxy
  misc_feature 1..10: note = RNA
  misc_feature 11..34: note = DNA
  modified_base 11: mod_base = OTHER; note = n is 5-methylated cytosine
  modified_base 15: mod_base = OTHER; note = n is U
  modified_base 16: mod_base = OTHER; note = n is 2-O-Methyladenosine
  modified_base 34: mod_base = OTHER; note = 3'-terminal Hexanediol
  source 1..34: mol_type = other DNA
cgcgccgaggnaaannaatgccagtagagcaata

SEQ ID NO: 168 - DNA, 35 bp, synthetic construct
  misc_feature 1..35: note = tcdC 117del Primary Probe
  misc_feature 1..14: note = RNA
  misc_feature 15..35: note = DNA
  modified_base 35: mod_base = OTHER; note = 3'-terminal Hexanediol
  source 1..35: mol_type = other DNA
atgacgtggcagaccaaaataaatgccagtagagc

SEQ ID NO: 169 - DNA, 33 bp, synthetic construct
  misc_feature 1..33: note = tcdC 117del Primary Probe
  misc_feature 1..12: note = RNA
  misc_feature 13..33: note = DNA
  modified_base 33: mod_base = OTHER; note = 3'-terminal Hexanediol
  source 1..33: mol_type = other DNA
acggacgcggagcaaaataaatgccagtagagc

SEQ ID NO: 170 - DNA, 32 bp, synthetic construct
  misc_feature 1..32: note = tcdC 117del Primary Probe
  misc_feature 1..12: note = RNA
  misc_feature 13..32: note = DNA
  modified_base 32: mod_base = OTHER; note = 3'-terminal Hexanediol
  source 1..32: mol_type = other DNA
acggacgcggagcaaaataaatgccagtagag

SEQ ID NO: 171 - DNA, 31 bp, synthetic construct
  misc_feature 1..31: note = tcdC 117del Primary Probe
  misc_feature 1..12: note = RNA
  misc_feature 13..31: note = DNA
  modified_base 31: mod_base = OTHER; note = 3'-terminal Hexanediol
  source 1..31: mol_type = other DNA
acggacgcggagcaaaataaatgccagtaga

SEQ ID NO: 172 - DNA, 37 bp, synthetic construct
  misc_feature 1..37: note = tcdC 117del Primary Probe
  misc_feature 1..12: note = RNA
  misc_feature 13..37: note = DNA
  modified_base 37: mod_base = OTHER; note = 3'-terminal Hexanediol
  source 1..37: mol_type = other DNA
acggacgcggagaaaataaatgccagtagagcaatac

SEQ ID NO: 173 - DNA, 37 bp, synthetic construct
  misc_feature 1..37: note = tcdC 117del Primary Probe
  misc_feature 1..12: note = RNA
  misc_feature 13..37: note = DNA
  modified_base 37: mod_base = OTHER; note = 3'-terminal Hexanediol
  source 1..37: mol_type = other DNA
acggacgcggagaaaataaatgccagtagagcaatat

SEQ ID NO: 174 - DNA, 38 bp, synthetic construct
  misc_feature 1..38: note = tcdC 117del Primary Probe
  misc_feature 1..12: note = RNA
  misc_feature 13..38: note = DNA
  modified_base 38: mod_base = OTHER; note = 3'-terminal Hexanediol
  source 1..38: mol_type = other DNA
acggacgcggagaaaataaatgccagtagagcaatatc

SEQ ID NO: 175 - DNA, 34 bp, synthetic construct
  misc_feature 1..34: note = tcdC 117del Primary Probe with abasic linker
  misc_feature 1..12: note = RNA
  misc_feature 13..34: note = DNA
  misc_feature 13^14: note = (abasic)-linker follows position 13
  modified_base 34: mod_base = OTHER; note = 3'-terminal Hexanediol
  source 1..34: mol_type = other DNA
acggacgcggagcaaataaatgccagtagagcaa

SEQ ID NO: 176 - DNA, 34 bp, synthetic construct; identical to SEQ ID NO: 175
  misc_feature 1..34: note = tcdC 117del Primary Probe with abasic linker
  misc_feature 1..12: note = RNA
  misc_feature 13..34: note = DNA
  misc_feature 14^15: note = (abasic)-linker follows position 14
  modified_base 34: mod_base = OTHER; note = 3'-terminal Hexanediol
  source 1..34: mol_type = other DNA

SEQ ID NO: 177 - DNA, 34 bp, synthetic construct; identical to SEQ ID NO: 175
  misc_feature 1..34: note = tcdC 117del Primary Probe with C9 linker
  misc_feature 1..12: note = RNA
  misc_feature 13..34: note = DNA
  misc_feature 13^14: note = (sp9)-linker follows position 13
  modified_base 34: mod_base = OTHER; note = 3'-terminal Hexanediol
  source 1..34: mol_type = other DNA

SEQ ID NO: 178 - DNA, 34 bp, synthetic construct; identical to SEQ ID NO: 175
  misc_feature 1..34: note = tcdC 117del Primary Probe with C9 linker
  misc_feature 1..12: note = RNA
  misc_feature 13..34: note = DNA
  misc_feature 14^15: note = (sp9)-linker follows position 14
  modified_base 34: mod_base = OTHER; note = 3'-terminal Hexanediol
  source 1..34: mol_type = other DNA

SEQ ID NO: 179 - DNA, 32 bp, synthetic construct
  misc_feature 1..32: note = tcdC 117del Primary Probe with abasic linker
  misc_feature 1..12: note = RNA
  misc_feature 13..32: note = DNA
  misc_feature 13^14: note = (abasic)-linker follows position 13
  modified_base 32: mod_base = OTHER; note = 3'-terminal Hexanediol
  source 1..32: mol_type = other DNA
acggacgcggagcaaataaatgccagtagagc

SEQ ID NO: 180 - DNA, 32 bp, synthetic construct; identical to SEQ ID NO: 179
  misc_feature 1..32: note = tcdC 117del Primary Probe with abasic linker
  misc_feature 1..12: note = RNA
  misc_feature 13..32: note = DNA
  misc_feature 14^15: note = (abasic)-linker follows position 14
  modified_base 32: mod_base = OTHER; note = 3'-terminal Hexanediol
  source 1..32: mol_type = other DNA

SEQ ID NO: 181 - DNA, 32 bp, synthetic construct; identical to SEQ ID NO: 179
  misc_feature 1..32: note = tcdC 117del Primary Probe with C9 linker
  misc_feature 1..12: note = RNA
  misc_feature 13..32: note = DNA
  misc_feature 13^14: note = (sp9)-linker follows position 13
  modified_base 32: mod_base = OTHER; note = 3'-terminal Hexanediol
  source 1..32: mol_type = other DNA

SEQ ID NO: 182 - DNA, 32 bp, synthetic construct; identical to SEQ ID NO: 179
  misc_feature 1..32: note = tcdC 117del Primary Probe with C9 linker
  misc_feature 1..12: note = RNA
  misc_feature 13..32: note = DNA
  misc_feature 14^15: note = (sp9)-linker follows position 14
  modified_base 32: mod_base = OTHER; note = 3'-terminal Hexanediol
  source 1..32: mol_type = other DNA

SEQ ID NO: 183 - DNA, 32 bp, synthetic construct
  misc_feature 1..32: note = tcdC 117del Invader
  misc_feature 1..31: note = DNA
  misc_feature 32: note = RNA
  source 1..32: mol_type = other DNA
acaaagggtattgctctactggcatttatttc

SEQ ID NO: 184 - DNA, 21 bp, synthetic construct
  misc_feature 1..21: note = THS for tcdC 117del Primary Probe
  misc_feature 1..3: note = DNA
  misc_feature 4: note = RNA
  misc_feature 5..21: note = DNA
  source 1..21: mol_type = other DNA
tggtgtgttttttggcaatat

SEQ ID NO: 185 - DNA, 23 bp, synthetic construct
  misc_feature 1..23: note = THS for tcdC 117del Primary Probe
  misc_feature 1..2: note = DNA
  misc_feature 3: note = RNA
  misc_feature 4..23: note = DNA
  source 1..23: mol_type = other DNA
ggtgtgttttttggcaatatatc

SEQ ID NO: 186 - DNA, 20 bp, synthetic construct
  misc_feature 1..20: note = THS for tcdC 117del Primary Probe
  source 1..20: mol_type = other DNA
ggtgtgttttttggcaatat

SEQ ID NO: 187 - DNA, 23 bp, synthetic construct
  misc_feature 1..23: note = THS for tcdC 117del Primary Probe
  source 1..23: mol_type = other DNA
ggtgtgttttttggcaatatatc

SEQ ID NO: 188 - DNA, 33 bp, synthetic construct
  misc_feature 1..33: note = tcdC 117del Primary Probe
  misc_feature 1..12: note = RNA
  misc_feature 13..15: note = DNA
  misc_feature 16: note = RNA
  misc_feature 17..33: note = DNA
  modified_base 33: mod_base = OTHER; note = 3'-terminal Hexanediol
  source 1..33: mol_type = other DNA
acggacgcggagtggtgtgttttttggcaatat

SEQ ID NO: 189 - DNA, 37 bp, synthetic construct
  misc_feature 1..37: note = tcdC 117del Primary Probe
  misc_feature 1..14: note = RNA
  misc_feature 15..16: note = DNA
  misc_feature 17: note = RNA
  misc_feature 18..37: note = DNA
  modified_base 37: mod_base = OTHER; note = 3'-terminal Hexanediol
  source 1..37: mol_type = other DNA
atgacgtggcagacggtgtgttttttggcaatatatc

SEQ ID NO: 190 - DNA, 34 bp, synthetic construct
  misc_feature 1..34: note = tcdC 117del Primary Probe
  misc_feature 1..14: note = RNA
  misc_feature 15..16: note = DNA
  misc_feature 17: note = RNA
  misc_feature 18..34: note = DNA
  modified_base 34: mod_base = OTHER; note = 3'-terminal Hexanediol
  source 1..34: mol_type = other DNA
atgacgtggcagacggtgtgttttttggcaatat

SEQ ID NO: 191 - DNA, 35 bp, synthetic construct
  misc_feature 1..35: note = tcdC 117del Primary Probe
  misc_feature 1..12: note = RNA
  misc_feature 13..14: note = DNA
  misc_feature 15: note = RNA
  misc_feature 16..35: note = DNA
  modified_base 35: mod_base = OTHER; note = 3'-terminal Hexanediol
  source 1..35: mol_type = other DNA
acggacgcggagggtgtgttttttggcaatatatc

SEQ ID NO: 192 - DNA, 29 bp, synthetic construct
  misc_feature 1..29: note = tcdC 117del Reverse iPrimer
  source 1..29: mol_type = other DNA
ggtgaggatatattgccaaaaaacacgcc

SEQ ID NO: 193 - DNA, 30 bp, synthetic construct
  misc_feature 1..30: note = tcdC 117del Reverse iPrimer
  source 1..30: mol_type = other DNA
ggtgaggatatattgccaaaaaacacgcca

SEQ ID NO: 194 - DNA, 30 bp, synthetic construct
  misc_feature 1..30: note = tcdC 183/184 Invader
  misc_feature 1..29: note = DNA
  misc_feature 30: note = RNA
  source 1..30: mol_type = other DNA
ctgaagaccatgaggaggtcatttctaata

SEQ ID NO: 195 - DNA, 32 bp, synthetic construct
  misc_feature 1..32: note = tcdC 183/184 Invader
  source 1..32: mol_type = other DNA
gttctgaagaccatgaggaggtcatttctaag

SEQ ID NO: 196 - DNA, 33 bp, synthetic construct
  misc_feature 1..33: note = tcdC 183/184 Invader
  source 1..33: mol_type = other DNA
gttctgaagaccatgaggaggtcatttctaatg

SEQ ID NO: 197 - DNA, 23 bp, synthetic construct
  misc_feature 1..23: note = THS for tcdC 183/184 CC>TT Primary Probe
  source 1..23: mol_type = other DNA
ttaaacatcagttatagattctc

SEQ ID NO: 198 - DNA, 22 bp, synthetic construct
  misc_feature 1..22: note = THS for tcdC 183/184 CC>TT Primary Probe
  source 1..22: mol_type = other DNA
taaacatcagttatagattctc

SEQ ID NO: 199 - DNA, 23 bp, synthetic construct
  misc_feature 1..23: note = THS for tcdC 183/184 CC>TT Primary Probe
  source 1..23: mol_type = other DNA
ttaaacatcagttatagattctc

SEQ ID NO: 200 - DNA, 22 bp, synthetic construct
  misc_feature 1..22: note = THS for tcdC 183/184 CC>TT Primary Probe
  modified_base 5: mod_base = OTHER; note = n is 5-methylated cytosine
  source 1..22: mol_type = other DNA
taaanatcagttatagattctc

SEQ ID NO: 201 - DNA, 33 bp, synthetic construct
  misc_feature 1..33: note = tcdC 183/184 CC>TT Primary Probe
  misc_feature 1..10: note = RNA
  misc_feature 11..33: note = DNA
  modified_base 33: mod_base = OTHER; note = 3'-terminal Hexanediol
  source 1..33: mol_type = other DNA
cgcgccgaggttaaacatcagttatagattctc

SEQ ID NO: 202 - DNA, 32 bp, synthetic construct
  misc_feature 1..32: note = tcdC 183/184 CC>TT Primary Probe
  misc_feature 1..10: note = RNA
  misc_feature 11..32: note = DNA
  modified_base 32: mod_base = OTHER; note = 3'-terminal Hexanediol
  source 1..32: mol_type = other DNA
cgcgccgaggtaaacatcagttatagattctc

SEQ ID NO: 203 - DNA, 33 bp, synthetic construct; identical to SEQ ID NO: 201
  misc_feature 1..33: note = tcdC 183/184 CC>TT Primary Probe
  misc_feature 1..10: note = RNA
  misc_feature 11..33: note = DNA
  modified_base 33: mod_base = OTHER; note = 3'-terminal Hexanediol
  source 1..33: mol_type = other DNA

SEQ ID NO: 204 - DNA, 32 bp, synthetic construct
  misc_feature 1..32: note = tcdC 183/184 CC>TT Primary Probe
  misc_feature 1..10: note = RNA
  misc_feature 11..32: note = DNA
  modified_base 15: mod_base = OTHER; note = n is 5-methylated cytosine
  modified_base 32: mod_base = OTHER; note = 3'-terminal Hexanediol
  source 1..32: mol_type = other DNA
cgcgccgaggtaaanatcagttatagattctc

SEQ ID NO: 205 - DNA, 41 bp, synthetic construct
  misc_feature 1..41: note = tcdC 184 Invader
  misc_feature 1..40: note = DNA
  misc_feature 41: note = RNA
  source 1..41: mol_type = other DNA
gtttctatttctgttttttgagaatctataactgatgtttt

SEQ ID NO: 206 - DNA, 23 bp, synthetic construct
  misc_feature 1..23: note = THS for tcdC 183C>T 184C>T Primary Probe
  source 1..23: mol_type = other DNA
aattagaaatgacctcctcatgg

SEQ ID NO: 207 - DNA, 23 bp, synthetic construct
  misc_feature 1..23: note = THS for tcdC 183C>T 184C>T Primary Probe
  source 1..23: mol_type = other DNA
attagaaatgacctcctcatggt

SEQ ID NO: 208 - DNA, 19 bp, synthetic construct
  misc_feature 1..19: note = THS for tcdC 183C>T 184C>T Primary Probe
  source 1..19: mol_type = other DNA
aattagaaatgacctcctc

SEQ ID NO: 209 - DNA, 18 bp, synthetic construct
  misc_feature 1..18: note = THS for tcdC 183C>T 184C>T Primary Probe
  source 1..18: mol_type = other DNA
attagaaatgacctcctc

SEQ ID NO: 210 - DNA, 23 bp, synthetic construct
  misc_feature 1..23: note = THS for tcdC 183C>T 184C>T Primary Probe
  source 1..23: mol_type = other DNA
aattagaaatgacctcctcatgg

SEQ ID NO: 211 - DNA, 23 bp, synthetic construct
  misc_feature 1..23: note = THS for tcdC 183C>T 184C>T Primary Probe
  source 1..23: mol_type = other DNA
aattagaaatgacctcctcatgg

SEQ ID NO: 212 - DNA, 23 bp, synthetic construct
  misc_feature 1..23: note = THS for tcdC 183C>T 184C>T Primary Probe
  source 1..23: mol_type = other DNA
attagaaatgacctcctcatggt

SEQ ID NO: 213 - DNA, 33 bp, synthetic construct
  misc_feature 1..33: note = tcdC 183C>T 184C>T Primary Probe
  misc_feature 1..10: note = RNA
  misc_feature 11..33: note = DNA
  modified_base 33: mod_base = OTHER; note = 3'-terminal Hexanediol
  source 1..33: mol_type = other DNA
cgcgccgaggaattagaaatgacctcctcatgg

SEQ ID NO: 214 - DNA, 33 bp, synthetic construct
  misc_feature 1..33: note = tcdC 183C>T 184C>T Primary Probe
  misc_feature 1..10: note = RNA
  misc_feature 11..33: note = DNA
  modified_base 33: mod_base = OTHER; note = 3'-terminal Hexanediol
  source 1..33: mol_type = other DNA
cgcgccgaggattagaaatgacctcctcatggt

SEQ ID NO: 215 - DNA, 29 bp, synthetic construct
  misc_feature 1..29: note = tcdC 183C>T 184C>T Primary Probe
  misc_feature 1..10: note = RNA
  misc_feature 11..29: note = DNA
  modified_base 29: mod_base = OTHER; note = 3'-terminal Hexanediol
  source 1..29: mol_type = other DNA
cgcgccgaggaattagaaatgacctcctc

SEQ ID NO: 216 - DNA, 28 bp, synthetic construct
  misc_feature 1..28: note = tcdC 183C>T 184C>T Primary Probe
  misc_feature 1..10: note = RNA
  misc_feature 11..28: note = DNA
  modified_base 28: mod_base = OTHER; note = 3'-terminal Hexanediol
  source 1..28: mol_type = other DNA
cgcgccgaggattagaaatgacctcctc

SEQ ID NO: 217 - DNA, 37 bp, synthetic construct
  misc_feature 1..37: note = tcdC 183C>T 184C>T Primary Probe
  misc_feature 1..14: note = RNA
  misc_feature 15..37: note = DNA
  modified_base 37: mod_base = OTHER; note = 3'-terminal Hexanediol
  source 1..37: mol_type = other DNA
atgacgtggcagacaattagaaatgacctcctcatgg

SEQ ID NO: 218 - DNA, 35 bp, synthetic construct
  misc_feature 1..35: note = tcdC 183C>T 184C>T Primary Probe
  misc_feature 1..12: note = RNA
  misc_feature 13..35: note = DNA
  modified_base 35: mod_base = OTHER; note = 3'-terminal Hexanediol
  source 1..35: mol_type = other DNA
acggacgcggagaattagaaatgacctcctcatgg

SEQ ID NO: 219 - DNA, 35 bp, synthetic construct
  misc_feature 1..35: note = tcdC 183C>T 184C>T Primary Probe
  misc_feature 1..12: note = RNA
  misc_feature 13..35: note = DNA
  modified_base 35: mod_base = OTHER; note = 3'-terminal Hexanediol
  source 1..35: mol_type = other DNA
acggacgcggagattagaaatgacctcctcatggt

SEQ ID NO: 220 - DNA, 35 bp, synthetic construct
  misc_feature 1..35: note = tcdC 183T Forward iPrimer (+)
  source 1..35: mol_type = other DNA
cttgttctgaagaccatgaggaggtcatttctaat

SEQ ID NO: 221 - DNA, 42 bp, synthetic construct
  misc_feature 1..42: note = tcdC 184 Forward iPrimer (-)
  source 1..42: mol_type = other DNA
gtttctatttctgttttttgagaatctataactgatgtttaa

SEQ ID NO: 222 - DNA, 30 bp, synthetic construct
  misc_feature 1..30: note = tcdC 184 Forward Primer
  source 1..30: mol_type = other DNA
gcaatatatcctcaccagcttgttctgaag

SEQ ID NO: 223 - DNA, 28 bp, synthetic construct
  misc_feature 1..28: note = tcdC 184 Invader
  source 1..28: mol_type = other DNA
gaagaccatgaggaggtcatttctaaca

SEQ ID NO: 224 - skipped

SEQ ID NO: 225 - DNA, 41 bp, synthetic construct
  misc_feature 1..41: note = tcdC 184 Iprimer 2 (-)
  source 1..41: mol_type = other DNA
gtttctatttctgttttttgagaatctataactgatgttta

SEQ ID NO: 226-232 - skipped (7 entries)

SEQ ID NO: 233 - DNA, 22 bp, synthetic construct
  misc_feature 1..22: note = THS for tcdC 184C>T Primary Probe
  source 1..22: mol_type = other DNA
agttagaaatgacctcctcatg

SEQ ID NO: 234 - DNA, 29 bp, synthetic construct
  misc_feature 1..29: note = THS for tcdC 184C>T Primary Probe
  source 1..29: mol_type = other DNA
taaacatcagttatagattctcaaaaagc

SEQ ID NO: 235 - DNA, 29 bp, synthetic construct
  misc_feature 1..29: note = THS for tcdC 184C>T Primary Probe
  source 1..29: mol_type = other DNA
taaacatcagttatagattctcaaaaagc

SEQ ID NO: 236 - DNA, 22 bp, synthetic construct
  misc_feature 1..22: note = THS for tcdC 184C>T Primary Probe
  source 1..22: mol_type = other DNA
agttagaaatgacctcctcatg

SEQ ID NO: 237 - DNA, 22 bp, synthetic construct
  misc_feature 1..22: note = THS for tcdC 184C>T Primary Probe
  source 1..22: mol_type = other DNA
taaacatcagttatagattctc

SEQ ID NO: 238 - DNA, 36 bp, synthetic construct
  misc_feature 1..36: note = tcdC 184C>T Primary Probe
  misc_feature 1..14: note = RNA
  misc_feature 15..36: note = DNA
  modified_base 36: mod_base = OTHER; note = 3'-terminal Hexanediol
  source 1..36: mol_type = other DNA
atgacgtggcagacagttagaaatgacctcctcatg

SEQ ID NO: 239 - DNA, 43 bp, synthetic construct
  misc_feature 1..43: note = tcdC 184C>T Primary Probe
  misc_feature 1..14: note = RNA
  misc_feature 15..43: note = DNA
  modified_base 43: mod_base = OTHER; note = 3'-terminal Hexanediol
  source 1..43: mol_type = other DNA
atgacgtggcagactaaacatcagttatagattctcaaaaagc

SEQ ID NO: 240 - DNA, 41 bp, synthetic construct
  misc_feature 1..41: note = tcdC 184C>T Primary Probe
  misc_feature 1..12: note = RNA
  misc_feature 13..41: note = DNA
  modified_base 41: mod_base = OTHER; note = 3'-terminal Hexanediol
  source 1..41: mol_type = other DNA
acggacgcggagtaaacatcagttatagattctcaaaaagc

SEQ ID NO: 241 - DNA, 34 bp, synthetic construct
  misc_feature 1..34: note = tcdC 184C>T Primary Probe
  misc_feature 1..12: note = RNA
  misc_feature 13..34: note = DNA
  modified_base 34: mod_base = OTHER; note = 3'-terminal Hexanediol
  source 1..34: mol_type = other DNA
acggacgcggagagttagaaatgacctcctcatg

SEQ ID NO: 242 - DNA, 34 bp, synthetic construct
  misc_feature 1..34: note = tcdC 184C>T Primary Probe
  misc_feature 1..12: note = RNA
  misc_feature 13..34: note = DNA
  modified_base 34: mod_base = OTHER; note = 3'-terminal Hexanediol
  source 1..34: mol_type = other DNA
acggacgcggagtaaacatcagttatagattctc

SEQ ID NO: 243 - DNA, 42 bp, synthetic construct
  misc_feature 1..42: note = tcdC Forward Primer
  source 1..42: mol_type = other DNA
ggaaaaggaagctctaagaaaataattaaattctttaagagc

SEQ ID NO: 244 - DNA, 44 bp, synthetic construct
  misc_feature 1..44: note = tcdC Forward Primer
  source 1..44: mol_type = other DNA
ggaagctctaagaaaataattaaattctttaagagcacaaaggg

SEQ ID NO: 245 - DNA, 21 bp, synthetic construct
  misc_feature 1..21: note = tcdC Forward Primer
  modified_base 6: mod_base = OTHER; note = n is 5-methylated cytosine
  source 1..21: mol_type = other DNA
ggtaangaatttagtaatgaa

SEQ ID NO: 246 - DNA, 21 bp, synthetic construct
  misc_feature 1..21: note = tcdC Forward Primer
  modified_base 6: mod_base = OTHER; note = n is 5-methylated cytosine
  source 1..21: mol_type = other DNA
ggtaangaatttagtaatgaa

SEQ ID NO: 247 - DNA, 43 bp, synthetic construct
  misc_feature 1..43: note = tcdC Forward Primer
  source 1..43: mol_type = other DNA
ggaagctctaagaaaataattaaattctttaagagcacaaagg

SEQ ID NO: 248 - DNA, 33 bp, synthetic construct
  misc_feature 1..33: note = tcdC Forward Primer
  source 1..33: mol_type = other DNA
ggtaacgaatttagtaatgaaggaaaaggaagc

SEQ ID NO: 249 - DNA, 40 bp, synthetic construct
  misc_feature 1..40: note = tcdC Reverse Primer
  source 1..40: mol_type = other DNA
gttcagcatcagacaatttgctatttaaagtttctatttc

SEQ ID NO: 250 - DNA, 29 bp, synthetic construct
  misc_feature 1..29: note = tcdC Reverse Primer
  source 1..29: mol_type = other DNA
gaaccatggttcagcatcagacaatttgc

SEQ ID NO: 251 - DNA, 34 bp, synthetic construct
  misc_feature 1..34: note = tcdC Reverse Primer
  source 1..34: mol_type = other DNA
ctttcttttcgtcgtctttcattttgaaccatgg

SEQ ID NO: 252 - DNA, 36 bp, synthetic construct
  misc_feature 1..36: note = tcdC Reverse Primer
  source 1..36: mol_type = other DNA
caatagctttcttttcgtcgtctttcattttgaacc

SEQ ID NO: 253 - DNA, 25 bp, synthetic construct
  misc_feature 1..25: note = tcdC Reverse Primer
  source 1..25: mol_type = other DNA
tcataagtaataccagtatcatatc

SEQ ID NO: 254 - DNA, 26 bp, synthetic construct
  misc_feature 1..26: note = tcdC Reverse Primer
  source 1..26: mol_type = other DNA
tcataagtaataccagtatcatatcc

SEQ ID NO: 255 - DNA, 30 bp, synthetic construct
  misc_feature 1..30: note = tcdC Reverse Primer
  source 1..30: mol_type = other DNA
gtcgtctttcattttgaaccatggttcagc

SEQ ID NO: 256 - DNA, 42 bp, synthetic construct
  misc_feature 1..42: note = tcdC Reverse Primer
  source 1..42: mol_type = other DNA
gtttctatttctgttttttgagaatctataactgatgtttga

SEQ ID NO: 257 - DNA, 24 bp, synthetic construct
  misc_feature 1..24: note = tcdC Reverse Primer
  source 1..24: mol_type = other DNA
cctcctcatggtcttcagaacaag

SEQ ID NO: 258 - DNA, 25 bp, synthetic construct
  misc_feature 1..25: note = tcdC Reverse Primer
  source 1..25: mol_type = other DNA
cctcctcatggtcttcagaacaagc

SEQ ID NO: 259 - DNA, 26 bp, synthetic construct
  misc_feature 1..26: note = tcdC Reverse Primer
  source 1..26: mol_type = other DNA
gacctcctcatggtcttcagaacaag

SEQ ID NO: 260 - DNA, 29 bp, synthetic construct
  misc_feature 1..29: note = tcdB Forward iPrimer
  source 1..29: mol_type = other DNA
gtaagtttaggtgcagcaatcaaagagct

SEQ ID NO: 261 - DNA, 29 bp, synthetic construct
  misc_feature 1..29: note = tcdB Forward iPrimer
  source 1..29: mol_type = other DNA
gtaagtttaggtgcagcaatcaaagagtt

SEQ ID NO: 262 - DNA, 39 bp, synthetic construct
  misc_feature 1..39: note = tcdB Forward iPrimer
  source 1..39: mol_type = other DNA
ggattacctgtaattgctactattatagatggtgtaagt

SEQ ID NO: 263 - DNA, 38 bp, synthetic construct
  misc_feature 1..38: note = tcdB Forward iPrimer
  source 1..38: mol_type = other DNA
gattacctgtaattgctactattatagatggtgtaagt

SEQ ID NO: 264 - DNA, 29 bp, synthetic construct
  misc_feature 1..29: note = tcdB Forward Primer
  source 1..29: mol_type = other DNA
gtgtaagtttaggtgcagcaatcaaagag

SEQ ID NO: 265 - DNA, 18 bp, synthetic construct
  misc_feature 1..18: note = tcdB Forward Primer
  source 1..18: mol_type = other DNA
ggtgcagcaatcaaagag

SEQ ID NO: 266 - DNA, 31 bp, synthetic construct
  misc_feature 1..31: note = tcdB Forward Primer
  source 1..31: mol_type = other DNA
tggtgtaagtttaggtgcagcaatcaaagag

SEQ ID NO: 267 - DNA, 34 bp, synthetic construct
  misc_feature 1..34: note = tcdB Forward Primer
  source 1..34: mol_type = other DNA
gattggaggtcaaataaatgacactgctattaat

SEQ ID NO: 268 - DNA, 30 bp, synthetic construct
  misc_feature 1..30: note = tcdB Forward Primer
  source 1..30: mol_type = other DNA
atagatggtgtaagtttaggtgcagcaatc

SEQ ID NO: 269 - DNA, 31 bp, synthetic construct
  misc_feature 1..31: note = tcdB Forward Primer
  source 1..31: mol_type = other DNA
tggtgtaagtttaggtgcatcaattaaagag

SEQ ID NO: 270 - DNA, 25 bp, synthetic construct
  misc_feature 1..25: note = tcdB Forward Primer
  source 1..25: mol_type = other DNA
ggtgcagcaatcaaagagctaagtg

SEQ ID NO: 271 - DNA, 34 bp, synthetic construct
  misc_feature 1..34: note = tcdB Forward Primer
  source 1..34: mol_type = other DNA
gtaagtttaggtgcatcaattaaagagttgagtg

SEQ ID NO: 272 - DNA, 36 bp, synthetic construct
  misc_feature 1..36: note = tcdB Forward Primer
  source 1..36: mol_type = other DNA
gtgtaagtttaggtgcatcaattaaagagttgagtg

SEQ ID NO: 273 - DNA, 24 bp, synthetic construct
  misc_feature 1..24: note = tcdB Forward Primer
  source 1..24: mol_type = other DNA
gtggatttagtatacttttagttc

SEQ ID NO: 274 - DNA, 35 bp, synthetic construct
  misc_feature 1..35: note = tcdB Forward Primer
  source 1..35: mol_type = other DNA
gtattacctaatgctccaaatagagtatttgcttg

SEQ ID NO: 275 - DNA, 29 bp, synthetic construct
  misc_feature 1..29: note = tcdB Forward Primer
  source 1..29: mol_type = other DNA
cctaatgccccagatagagtatttggctg

SEQ ID NO: 276 - DNA, 23 bp, synthetic construct
  misc_feature 1..23: note = tcdB Forward Primer
  source 1..23: mol_type = other DNA
gccccagatagaatctttggctg

SEQ ID NO: 277 - DNA, 28 bp, synthetic construct
  misc_feature 1..28: note = tcdB Forward Primer
  source 1..28: mol_type = other DNA
ctaatgccccagatagaatctttggctg

SEQ ID NO: 278 - DNA, 24 bp, synthetic construct
  misc_feature 1..24: note = tcdB Forward Primer
  source 1..24: mol_type = other DNA
cttgatttgtcaaaagatttaatg

SEQ ID NO: 279 - DNA, 24 bp, synthetic construct
  misc_feature 1..24: note = tcdB Forward Primer
  modified_base 1: mod_base = OTHER; note = n is 5-methylated cytosine
  modified_base 11: mod_base = OTHER; note = n is 5-methylated cytosine
  source 1..24: mol_type = other DNA
nttgatttatnaaaagatttaatg

SEQ ID NO: 280 - DNA, 35 bp, synthetic construct
  misc_feature 1..35: note = tcdB Invader
  source 1..35: mol_type = other DNA
tttactgccattatacctatcttagcttctattta

SEQ ID NO: 281 - DNA, 22 bp, synthetic construct
  misc_feature 1..22: note = THS for tcdB Primary Probe
  source 1..22: mol_type = other DNA
cttgtcttaataatgggtcact

SEQ ID NO: 282 - DNA, 35 bp, synthetic construct
  misc_feature 1..35: note = THS for tcdB Primary Probe
  source 1..35: mol_type = other DNA
taattatataaatcaatggaaagatgtaaatagtg

SEQ ID NO: 283 - DNA, 17 bp, synthetic construct
  misc_feature 1..17: note = THS for tcdB Primary Probe
  source 1..17: mol_type = other DNA
gggaaagaggatggacg

SEQ ID NO: 284 - DNA, 18 bp, synthetic construct
  misc_feature 1..18: note = THS for tcdB Primary Probe
  source 1..18: mol_type = other DNA
gggaaagaggatggacgc

SEQ ID NO: 285 - DNA, 16 bp, synthetic construct
  misc_feature 1..16: note = THS for tcdB Primary Probe
  source 1..16: mol_type = other DNA
gggaaagaggatggac

SEQ ID NO: 286 - DNA, 21 bp, synthetic construct
  misc_feature 1..21: note = THS for tcdB Primary Probe
  source 1..21: mol_type = other DNA
gggaaagaggatggacgccag

SEQ ID NO: 287 - DNA, 19 bp, synthetic construct
  misc_feature 1..19: note = THS for tcdB Primary Probe
  source 1..19: mol_type = other DNA
gggaaacaggatggacacc

SEQ ID NO: 288 - DNA, 19 bp, synthetic construct
  misc_feature 1..19: note = THS for tcdB Primary Probe
  source 1..19: mol_type = other DNA
gggaaagaggatggacgcc

SEQ ID NO: 289 - DNA, 38 bp, synthetic construct
  misc_feature 1..38: note = THS for tcdB Primary Probe
  source 1..38: mol_type = other DNA
attatagtcactatttacatctttccattgatttatat

SEQ ID NO: 290 - DNA, 25 bp, synthetic construct
  misc_feature 1..25: note = THS for tcdB Primary Probe
  source 1..25: mol_type = other DNA
caaagagctaagtgaaaccagtgac

SEQ ID NO: 291 - DNA, 26 bp, synthetic construct
  misc_feature 1..26: note = THS for tcdB Primary Probe
  source 1..26: mol_type = other DNA
caaagagctaagtgaaaccagtgacc

SEQ ID NO: 292 - DNA, 27 bp, synthetic construct
  misc_feature 1..27: note = THS for tcdB Primary Probe
  source 1..27: mol_type = other DNA
caaagagctaagtgaaaccagtgaccc

SEQ ID NO: 293 - DNA, 30 bp, synthetic construct
  misc_feature 1..30: note = THS for tcdB Primary Probe
  source 1..30: mol_type = other DNA
taagtgaaaccagtgacccattattaagac

SEQ ID NO: 294 - DNA, 22 bp, synthetic construct
  misc_feature 1..22: note = THS for tcdB Primary Probe
  source 1..22: mol_type = other DNA
cttgtcttaataatgggtcact

SEQ ID NO: 295 - DNA, 30 bp, synthetic construct
  misc_feature 1..30: note = THS for tcdB Primary Probe
  source 1..30: mol_type = other DNA
tgagtgaaaccagtgacccattattaagac

SEQ ID NO: 296 - DNA, 25 bp, synthetic construct
  misc_feature 1..25: note = THS for tcdB Primary Probe
  source 1..25: mol_type = other DNA
tttaggtgcatcaattaaagagttg

SEQ ID NO: 297 - DNA, 25 bp, synthetic construct
  misc_feature 1..25: note = THS for tcdB Primary Probe
  source 1..25: mol_type = other DNA
tttaggtgcagcaattaaagagtta

SEQ ID NO: 298 - DNA, 22 bp, synthetic construct
  misc_feature 1..22: note = THS for tcdB Primary Probe
  modified_base 6: mod_base = OTHER; note = n is 5-methylated cytosine
  modified_base 19: mod_base = OTHER; note = n is 5-methylated cytosine
  modified_base 21: mod_base = OTHER; note = n is 5-methylated cytosine
  source 1..22: mol_type = other DNA
cttgtnttaataatgggtnant

SEQ ID NO: 299 - DNA, 32 bp, synthetic construct
  misc_feature 1..32: note = tcdB Primary Probe
  misc_feature 1..10: note = RNA
  misc_feature 11..32: note = DNA
  modified_base 32: mod_base = OTHER; note = 3'-terminal Hexanediol
  source 1..32: mol_type = other DNA
cgcgccgaggcttgtcttaataatgggtcact

SEQ ID NO: 300 - DNA, 47 bp, synthetic construct
  misc_feature 1..47: note = tcdB Primary Probe
  misc_feature 1..12: note = RNA
  misc_feature 13..47: note = DNA
  modified_base 47: mod_base = OTHER; note = 3'-terminal Hexanediol
  source 1..47: mol_type = other DNA
acggacgcggagtaattatataaatcaatggaaagatgtaaatagtg

SEQ ID NO: 301 - DNA, 29 bp, synthetic construct
  misc_feature 1..29: note = tcdB Primary Probe
  misc_feature 1..12: note = RNA
  misc_feature 13..29: note = DNA
  modified_base 29: mod_base = OTHER; note = 3'-terminal Hexanediol
  source 1..29: mol_type = other DNA
acggacgcggaggggaaagaggatggacg

SEQ ID NO: 302 - DNA, 30 bp, synthetic construct
  misc_feature 1..30: note = tcdB Primary Probe
  misc_feature 1..12: note = RNA
  misc_feature 13..30: note = DNA
  modified_base 30: mod_base = OTHER; note = 3'-terminal Hexanediol
  source 1..30: mol_type = other DNA
acggacgcggaggggaaagaggatggacgc

SEQ ID NO: 303 - DNA, 28 bp, synthetic construct
  misc_feature 1..28: note = tcdB Primary Probe
  misc_feature 1..12: note = RNA
  misc_feature 13..28: note = DNA
  modified_base 28: mod_base = OTHER; note = 3'-terminal Hexanediol
  source 1..28: mol_type = other DNA
acggacgcggaggggaaagaggatggac

SEQ ID NO: 304 - DNA, 33 bp, synthetic construct
  misc_feature 1..33: note = tcdB Primary Probe
  misc_feature 1..12: note = RNA
  misc_feature 13..33: note = DNA
  modified_base 33: mod_base = OTHER; note = 3'-terminal Hexanediol
  source 1..33: mol_type = other DNA
acggacgcggaggggaaagaggatggacgccag

SEQ ID NO: 305 - DNA, 31 bp, synthetic construct
  misc_feature 1..31: note = tcdB Primary Probe
  misc_feature 1..12: note = RNA
  misc_feature 13..31: note = DNA
  modified_base 31: mod_base = OTHER; note = 3'-terminal Hexanediol
  source 1..31: mol_type = other DNA
acggacgcggaggggaaacaggatggacacc

SEQ ID NO: 306 - DNA, 31 bp, synthetic construct
  misc_feature 1..31: note = tcdB Primary Probe
  misc_feature 1..12: note = RNA
  misc_feature 13..31: note = DNA
  modified_base 31: mod_base = OTHER; note = 3'-terminal Hexanediol
  source 1..31: mol_type = other DNA
acggacgcggaggggaaagaggatggacgcc

SEQ ID NO: 307 - DNA, 50 bp, synthetic construct
  misc_feature 1..50: note = tcdB Primary Probe
  misc_feature 1..12: note = RNA
  misc_feature 13..50: note = DNA
  modified_base 50: mod_base = OTHER; note = 3'-terminal Hexanediol
  source 1..50: mol_type = other DNA
acggacgcggagattatagtcactatttacatctttccattgatttatat

SEQ ID NO: 308 - DNA, 37 bp, synthetic construct
  misc_feature 1..37: note = tcdB Primary Probe
  modified_base 37: mod_base = OTHER; note = 3'-terminal Hexanediol
  source 1..37: mol_type = other DNA
acggacgcggagcaaagagctaagtgaaaccagtgac

SEQ ID NO: 309 - DNA, 38 bp, synthetic construct
  misc_feature 1..38: note = tcdB Primary Probe
  modified_base 38: mod_base = OTHER; note = 3'-terminal Hexanediol
  source 1..38: mol_type = other DNA
acggacgcggagcaaagagctaagtgaaaccagtgacc

SEQ ID NO: 310 - DNA, 39 bp, synthetic construct
  misc_feature 1..39: note = tcdB Primary Probe
  modified_base 39: mod_base = OTHER; note = 3'-terminal Hexanediol
  source 1..39: mol_type = other DNA
acggacgcggagcaaagagctaagtgaaaccagtgaccc

SEQ ID NO: 311 - DNA, 42 bp, synthetic construct
  misc_feature 1..42: note = tcdB Primary Probe
  modified_base 42: mod_base = OTHER; note = 3'-terminal Hexanediol
  source 1..42: mol_type = other DNA
acggacgcggagtaagtgaaaccagtgacccattattaagac

SEQ ID NO: 312 - DNA, 34 bp, synthetic construct
  misc_feature 1..34: note = tcdB Primary Probe
  misc_feature 1..12: note = RNA
  misc_feature 13..34: note = DNA
  modified_base 34: mod_base = OTHER; note = 3'-terminal Hexanediol
  source 1..34: mol_type = other DNA
acggacgcggagcttgtcttaataatgggtcact

SEQ ID NO: 313 - DNA, 42 bp, synthetic construct
  misc_feature 1..42: note = tcdB Primary Probe
  modified_base 42: mod_base = OTHER; note = 3'-terminal Hexanediol
  source 1..42: mol_type = other DNA
acggacgcggagtgagtgaaaccagtgacccattattaagac

SEQ ID NO: 314 - DNA, 37 bp, synthetic construct
  misc_feature 1..37: note = tcdB Primary Probe
  misc_feature 1..12: note = RNA
  misc_feature 13..37: note = DNA
  modified_base 37: mod_base = OTHER; note = 3'-terminal Hexanediol
  source 1..37: mol_type = other DNA
acggacgcggagtttaggtgcatcaattaaagagttg

SEQ ID NO: 315 - DNA, 37 bp, synthetic construct
  misc_feature 1..37: note = tcdB Primary Probe
  misc_feature 1..12: note = RNA
  misc_feature 13..37: note = DNA
  modified_base 37: mod_base = OTHER; note = 3'-terminal Hexanediol
  source 1..37: mol_type = other DNA
acggacgcggagtttaggtgcagcaattaaagagtta

SEQ ID NO: 316 - DNA, 34 bp, synthetic construct
  misc_feature 1..34: note = tcdB Primary Probe
  misc_feature 1..12: note = RNA
  misc_feature 13..34: note = DNA
  modified_base 18: mod_base = OTHER; note = n is 5-methylated cytosine
  modified_base 31: mod_base = OTHER; note = n is 5-methylated cytosine
  modified_base 33: mod_base = OTHER; note = n is 5-methylated cytosine
  modified_base 34: mod_base = OTHER; note = 3'-terminal Hexanediol
  source 1..34: mol_type = other DNA
acggacgcggagcttgtnttaataatgggtnant

SEQ ID NO: 317 - DNA, 35 bp, synthetic construct
  misc_feature 1..35: note = tcdB Reverse iPrimer
  source 1..35: mol_type = other DNA
tttactgccattatacctatcttagcttctatttc

SEQ ID NO: 318 - DNA, 36 bp, synthetic construct
  misc_feature 1..36: note = tcdB Reverse Primer
  source 1..36: mol_type = other DNA
ctgccattatacctatcttagcttctatttcttgtc

SEQ ID NO: 319 - DNA, 34 bp, synthetic construct
  misc_feature 1..34: note = tcdB Reverse Primer
  source 1..34: mol_type = other DNA
ctgccattatacctatcttagcttctatttcttg

SEQ ID NO: 320 - DNA, 34 bp, synthetic construct
  misc_feature 1..34: note = tcdB Reverse Primer
  source 1..34: mol_type = other DNA
ctgccattatacctatttttgcttctatttcttg

SEQ ID NO: 321 - DNA, 29 bp, synthetic construct
  misc_feature 1..29: note = tcdB Forward Primer
  source 1..29: mol_type = other DNA
gtaagtttaggtgcatcaattaaagagtt

SEQ ID NO: 322 - DNA, 28 bp, synthetic construct
  misc_feature 1..28: note = tcdB Reverse Primer
  source 1..28: mol_type = other DNA
gaactaaaagtatactaaatccactagc

SEQ ID NO: 323 - DNA, 47 bp, synthetic construct
  misc_feature 1..47: note = tcdB Reverse iPrimer
  source 1..47: mol_type = other DNA
tcaatgtgtttatcaaaaatgcattactatcataaaaaacattaaca

SEQ ID NO: 324 - DNA, 35 bp, synthetic construct
  misc_feature 1..35: note = tcdB Reverse iPrimer
  source 1..35: mol_type = other DNA
tttactgccattatacctatttttgcttctatttc

SEQ ID NO: 325 - DNA, 35 bp, synthetic construct
  misc_feature 1..35: note = tcdB Reverse iPrimer
  source 1..35: mol_type = other DNA
tttactgccattatacctattttggcttctatttc

SEQ ID NO: 326 - DNA, 36 bp, synthetic construct
  misc_feature 1..36: note = tcdB Reverse iPrimer
  source 1..36: mol_type = other DNA
gtttactgccattatacctattttggcttctatttc

SEQ ID NO: 327 - DNA, 47 bp, synthetic construct
  misc_feature 1..47: note = tcdB Reverse Primer
  source 1..47: mol_type = other DNA
caatgtgtttatcaaaaatgcattactatcataaaaaacattaacat

SEQ ID NO: 328 - DNA, 36 bp, synthetic construct
  misc_feature 1..36: note = tcdB Reverse Primer
  source 1..36: mol_type = other DNA
ctatttcttgtcttaataatgggtcacttgtttcac

SEQ ID NO: 329 - DNA, 30 bp, synthetic construct
  misc_feature 1..30: note = tcdB Reverse Primer
  source 1..30: mol_type = other DNA
cttgtcttaataatgggtcacttgtttcac

SEQ ID NO: 330 - DNA, 36 bp, synthetic construct
  misc_feature 1..36: note = tcdB Reverse Primer
  source 1..36: mol_type = other DNA
ctatttcttgtcttaataatgggtcactagtttcac

SEQ ID NO: 331 - DNA, 30 bp, synthetic construct
  misc_feature 1..30: note = tcdB Reverse Primer
  source 1..30: mol_type = other DNA
cttgtcttaataatgggtcactagtttcac

SEQ ID NO: 332 - DNA, 44 bp, synthetic construct
  misc_feature 1..44: note = tcdB Reverse Primer
  source 1..44: mol_type = other DNA
cctgctgaaattcctgctaaaggaactaaaagtatactaaatcc

SEQ ID NO: 333 - DNA, 43 bp, synthetic construct
  misc_feature 1..43: note = tcdB Reverse Primer
  source 1..43: mol_type = other DNA
cctgctgaaattcctgctaaaggaactaaaagtatactaaatc

SEQ ID NO: 334 - DNA, 38 bp, synthetic construct
  misc_feature 1..38: note = tcdB Reverse Primer
  source 1..38: mol_type = other DNA
acctgctgaaattcctgctaaaggaactaaaagtatac

SEQ ID NO: 335 - DNA, 35 bp, synthetic construct
  misc_feature 1..35: note = tcdB Reverse Primer
  source 1..35: mol_type = other DNA
agttttgtaccatcattttctaagcttcttaaacc

SEQ ID NO: 336 - DNA, 36 bp, synthetic construct
  misc_feature 1..36: note = tcdB Reverse Primer
  source 1..36: mol_type = other DNA
cagttttgtgccatcattttctaagcttcttaaacc

SEQ ID NO: 337 - DNA, 19 bp, synthetic construct
  misc_feature 1..19: note = tcdB Reverse Primer
  source 1..19: mol_type = other DNA
ctcttatacggtctaacag

SEQ ID NO: 338 - DNA, 19 bp, synthetic construct
  misc_feature 1..19: note = tcdB Reverse Primer
  modified_base 1: mod_base = OTHER; note = n is 5-methylated cytosine
  modified_base 9: mod_base = OTHER; note = n is 5-methylated cytosine
  source 1..19: mol_type = other DNA
ntcttatanggtctaatag

SEQ ID NO: 339 - DNA, 33 bp, synthetic construct
  misc_feature 1..33: note = tcdB Forward Primer
  source 1..33: mol_type = other DNA
tggattggaggtcaaataaatgatactgctatt